SEQUENCE LISTING (10 sequences)

SEQ ID NO: 1 - protein, 868 aa, Rattus norvegicus
MATACKRSSGESQSEDIEASRMKRAAAKHLIERYYHQLTEGCGNEACTNEFCASCPTFLRMDNNAAAIKALELYKINAKLCDPHPSKKGASSAYLENSKGASNNSEIKMNKKEEFKEVDYLTEEKVYEIYEFCREREDYSPLIRVIGRIFSSAEALVQSFRKVKQHTKEELKSLQEKDEDKDEDEKEKAACSAAAMEEDSEASSSRMGDSSQGDNNVQKLGPDDVTVDIDAIRRVYSSLLSDEKIETAFLNALVYLSPNVECDLTYHNVYTRDPNYLNLFIIVMENRNLHSPEYLEMALPLFCKAMSKLPLAAQGKLIRLWSKYSADQIRRMMETFQQLITYKVISNEFNSRNLVNDDDAIVAASKCLKMVYYANVVGGDVDTNHNEEDDEEPIPESSELTLQELLGEERRNKKGPRVDPLETEIGVKTLDCRKPLISFEEFINEPLNDVLEMDKDYTFFKVETENKFSFMTCPFILNAVTKNLGLYYDNRIRMYSERRITVLYSLVQGQQLNPYLRLKVRRDHIIDDALVRLEMIAMENPADLKKQLYVEFEGEQGVDEGGVSKEFFQLVVEEIFNPDIGMFTYDEATRLFWFNPSSFETEGQFTLIGIVLGLAIYNNCILDVHFPMVVYRKLMGKKGTFCDLGDSHPILYQSLKDLLEYEGNVEDDMMITFQISQTDLFGNPMMYDLKENGDKIPITNENRKEFVSLYSDYILNKSVEKQFKAFRRGFHMVTNESPLKYLFRPEEIELLICGSRNLDFQALEETTEYDGGYTRESVVIREFWEIVHSFTDEQKRLFLQFTTGTDRAPVGGLGKLKMIIAKNGPDTERLPTSHTCFNVLLLPEYSSKEKLKERLLKAITYAKGFGML

SEQ ID NO: 2 - DNA, 2767 bp, Homo sapiens
  source 1..2767: mol_type = other DNA
acagtatgacatctgatgctggagggtcgcactttcacaaatgagtcagctggtacatggggttatcatcaatttttagctcttctgtctgggagatacaagtttggaagcaatcttggggtacttacccacaaggctggtggagaccagatcaggagaacctcagtctgacgacattgaagctagccgaatgaagcgagcagctgcaaagcatctaatagaacgctactaccaccagttaactgagggctgtggaaatgaagcctgcacgaatgagttttgtgcttcctgtccaacttttcttcgtatggataataatgcagcagctattaaagccctcgagctttataagattaatgcaaaactctgtgatcctcatccctccaagaaaggagcaagctcagcttaccttgagaactcgaaaggtgcccccaacaactcctgctctgagataaaaatgaacaagaaaggcgctagaattgattttaaagatgtgacttacttaacagaagagaaggtatatgaaattcttgaattatgtagagaaagagaggattattcccctttaatccgtgttattggaagagttttttctagtgctgaggcattggtacagagcttccggaaagttaaacaacacaccaaggaagaactgaaatctcttcaagcaaaagatgaagacaaagatgaggatgaaaaggaaaaagctgcatgttctgctgctgctatggaagaagactcagaagcatcttcctcaaggataggtgatagctcacagggagacaacaatttgcaaaaattaggccctgatgatgtgtctgtggatattgatgccattagaagggtctacaccagattgctctctaatgaaaaaattgaaactgcctttctcaatgcacttgtatatttgtcacctaacgtggaatgtgacttgacgtatcacaatgtatactctcgagatcctaattatctgaatttgttcattatcgtaatggagaatagaaatctccacagtcctgaatatctggaaatggctttgccattattttgcaaagcgatgagcaagctaccccttgcagcccaaggaaaactgatcagactgtggtctaaatacaatgcagaccagattcggagaatgatggagacatttcagcaacttattacttataaagtcataagcaatgaatttaacagtcgaaatctagtgaatgatgatgatgccattgttgctgcttcgaagtgcttgaaaatggtttactatgcaaatgtagtgggaggggaagtggacacaaatcacaatgaagaagatgatgaagagcccatccctgagtccagcgagctgacacttcaggaacttttgggagaagaaagaagaaacaagaaaggtcctcgagtggaccccctggaaactgaacttggtgttaaaaccctggattgtcgaaaaccacttatcccttttgaagagtttattaatgaaccactgaatgaggttctagaaatggataaagattatacttttttcaaagtagaaacagagaacaaattctcttttatgacatgtccctttatattgaatgctgtcacaaagaatttgggattatattatgacaatagaattcgcatgtacagtgaacgaagaatcactgttctctacagcttagttcaaggacagcagttgaatccatatttgagactcaaagttagacgtgaccatatcatagatgatgcacttgtccggctagagatgatcgctatggaaaatcctgcagacttgaagaagcagttgtatgtggaatttgaaggagaacaaggagttgatgagggaggtgtttccaaagaattttttcagctggttgtggaggaaatcttcaatccagatattggtatgttcacatacgatgaatctacaaaattgttttggtttaatccatcttcttttgaaactgagggtcagtttactctgattggcatagtactgggtctggctatttacaataactgtatactggatgtacattttcccatggttgtctacaggaagctaatggggaaaaaaggaacttttcgtgacttgggagactctcacccagttctatatcagagtttaaaagatttattggagtatgaagggaatgtggaagatgacatgatgatcactttccagatatcacagacagatctttttggtaacccaatgatgtatgatctaaaggaaaatggtgataaaattccaattacaaatgaaaacaggaaggaatttgtcaatctttattctgactacattctcaataaatcagtagaaaaacagttcaaggcttttcggagaggttttcatatggtgaccaatgaatctcccttaaagtacttattcagaccagaagaaattgaattgcttatatgtggaagccggaatctagatttccaagcactagaagaaactacagaatatgacggtggctataccagggactctgttctgattagggagttctgggaaatcgttcattcatttacagatgaacagaaaagactcttcttgcagtttacaacgggcacagacagagcacctgtgggaggactaggaaaattaaagatgattatagccaaaaatggcccagacacagaaaggttacctacatctcatacttgctttaatgtgcttttacttccggaatactcaagcaaagaaaaacttaaagagagattgttgaaggccatcacgtatgccaaaggatttggcatgctgtaaaacaaaacaaaacaaaat

SEQ ID NO: 3 - DNA, 57 bp, Homo sapiens
  source 1..57: mol_type = other DNA
atgaagttatgggatgtcgtggctgtctgcctggtgctgctccacaccgcgtccgcc

SEQ ID NO: 4 - DNA, 33 bp, Human immunodeficiency virus 1
  source 1..33: mol_type = other DNA
tacggcagaaagaagaggaggcagagaaggaga

SEQ ID NO: 5 - DNA, 72 bp, Homo sapiens
  source 1..72: mol_type = other DNA
atggccctgtggatgcgcctcctgcccctgctggcgctgctggccctctggggacctgacccagccgcagcc

SEQ ID NO: 6 - DNA, 60 bp, Homo sapiens
  source 1..60: mol_type = other DNA
atggagacagacacactcctgctatgggtactgctgctctgggttccaggttccactggt

SEQ ID NO: 7 - DNA, 10893 bp, Homo sapiens
  misc_difference 122: note = n is a, c, g or t
  misc_difference 340: note = n is a, c, g, or t
  misc_difference 1145: note = n is a, c, g, or t
  misc_difference 1180: note = n is a, c, g, or t
  misc_difference 1257: note = n is a, c, g, or t
  misc_difference 5410: note = n is a, c, g, or t
  misc_difference 7624: note = n is a, c, g, or t
  misc_difference 7644: note = n is a, c, g, or t
  misc_difference 7647: note = n is a, c, g, or t
  misc_difference 7741: note = n is a, c, g, or t
  misc_difference 7836: note = n is a, c, g, or t
  source 1..10893: mol_type = other DNA
ggagtagtttactgagccactaatctaaagtttaatactgtgagtgaataccagtgagtacctttgttaatgtggataaccaatacttggctataggaagttttttagttgtgtgttttatnacacgtatttgactttgtgaataattatggcttataatggcttgtctgttggtatctatgtatagcgtttacagtttcctttaaaaaacatgcattgagttttttaatagtccaacccttaaaataaatgtgttgtatggccacctgatctgaccactttctttcatgttgacatctttaattttaaaactgttttatttagtgcttaaatcttgttnacaaaattgtcttcctaagtaatatgtctacctttttttttggaatatggaatattttgctaactgtttctcaattgcattttacagatcaggagaacctcagtctgacgacattgaagctagccgaatgtaagtgtaacttggttgagactgtggttcttattttgagttgccctagactgctttaaattacgtcacattatttggaaataatttctggttaaaagaaaggaatcatttagcagtaaatgggagataggaacatacctactttttttcctatcagataactctaaacctcggtaacagtttactaggtttctactactagatagataaatgcacacgcctaaattcttagtctttttgcttccctggtagcagttgtagggaaatagggaggttgaggaaagagtttaacagtctcaacgcctaccatatttaaggcatcaagtactatgttatagatacagagatgcgtaataattagttttcaccctacagaaatttatattatactcaagagtgaaagatgcagaagcaaataatttcagtcactgaggtagaatggtatccaaaatacaatagtaacatgaaggagtactggagtaccaggtatgcaataggaatctagtgtagatggcagggaagtaagagtggccaggaaatgctaagttcagtcttgaaatgtgactgggaatcaggcagctatcaactataagtcaaatgtttacaagctgttaaaaatgaaatactgattatgtaaaagaaaaccggattgatgctttaaatagactcattttcntaatgctaatttttaaaatgatagaatcctacaantcttagctgtaaaccttgtgatttttcagctgttgtactaaacaacttaagcacatataccatcagacaagcccccntccccccttttaaaccaaaggaatgtatactctgttaatacagtcagtaagcattgacattctttatcataatatcctagaaaatatttattaactatttcactagtcaggagttgtggtaaatagtgcatctccattttctacttctcatcttcatacacaggttaatcacttcagtgcttgactaacttttgccttgatgatatgttgagctttgtacttgagagctgtactaatcactgtgcttattgtttgaatgtttggtacaggaagcgagcagctgcaaagcatctaatagaacgctactaccaccagttaactgagggctgtggaaatgaagcctgcacgaatgagttttgtgcttcctgtccaacttttcttcgtatggataataatgcagcagctattaaagccctcgagctttataagattaatgcaaaactctgtgatcctcatccctccaagaaaggagcaagctcagcttaccttgagaactcgaaaggtgcccccaacaactcctgctctgagataaaaatgaacaagaaaggcgctagaattgattttaaaggtaagatgttttattttcaattgagaattgttgcctgaaaaccatgtgggagatttaaatgtattagtttttatttgttttttcttctgtgacataaagacattttgatatcgtagaaccaattttttattgtggtaacggacaggaataataactacattttacaggtctaatcattgctaattagaagcagatcatatgccaaaagttcatttgttaatagattgatttgaactttttaaaattcttaggaaaaatgtattaagtggtagtgaatctccaaaactatttaagagctgtattatgattaatcagtacatgacatattggttcatatttataattaaagctatacattaatagatatcttgattataaagaaagtttaaactcatgatcttattaagagttatacattgttgaaagaatgtaaaagcatgggtgaggtcattggtataggtaggtagttcattgaaaaaaataggtaagcattaaattttgtttgctgaatctaagtattagatactttaagagttgtatatcataaatgatattgagcctagaatgtttggctgttttacttttagaactttttgcaacagagtaaacatacatattatgaaaataaatgttctcttttttcctctgattttctagatgtgacttacttaacagaagagaaggtatatgaaattcttgaattatgtagagaaagagaggattattcccctttaatccgtgttattggaagagttttttctagtgctgaggcattggtacagagcttccggaaagttaaacaacacaccaaggaagaactgaaatctcttcaagcaaaagatgaagacaaagatgaagatgaaaaggaaaaagctgcatgttctgctgctgctatggaagaagactcagaagcatcttcctcaaggataggtgatagctcacagggagacaacaatttgcaaaaattaggccctgatgatgtgtctgtggatattgatgccattagaagggtctacaccagattgctctctaatgaaaaaattgaaactgcctttctcaatgcacttgtatatttgtcacctaacgtggaatgtgacttgacgtatcacaatgtatactctcgagatcctaattatctgaatttgttcattatcgtaatggagaatagaaatctccacagtcctgaatatctggaaatggctttgccattattttgcaaagcgatgagcaagctaccccttgcagcccaaggaaaactgatcagactgtggtctaaatacaatgcagaccagattcggagaatgatggagacatttcagcaacttattacttataaagtcataagcaatgaatttaacagtcgaaatctagtgaatgatgatgatgccattgttgctgcttcgaagtgcttgaaaatggtttactatgcaaatgtagtgggaggggaagtggacacaaatcacaatgaagaagatgatgaagagcccatccctgagtccagcgagctgacacttcaggaacttttgggagaagaaagaagaaacaagaaaggtcctcgagtggaccccctggaaactgaacttggtgttaaaaccctggattgtcgaaaaccacttatcccttttgaagagtttattaatgaaccactgaatgaggttctagaaatggataaagattatacttttttcaaagtagaaacagagaacaaattctcttttatgacatgtccctttatattgaatgctgtcacaaagaatttgggattatattatgacaatagaattcgcatgtacagtgaacgaagaatcactgttctctacagcttagttcaaggacagcagttgaatccatatttgagactcaaagttagacgtgaccatatcatagatgatgcacttgtccgggtaagttgggctgctagattaaaaacctaataatggggatatcatgatacagttcagtgaattcattttaaaagtgactgaaaaaaatgataccatatagcataggaacacatggacatttctgatcttatataagtattatacttttgttgttcctgtgcaagtttatagatgtgttctacaaagtatcggttgtattatataatggtcatgctatctttgaaaaagaatgggttttctaaatcttgaaaactaaatccaaagtttctttcattcagaagagaatagagtgttggacaaagaccagaacaagagaaatgtggagatacccaataataagtgtggatgtgcagtcttgaactgggagtaatggtacagtaaaaccataccataaaattataggtagtgtccaaaaaattccatcgtgtaaaattcagagttgcattattgtggacttgaagaagcagttgtatgtgggacggtatcgataagcttgatatcgaattcctgcagcccgggggatccactagtgtggtaattaatactaagtcttactgtgagagaccataaactgctttagtattcagtgtatttttcttaattgaaatatttaacttatgacttagtagatactaagacttaacccttgagtttctattctaataaaggactactaatgaacaattttgaggttagacctctactccattgtttttgctgaaatgatttagctgcttttccatgtcctgtgtagtccagacttaacacacaagtaataaaatcttaattaattgtatgttaatttcataacaaatcagtaaagttagctttttactatgctagtgtctgttttgtgtctgtctttttgattatctttaagactgaatctttgtcttcactggctttttatcagtttgctttctgtttccatttacatacaaaaagtcaaaaatttgtatttgtttcctaatcctactccttgtttttattttgtttttttcctgatactagcaatcatcttcttttcatgtttatcttttcaatcactagctagagatgatcgctatggaaaatcctgcagacttgaagaagcagttgtatgtggaatttgaaggagaacaaggagttgatgagggaggtgtttccaaagaattttttcagctggttgtggaggaaatcttcaatccagatattggtaaatacattagtaatgtgattatggtgtcgtatcatcttttgagttagttatttgtttatcttactttgtaaatattttcagctatgaagagcagcaaaagaaggatttggtatggattacccagaatcacacatcatgactgaatttgtaggttttaggaactgatttgtatcactaatttattcaaattcttttatttcttagaaggaatattctaatgaaggaaattatctctttggtaaactgaattgaaagcactttagaatggtatattggaacagttggagggatttctttgctttttgttgtctaaaaccatcatcaaactcacggttttcctgacctgtgaacttcaaagaacaatggtttgaagagtattgagagactgtctcacaagtatgtcatgctcaaagttcagaaacactagctgatatcacattaattaggtttatttgctataagatttcttggggcttaatatangtagtgttcccccaaactttttgaactccagaactcttttctgccctaacagtagctactcaggagctgaggcaggagaattgtttgaacctaggaggcagaggttgcagtgagctgagatcgtgccactccagcccacccctgggtaacagagcgagactccatctcaaagaaaaaaatgaaaaattgttttcaaaaatagtacgtgtggtacagatataagtaattatatttttataaatgaaacactttggaaatgtagccattttttgtttttttatgtttatttttcagctatgggtggataaagcatgaatataacttttcttatgtgttagtagaaaattagaaagcttgaatttaattaacgtatttttctacccgatgccaccaaattacttactactttattcctttggcttcataaaattacatatcaccattcaccccaatttatagcagatatatgtggacattgttttctcaagtgctaatataatagaaatcaatgttgcatgcctaattacatatattttaaatgttttatatgcataattattttaagtttatatttgtattattcatcagtccttaataaaatacaaaagtaatgtatttttaaaaatcatttcttataggtatgttcacatacgatgaatctacaaaattgttttggtttaatccatcttcttttgaaactgagggtcagtttactctgattggcatagtactgggtctggctatttacaataactgtatactggatgtacattttcccatggttgtctacaggaagctaatggggaaaaaaggaacttttcgtgacttgggagactctcacccagtaagttctttgtcatttttttaattcagtctcttagattttatttaaatgcaaaaatttaatttatgtcaaaattttaaagtttttgtttagaatctttgttgatactcttatcaataagataaaaatgttttaatctgaccgaagtaccagaaacacttaaaaactcaaagggggacatttttatatattgctgtcagcacgaagctttcgtaagattgatttcatagagaagtgtttctaaacattttgtttgtgttttagtgaaatcttaagagataggtaaaaatcagagtagccctggctaagggtcttggtagttacaacgagtgtgcctgctcctaccacccccacccccaccttgagacaccacagaatttctcatagagcacagtgtgaattctattgctaaattggtggtatggggtttctcagcagagaatgggacatcacagtgactgacaatctttcttttataggttggaaactatttgggggactggagggatactgtctacactttttacaatttttattgataagatttttgttgtcttctaagaagagtgatataaattatttgttgtattttgtagttctatggtggcctcaatttaccatttctggttgctaggttctatatcagagtttaaaagatttattggagtatgaagggaatgtggaagatgacatgatgatcactttccagatatcacagacagatctttttggtaacccaatgatgtatgatctaaaggaaaatggtgataaaattccaattacaaatgaaaacaggaaggtaataaatgtttttatgtcacattttgtctcttcattaacactttcaaagcatgtatgcttataatttttaaagaagtatctaatatagtctgtacaaaaaaaaaacaagtaactaagtttatgtaaatgctagagtccacttttctaaatcttggatataagttggtatgaaagcacacagttgggcactaaagccccttttagagaaagaggacatgaagcaggagatagttaatagctaagtgtggttgtagtataaagcaagaagcagggtgtttcttgtattaagctgtaagcaggaacctcatgattaaggtctttatcacagaacaaataaaaattacatttaatttacacatgtatatcctgtttgtgataaaaatacatttctgaaaagtatactttacgtcagatttgggttctattgactaaaatgtgttcatcgggaatgggaataacccagaacataacaagcaaaaaattatgacaaatatatagtatacctttaagaaacatgtttatattgatataattttttgattaaatattatacacactaagggtacaangcacattttccttttatganttngatacagtagtttatgtgtcagtcagatacttccacatttttgctgaactggatacagtaagcagcttaccaaatattctatggtagaaaactnggacttcctggtttgcttaaatcaaatatattgtactctcttaaaacggttggcatttataaatagatggatacatggtttaaatgtgtctgttnacatacctagttgagagaacctaaagaattttctgcgtctccagcatttatattcagttctgtttaatacattatcgaaattgacatttataagtatgacagttttgtgtatatggccttttcatagcttaatattggctgtaacagagaattgtgaaattgtaagaagtagttttctttgtaggtgtaaaattgaatttttaagaatattcttgacagttttatgtatatggccttttcatagcttaatattggctataacagagaattgtgaaattgttaagaagtaggtgtaaaattgaatttttaagaatattcttgaatgtttttttcttggaaaaattaaaaagctatgcagcccaataacttgtgttttgtttgcatagcatattataagaagttcttgtgattaatgttttctacaggaatttgtcaatctttattctgactacattctcaataaatcagtagaaaaacagttcaaggcttttcggagaggttttcatatggtgaccaatgaatctcccttaaagtacttattcagaccagaagaaattgaattgcttatatgtggaagccgggtaagaaagcaggtgtctgcaaaaagtcatgtatcgatttattgtttgtaatgatacagtagtatagcagataactaagacatattttcttgaatttgcagaatctagatttccaagcactagaagaaactacagaatatgacggtggctataccagggactctgttctgattaggtgaggtacttagttcttcagaggaagatttgattcaccaaaggggtgtgtgattttgcttcagacctttatctctaggtactaattcccaaataagcaaactcacaaattgtcatctatatacttagatttgtatttgtaatataatcaccatttttcagagctaatcttgtgatttatttcatgaatgaagtgttgttatatataagtctcatgtaatctcctgcatttggcgtatggattatctagtattcctcactggttagagtatgcttactgctggttagaagataattaaaataaggctaccatgtctgcaatttttcctttcttttgaactctgcatttgtgaactgttacatggcttcccaggatcaagcactttttgagtgaaatggtagtcttttatttaattcttaagataatatgtccagatacatactagtatttccattttacaccctaaaaaactaagccctgaattctcacagaaagatgtagaggttcccagttctatctgcttttaaacaaatgcccttactactctactgtctacttctgtgtactacatcatcgtatgtagttgtttgcatttgggccagttggttggggcaggggtctttttttcttttgtcccttaatctgtatcactttttcctcccaaagttgagttaaaggatgagtagaccaggagaataaaggagaaaggataaataaaatatatacccaaaggcacctggagttaatttttccaaatattcatttcagtctttttcaattcataggattttgtcttttgctcattactgactgcataatgtgattataccatagtttaaatagtcacttcctgttactacacacttgggttttctcaattttttactattgtagtactaatattttactatattgtaatctaatccaaatttttacgtattcagagctgttcaggataaatttgcttggaaatttttaaatcaccagaagtgatactatcctgataattaacttccaagttgtctcttaatatagttttaatgcaaatcataagcttatgttagtaccagtcataatgaatgccaaactgaaaccagtattgtattttttctcattagggagttctgggaaatcgttcattcatttacagatgaacagaaaagactcttcttgcagtttacaacgggcacagacagagcacctgtgggaggactaggaaaattaaagatgattatagccaaaaatggcccagacacagaaaggtaggtaattattaacttgtgactgtatacctaccgaaaaccttgcattcctcgtcacatacatatgaactgtctttatagtttctgagcacattcgtgattttatatacaaatccccaaatcatattagacaattgagaaaatactttgctgtcattgtgtgaggaaacttttaagaaattgccctagttaaaaattattatggggctcacattggtttggaatcaaattagtgtgattcatttacttttttgattcccagcttgttaattgaaagccatataacatgatcatctatttagaatggttacattgaggctcggaagattatcatttgattgtgctagaatcctgttatcaaatcattttcttagtcatattgccagcagtgtttctaataagcatttaagagcacacactttgcagtcttgtaaaacaggtttgagtattttctccaccttagaggaagttacttgacttctcagtgacctaacctctaaagtgcatttactgatgtcctctctgtggttttgttgtggaaagatttagttaaatgaactgtaagaattcagtacctaaaatggtatctgttatgtagtaaaaactcaatggatacagtatcttatcatcgtcactagctttgagtaatttataggataaaggcaacttggtagttacacaacaaaaagtttatgatttgcattaatgtatagtttgcattgcagaccgtctcaactatatacaatctaaaaataggagcatttaattctaagtgtatttcccatgacttacagttttcctgtttttttccccttttctctatttaggttacctacatctcatacttgctttaatgtgcttttacttccggaatactcaagcaaagaaaaacttaaagagagattgttgaaggccatcacgtatgccaaaggatttggcatgctgtaaaacaaaacaaaacaaaataaaacaaaaaaaaggaaggaaaaaaaaagaaaaaatttaaaaaattttaaaaatataacgagggataaatttt

SEQ ID NO: 8 - protein, 852 aa, Homo sapiens
MKRAAAKHLIERYYHQLTEGCGNEACTNEFCASCPTFLRMDNNAAAIKALELYKINAKLCDPHPSKKGASSAYLENSKGAPNNSCSEIKMNKKGARIDFKDVTYLTEEKVYEILELCREREDYSPLIRVIGRVFSSAEALVQSFRKVKQHTKEELKSLQAKDEDKDEDEKEKAACSAAAMEEDSEASSSRIGDSSQGDNNLQKLGPDDVSVDIDAIRRVYTRLLSNEKIETAFLNALVYLSPNVECDLTYHNVYSRDPNYLNLFIIVMENRNLHSPEYLEMALPLFCKAMSKLPLAAQGKLIRLWSKYNADQIRRMMETFQQLITYKVISNEFNSRNLVNDDDAIVAASKCLKMVYYANVVGGEVDTNHNEEDDEEPIPESSELTLQELLGEERRNKKGPRVDPLETELGVKTLDCRKPLIPFEEFINEPLNEVLEMDKDYTFFKVETENKFSFMTCPFILNAVTKNLGLYYDNRIRMYSERRITVLYSLVQGQQLNPYLRLKVRRDHIIDDALVRLEMIAMENPADLKKQLYVEFEGEQGVDEGGVSKEFFQLVVEEIFNPDIGMFTYDESTKLFWFNPSSFETEGQFTLIGIVLGLAIYNNCILDVHFPMVVYRKLMGKKGTFRDLGDSHPVLYQSLKDLLEYEGNVEDDMMITFQISQTDLFGNPMMYDLKENGDKIPITNENRKEFVNLYSDYILNKSVEKQFKAFRRGFHMVTNESPLKYLFRPEEIELLICGSRNLDFQALEETTEYDGGYTRDSVLIREFWEIVHSFTDEQKRLFLQFTTGTDRAPVGGLGKLKMIIAKNGPDTERLPTSHTCFNVLLLPEYSSKEKLKERLLKAITYAKGFGML

SEQ ID NO: 9 - DNA, 5276 bp, Homo sapiens
  source 1..5276: mol_type = other DNA
agccagtcctcccgtcttgcgccgcggccgcgagatccgtgtgtctcccaagatggtggcgctgggctcggggtgactacaggagacgacggggccttttcccttcgccaggacccgacacaccaggcttcgctcgctcgcgcacccctccgccgcgtagccatccgccagcgcgggcgcccgccatccgccgcctacttacgcttcacctctgccgacccggcgcgctcggctgcgggcggcggcgcctccttcggctcctcctcggaatagctcgcggcctgtagcccctggcaggagggcccctcagccccccggtgtggacaggcagcggcggctggcgacgaacgccgggatttcggcggccccggcgctccctttcccggcctcgttttccggataaggaagcgcgggtcccgcatgagccccggcggtggcggcagcgaaagagaacgaggcggtggcgggcggaggcggcgggcgagggcgactacgaccagtgaggcggccgccgcagcccaggcgcgggggcgacgacaggttaaaaatctgtaagagcctgattttagaattcaccagctcctcagaagtttggcgaaatatgagttattaagcctacgctcagatcaaggtagcagctagactggtgtgacaacctgtttttaatcagtgactcaaagctgtgatcaccctgatgtcaccgaatggccacagcttgtaaaagagagttacagtggaggtaaaaggagtggcttgcaggatggagaagctgcaccagtgttattggaaatcaggagaacctcagtctgacgacattgaagctagccgaatgaagcgagcagctgcaaagcatctaatagaacgctactaccaccagttaactgagggctgtggaaatgaagcctgcacgaatgagttttgtgcttcctgtccaacttttcttcgtatggataataatgcagcagctattaaagccctcgagctttataagattaatgcaaaactctgtgatcctcatccctccaagaaaggagcaagctcagcttaccttgagaactcgaaaggtgcccccaacaactcctgctctgagataaaaatgaacaagaaaggcgctagaattgattttaaagatgtgacttacttaacagaagagaaggtatatgaaattcttgaattatgtagagaaagagaggattattcccctttaatccgtgttattggaagagttttttctagtgctgaggcattggtacagagcttccggaaagttaaacaacacaccaaggaagaactgaaatctcttcaagcaaaagatgaagacaaagatgaagatgaaaaggaaaaagctgcatgttctgctgctgctatggaagaagactcagaagcatcttcctcaaggataggtgatagctcacagggagacaacaatttgcaaaaattaggccctgatgatgtgtctgtggatattgatgccattagaagggtctacaccagattgctctctaatgaaaaaattgaaactgcctttctcaatgcacttgtatatttgtcacctaacgtggaatgtgacttgacgtatcacaatgtatactctcgagatcctaattatctgaatttgttcattatcgtaatggagaatagaaatctccacagtcctgaatatctggaaatggctttgccattattttgcaaagcgatgagcaagctaccccttgcagcccaaggaaaactgatcagactgtggtctaaatacaatgcagaccagattcggagaatgatggagacatttcagcaacttattacttataaagtcataagcaatgaatttaacagtcgaaatctagtgaatgatgatgatgccattgttgctgcttcgaagtgcttgaaaatggtttactatgcaaatgtagtgggaggggaagtggacacaaatcacaatgaagaagatgatgaagagcccatccctgagtccagcgagctgacacttcaggaacttttgggagaagaaagaagaaacaagaaaggtcctcgagtggaccccctggaaactgaacttggtgttaaaaccctggattgtcgaaaaccacttatcccttttgaagagtttattaatgaaccactgaatgaggttctagaaatggataaagattatacttttttcaaagtagaaacagagaacaaattctcttttatgacatgtccctttatattgaatgctgtcacaaagaatttgggattatattatgacaatagaattcgcatgtacagtgaacgaagaatcactgttctctacagcttagttcaaggacagcagttgaatccatatttgagactcaaagttagacgtgaccatatcatagatgatgcacttgtccggctagagatgatcgctatggaaaatcctgcagacttgaagaagcagttgtatgtggaatttgaaggagaacaaggagttgatgagggaggtgtttccaaagaattttttcagctggttgtggaggaaatcttcaatccagatattggtatgttcacatacgatgaatctacaaaattgttttggtttaatccatcttcttttgaaactgagggtcagtttactctgattggcatagtactgggtctggctatttacaataactgtatactggatgtacattttcccatggttgtctacaggaagctaatggggaaaaaaggaacttttcgtgacttgggagactctcacccagttctatatcagagtttaaaagatttattggagtatgaagggaatgtggaagatgacatgatgatcactttccagatatcacagacagatctttttggtaacccaatgatgtatgatctaaaggaaaatggtgataaaattccaattacaaatgaaaacaggaaggaatttgtcaatctttattctgactacattctcaataaatcagtagaaaaacagttcaaggcttttcggagaggttttcatatggtgaccaatgaatctcccttaaagtacttattcagaccagaagaaattgaattgcttatatgtggaagccggaatctagatttccaagcactagaagaaactacagaatatgacggtggctataccagggactctgttctgattagggagttctgggaaatcgttcattcatttacagatgaacagaaaagactcttcttgcagtttacaacgggcacagacagagcacctgtgggaggactaggaaaattaaagatgattatagccaaaaatggcccagacacagaaaggttacctacatctcatacttgctttaatgtgcttttacttccggaatactcaagcaaagaaaaacttaaagagagattgttgaaggccatcacgtatgccaaaggatttggcatgctgtaaaacaaaacaaaacaaaataaaacaaaaaaaaggaaggaaaaaaaaagaaaaaatttaaaaaattttaaaaatataacgagggataaatttttggtggtgatagtgtcccagtacaaaaaggctgtaagatagtcaaccacagtagtcacctatgtctgtgcctcccttctttattggggacatgtgggctggaacagcagatttcagctacatatatgaacaaatcctttattattattataattatttttttgcgtgaaagtgttacatattctttcacttgtatgtacagagaggtttttctgaatatttattttaagggttaaatcacttttgcttgtgtttattactgcttgaggttgagccttttgagtatttaaaaaatatataccaacagaactactctcccaaggaaaatattgccaccatttgtagaccacgtaaccttcaagtatgtgctacttttttgtccctgtatctaactcaaatcaggaactgtattttttttaatgatttgcttttgaaacttgaagtcttgaaaacagtgtgatgcaattactgctgttctagcccccaaagagttttctgtgcaaaatcttgagaatcaatcaataaagaaagatggaaggaagggagaaattggaatgttttaactgcagccctcagaactttagtaacagcacaacaaattaaaaacaaaaacaactcatgccacagtatgtcgtcttcatgtgtcttgcaatgaactgtttcagtagccaatcctctttcttagtatatgaaaggacagggatttttgttcttgttgttctcgttgttgttttaagtttactggggaaagtgcatttggccaaatgaaatggtagtcaagcctattgcaacaaagttaggaagtttgttgtttgtttattataaacaaaaagcatgtgaaagtgcacttaagatagagtttttattaattacttacttattacctagattttaaatagacaatccaaagtctccccttcgtgttgccatcatcttgttgaatcagccattttatcgaggcacgtgatcagtgttgcaacataatgaaaaagatggctactgtgccttgtgttacttaatcatacagtaagctgacctggaaatgaatgaaactattactcctaagaattacattgtatagccccacagattaaatttaattaattaattcaaaacatgttaaacgttactttcatgtactatggaaaagtacaagtaggtttacattactgatttccagaagtaagtagtttcccctttcctagtcttctgtgtatgtgatgttgttaatttcttttattgcattataaaataaaaggattatgtatttttaactaaggtgagacattgatatatccttttgctacaagctatagctaatgtgctgagcttgtgccttggtgattgattgattgattgactgattgttttaactgattactgtagatcaacctgatgatttgtttgtttgaaattggcaggaaaaatgcagctttcaaatcattggggggagaaaaaggatgtctttcaggattattttaattaatttttttcataattgagacagaactgtttgttatgtaccataatgctaaataaaactgtggcacttttcaccataatttaatttagtggaaaaagaagacaatgctttccatattgtgataaggtaacatggggtttttctgggccagcctttagaacactgttagggtacatacgctaccttgatgaaagggaccttcgtgcaactgtagtcatcttaaaggcttctcatccactgtgcttcttaatgtgtaattaaagtgaggagaaattaaatactctgagggcgttttatataataaattcgtgaaga

SEQ ID NO: 10 - protein, 875 aa, Homo sapiens
MEKLHQCYWKSGEPQSDDIEASRMKRAAAKHLIERYYHQLTEGCGNEACTNEFCASCPTFLRMDNNAAAIKALELYKINAKLCDPHPSKKGASSAYLENSKGAPNNSCSEIKMNKKGARIDFKDVTYLTEEKVYEILELCREREDYSPLIRVIGRVFSSAEALVQSFRKVKQHTKEELKSLQAKDEDKDEDEKEKAACSAAAMEEDSEASSSRIGDSSQGDNNLQKLGPDDVSVDIDAIRRVYTRLLSNEKIETAFLNALVYLSPNVECDLTYHNVYSRDPNYLNLFIIVMENRNLHSPEYLEMALPLFCKAMSKLPLAAQGKLIRLWSKYNADQIRRMMETFQQLITYKVISNEFNSRNLVNDDDAIVAASKCLKMVYYANVVGGEVDTNHNEEDDEEPIPESSELTLQELLGEERRNKKGPRVDPLETELGVKTLDCRKPLIPFEEFINEPLNEVLEMDKDYTFFKVETENKFSFMTCPFILNAVTKNLGLYYDNRIRMYSERRITVLYSLVQGQQLNPYLRLKVRRDHIIDDALVRLEMIAMENPADLKKQLYVEFEGEQGVDEGGVSKEFFQLVVEEIFNPDIGMFTYDESTKLFWFNPSSFETEGQFTLIGIVLGLAIYNNCILDVHFPMVVYRKLMGKKGTFRDLGDSHPVLYQSLKDLLEYEGNVEDDMMITFQISQTDLFGNPMMYDLKENGDKIPITNENRKEFVNLYSDYILNKSVEKQFKAFRRGFHMVTNESPLKYLFRPEEIELLICGSRNLDFQALEETTEYDGGYTRDSVLIREFWEIVHSFTDEQKRLFLQFTTGTDRAPVGGLGKLKMIIAKNGPDTERLPTSHTCFNVLLLPEYSSKEKLKERLLKAITYAKGFGML

(19) United States
(12) Patent Application Publication
(10) Pub. No.: US 2024/0216536 A1
(43) Pub. Date: Jul. 4, 2024

(54) SECRETED UBE3A FOR TREATMENT OF NEUROLOGICAL DISORDERS

(71) Applicant: University of South Florida, Tampa, FL (US)

(72) Inventor: Kevin Ron Nash, Temple Terrace, FL (US)

(21) Appl. No.: 18/392,696

(22) Filed: Dec. 21, 2023

Related U.S. Application Data
(63) Continuation of application No. PCT/US2022/035166, filed on Jun. 27, 2022.
(60) Provisional application No. 63/202,835, filed on Jun. 25, 2021.

(51) Int. Cl. A61K 48/00; A61K 9/00; A61K 38/53; A61P 25/28; C12N 15/86

(52) U.S. Cl. CPC A61K 48/005; A61K 9/0085; A61K 38/53; A61K 48/0075; A61P 25/28; C12N 15/86; C12N 2750/14143; C12Y 603/02019

(57) ABSTRACT

A novel vector, composition and method of treating a UBE3A deficiency disease is presented. A novel UBE3A vector construct was generated with an additional secretion sequence to allow the secretion from cells. This secreted only E6AP protein maintains its presence outside the cell and is capable of diffusing to greater distances to cover more of the brain and rescue disease pathology.

### CROSS REFERENCE TO RELATED APPLICATIONS

This application is a continuation of and claims priority to International Patent Application Serial No. PCT/US2022/035166, entitled “Secreted UBE3A for the Treatment of Neurological Disorders”, filed Jun. 27, 2022 which is a nonprovisional of and claims priority to U.S. Provisional Patent Application No. 63/202,835, entitled “Secreted UBE3A for the Treatment of Neurological Disorders”, filed Jun. 25, 2021, the contents of which are hereby incorporated by reference into this disclosure.

### SEQUENCE LISTING

The sequence listing entitled “Secreted UBE3A for Treatment of Neurological Disorders” in XML format, created on Nov. 21, 2023 and being 35,000 bytes in size, is hereby incorporated by reference into this disclosure.

### FIELD OF INVENTION

This invention relates to treatment of Angelman syndrome. More specifically, the present invention provides therapeutic methods and compositions for treating Angelman syndrome.

### BACKGROUND OF THE INVENTION

Angelman syndrome (AS) is a genetic disorder affecting neurons, estimated to effect about one in every 15,000 births (Clayton-Smith, Clinical research on Angelman syndrome in the United Kingdom: observations on 82 affected individuals. Am J Med Genet. 1993 Apr. 1; 46(1):12-5), though the actual number of diagnosed AS cases is greater likely due to misdiagnosis.

Angelman syndrome is a continuum of impairment, which presents with delayed and reduced intellectual and developmental advancement, most notably regarding language and motor skills. In particular, AS is defined by little or no verbal communication, with some non-verbal communication, ataxia, and disposition that includes frequent laughing and smiling and excitable movement. Some symptoms include severe developmental delay, severe speech impairment, gait/limb movement disorder, altered personality (unusually happy and energetic, seizures and sleep abnormalities, and abnormal EEG.

More advanced cases result in severe mental retardation, seizures that may be difficult to control that typically begin before or by three years of age, frequent laughter (Nicholls, New insights reveal complex mechanisms involved in genomic imprinting. Am J Hum Genet. 1994 May; 54(5):733-40), miroencephaly, and abnormal EEG. In severe cases, patients may not develop language or may only have use of 5-10 words. Movement is commonly jerky, and walking commonly is associated with hand flapping and a stiff-gait. The patients are commonly epileptic, especially earlier in life, and suffer from sleep apnea, commonly only sleeping for 5 hours at a time. They are social and desire human contact. In some cases, skin and eyes may have little or no pigment, they may possess sucking and swallowing problems, sensitivity to heat, and a fixation to water bodies. Studies in UBE3A-deficient mice show disturbances in long-term synaptic plasticity. There are currently no cures for Angelman syndrome, and treatment is palliative. For example, anticonvulsant medication is used to reduce epileptic seizures, and speech and physical therapy are used to improve language and motor skills.

The gene UBE3A is responsible for AS and it is unique in that it is one of a small family of human imprinted genes. UBE3A, found on chromosome 15, encodes for the homologous to E6AP C terminus (HECT) protein (E6-associated protein (E6AP) (Kishino, et al., UBE3A/E6-AP mutations cause Angelman syndrome. Nat Gen. 1997 Jan. 15.15(1):70-3). UBE3A undergoes spatially-defined maternal imprinting in the brain; thus, the paternal copy is silenced via DNA methylation (Albrecht, et al., Imprinted expression of the murine Angelman syndrome gene, Ube3a, in hippocampal and Purkinje neurons. Nat Genet. 1997 September; 17(1):75-8). As such, only the maternal copy is active, the paternal chromosome having little or no effect on the proteosome of the neurons in that region of the brain. Inactivation, translocation, or deletion of portions of chromosome 15 therefore results in uncompensated loss of function. Some studies suggest improper E3-AP protein levels alter neurite contact in Angelman syndrome patients (Tonazzini, et al., Impaired neurite contract guidance in ubiquitin ligase E3a (Ube3a)-deficient hippocampal neurons on nanostructured substrates. Adv Healthc Mater. 2016 April; 5(7):850-62).

The majority of Angelman's syndrome cases (70%) occur through a de novo deletion of around 4 Mb from 15q11-q13 of the maternal chromosome which incorporates the UBE3A gene (Kaplan, et al., Clinical heterogeneity associated with deletions in the long arm of chromosome 15: report of 3 new cases and their possible significance. Am J Med Genet. 1987 September; 28(1):45-53), but it can also occur as a result of abnormal methylation of the maternal copy, preventing its expression (Buiting, et al., Inherited microdeletions in the Angelman and Prader-Willi syndromes define an imprinting centre on human chromosome 15. Nat Genet. 1995 April; 9(4):395-400; Gabriel, et al., A transgene insertion creating a heritable chromosome deletion mouse model of Prader-Willi and Angelman syndrome. Proc Natl Acad Sci U.S.A. 1999 August; 96(16):9258-63) or uniparental disomy in which two copies of the paternal gene are inherited (Knoll, et al., Angelman and Prader-Willi syndromes share a common chromosome 15 deletion but differ in parental origin of the deletion. Am J Med Genet. 1989 Fed; 32(2):285-90; Malcolm, et al., Uniparental paternal disomy in Angelman's syndrome. Lancet. 1991 Mar. 23; 337(8743):694-7). The remaining AS cases arise through various UBE3A mutations of the maternal chromosome or they are diagnosed without a genetic cause (12-15UBE3A codes for the E6-associated protein (E6-AP) ubiquitin ligase. E6-AP is an E3 ubiquitin ligase, therefore it exhibits specificity for its protein targets, which include the tumor suppressor molecule p53 (Huibregtse, et al., A cellular protein mediates association of p53 with the E6 oncoprotein of human papillomavirus types 16 or18. EMBO J. 1991 December; 10(13):4129-35), a human homologue to the yeast DNA repair protein Rad23 (Kumar, et al., Identification of HHR23A as a substrate for E6-associated protein-mediated ubiquitination. J Biol Chem. 1999 Jun. 25; 274(26):18785-92), E6-AP itself, and Arc, the most recently identified target (Nuber, et al., The ubiquitin-protein ligase E6-associated protein (E6-AP) serves as its own substrate. Eur J Biochem. 1998 Jun. 15; 254(3):643-9; Greer, et al., The Angelman Syndrome protein Ube3A regulates synapse Development by ubiquitinating arc. Cell. 2010 Mar. 5; 140(5): 704-16).

Mild cases are likely due to a mutation in the UBE3A gene at chromosome 15q11-13, which encodes for E6-AP ubiquitin ligase protein of the ubiquitin pathway, and more severe cases resulting from larger deletions of chromosome 15. Commonly, the loss of the UBE3A gene in the hippocampus and cerebellum result in Angelman syndrome, though single loss-of-function mutations can also result in the disorder.

For treatment purposes one can consider AS a monogenic disorder, which is evidenced by AS patients with disease causing point mutations in UBE3A. This suggests gene replacement therapy as a promising avenue of treatment for this monogenic disorder. Investigations into gene therapy-based treatments for neurological disorders have been increasing for several years, especially for treatment of monogenic disorders. The recent FDA approval of gene therapy approaches for RPE65 mutation-associated retinal dystrophy and type I spinal muscular atrophy supports the progression for other monogenic disorders like AS. This makes UBE3A a clear target for a disease-modifying treatment. The inventors have previously shown recovery of deficits in the mouse model of AS using recombinant adeno-associated viral vectors (rAAV) expressing mouse Ube3a [Daily et al. 2011]. While there is a clear potential for rAAV-mediated gene therapy, the primary challenge is getting rAAV to transduce a majority of UBE3A-deficient neurons, while minimizing high titer and the number of injections.

Deficiencies in Ube3a are also linked in Huntington's disease (Maheshwari, et al., Deficiency of Ube3a in Huntington's disease mice brain increases aggregate load and accelerates disease pathology. Hum Mol Genet. 2014 Dec. 1; 23(23):6235-45). Matentzoglu noted E6-AP possesses non-E3 activity related to hormone signaling (Matentzoglu, EP 2,724,721 A1). There is some association of UBE3A being implicated in Alzheimer's disease as well. (Olabarria M. et al., Dysfunction of the ubiquitin ligase E3A Ube3A/E6-AP contributes to synaptic pathology in Alzheimer's disease, Commun Biol, 2019, 2:111). AS patients have significantly elevated plasma A040 and 42 compared to age-matched control individuals. (Erickson et al. Analysis of peripheral amyloid precursor protein in Angelman Syndrome. Am J Med Genet A. 2016; 170(9):2334-7). Aberrant APP regulation has been reported in models of chromosome 15q11-13 duplication (Dup15q), in which UBE3A gene expression is increased 1.5- to 2-fold, which demonstrated that APP levels were decreased 10-fold relative to control samples. (Baron et al. Genomic and functional profiling of duplicated chromosome 15 cell lines reveal regulatory alterations in UBE3A-associated ubiquitin-proteasome pathway processes. Hum Mol Genet. 2006; 15(6):853-69).

As such, administration of steroids, such as androgens, estrogens, and glucocorticoids, was used for treating various E6-AP disorders, including Angelman syndrome, autism, epilepsy, Prader-Willi syndrome, cervical cancer, fragile X syndrome, and Rett syndrome. Philpot suggested using a topoisomerase inhibitor to demethylate silenced genes thereby correcting for deficiencies in Ube3A (Philpot, et al., P.G. Pub. US 2013/0317018 A1). However, work in the field, and proposed therapeutics, do not address the underlying disorder, as in the use of steroids, or may result in other disorders, such as autism, where demethylation compounds are used.

Nash & Weeber (WO 2016/179584) demonstrated that recombinant adeno-associated virus (rAAV) vectors can be an effective method for gene delivery in mouse models. However, only a small population of neurons are successfully transduced and thus express the protein, preventing global distribution of the protein in the brain as needed for efficacious therapy. As such, what is needed is a therapeutic that provides for supplementation of Ube3a protein throughout the entire brain.

### SUMMARY OF THE INVENTION

In an embodiment, a UBE3A vector was formed using a transcription initiation sequence, and a UBE construct disposed downstream of the transcription initiation sequence. The UBE construct is formed of a UBE3A sequence and a secretion sequence. Nonlimiting examples of the UBE3A sequence include a cDNA of SEQ ID NO: 1, SEQ ID NO: 2, SEQ ID NO: 7, a cDNA of SEQ ID NO: 8, SEQ ID NO: 9, a cDNA of SEQ ID NO: 10 or a homologous sequence thereof. Nonlimiting examples of the secretion sequence include GDNF (SEQ ID NO: 3), insulin (SEQ ID NO: 5), IgK (SEQ ID NO: 6), or a homologous sequence thereof. In some variations, the secretion sequence is disposed upstream of the UBE3A sequence.

In some variations of the invention, the transcription initiation sequence is a cytomegalovirus chicken-beta actin hybrid promoter, or human ubiquitin c promoter. The invention optionally includes an enhancer sequence. A nonlimiting example of the enhancer sequence is a cytomegalovirus immediate-early enhancer sequence disposed upstream of the transcription initiation sequence. The vector optionally also includes a woodchuck hepatitis post-transcriptional regulatory element.

In variations, the vector is inserted into a plasmid, such as a recombinant adeno-associated virus serotype 2-based plasmid. In specific variations, the recombinant adeno-associated virus serotype 2-based plasmid lacks DNA integration elements. A nonlimiting example of the recombinant adeno-associated virus serotype 2-based plasmid is a pTR plasmid.

Also presented is a method of treating a UBE3A deficiency disease such as Angelman syndrome, Prader-Willi syndrome, or Huntington's disease, by administering a therapeutically effective amount of UBE3A vector, as described previously, to the brain of a patient in order to correct the UBE3A deficiency. The vector may be administered by injection into the brain, such as by intrahippocampal or intraventricular injection or a combination thereof. In some instances, the vector may be injected bilaterally. Exemplary dosages can range between about 5.55×10¹¹ to 2.86×10¹² genomes/g brain mass or between 2.0×10¹³ to 4.0×10¹³ vg/ml.

A composition for use in treating a UBE3A deficiency disease is also presented. The composition may be comprised of a UBE3A vector as described above, and a pharmaceutically acceptable carrier. In some instances, the pharmaceutically acceptable carrier can be a blood brain barrier permeabilizer such as mannitol.

### BRIEF DESCRIPTION OF THE DRAWINGS

For a fuller understanding of the invention, reference should be made to the following detailed description, taken in connection with the accompanying drawings, in which:

FIG. 1A-H are a series of images depicting UBE3A is located in the cerebrospinal fluid and extracellular space of the brain. a) Representative image of western blot of individual rat CSF samples using anti-UBE3A antibody. AS rats have a significant reduction in UBE3A compared to WT rats. b) Representative image of individual mouse CSF western blot using anti-Ube3a. AS mice have a significant deficit in CSF UBE3A compared to WT littermates. c) Representative image of individual neuro-typical human CSF western blot. d) Representative image of UBE3A dialysate western blot demonstrating presence of UBE3A in the hippocampal extracellular space. e) Quantitation of anti-UBE3A in rat CSF relative to total protein levels by revert (WT n=9 (3M/6F), AS n=9 (5M/4F), t(16)=7.366, p<0.0001). f) Quantitation of anti-UBE3A in mouse CSF relative to total protein levels by revert (WT n=5 (2M/3F), AS n=6 (4M/2F), t(9)=4.898, p<0.0001). g) Amount of UBE3A protein present in human CSF (Neuro-typical n=5) using a standard curve of recombinant human UBE3A protein (Boston Biochem). h) Quantitation of anti-UBE3A in rat hippocampal dialysate relative to total protein levels by revert (WT n=25 (14M/10F), AS n=4 (3M/1F), t(27)=4.4.73, p<0.0001). Error bars represent SEM.

FIG. 2 is an image depicting rat isoform 1 amino acid sequence from NCBI (GI:394025712)(SEQ ID NO: 1). Peptide sequences that were identified from mass spectrometry analysis are indicated by a line under the protein sequence. 22 different peptides covering a significant proportion of the protein were identified. Bold M indicates start of isoform 3.

FIG. 3A-E are a series of images depicting UBE3A maintains its catalytic activity within the CSF and hippocampal extracellular space. a) Representative image of rat CSF ubiquitination assay western blot at 3 different time points (min) (n=12, 10M/2F); a-UBE3A Sigma, a-S5A Boston Biochem) b) Representative image of hippocampal dialysate ubiquitination assay western blot (n=2, 1M/1F). c) Representative image of rat WT dialysate with UBE3A immuno-precipitated (n=1M). d) Representative image of AS rat dialysate ubiquitination assay western blot at 3 different time points (n=2, 1M/1F). e) Quantification of S5A 40 kDa band intensity, expressed as percentage of zero time value, for each assay.

FIG. 4A-C are a series of images depicting extracellular UBE3A undergoes activity dependent regulation following fear conditioning. a) Western blot quantification of baseline measurements from WT rat hippocampal dialysate, UBE3A band intensity normalized to revert total protein stain. Representative western blots of UBE3A and revert total protein stain (n=7 3M/4F) b) Control animals placed in the fear conditioning chamber with no shock or tone applied. Western blot quantification of UBE3A normalized to revert total protein stain and representative western blots (No shock: 2.0 hr t(15)=3.028; 2.5 hr t(15)=2.276; n=9 6M/3F). c) Animals placed in fear conditioning chamber received shock following 1.5 hour habituation to microdialysis chamber. Western blot quantification of hippocampal dialysate UBE3A levels normalized to revert total protein stain and representative western blots (Shock: 4.0 hr t(14)=2.689; 4.5 hr t(13)=2.436; 5.0 hr t(14)=2.632; 5.5 hr t(14)=2.162; 6.0 hr t(13)=2.523; 6.5 hr t(14)=2.365; 7.0 hr t(14)=3.292; 7.5 hr t(14)=3.696; n=8 5M/3F) *p<0.05, #baseline samples averaged for comparison to time points).

FIG. 5A-B are a series of images depicting input/output curve reveals reduced hippocampal functional connectivity in AS maternal Ube3a deficient rats. a) Fiber volley amplitude with increasing stimulus intensity. b) fEPSP slope during input/output recordings with increasing stimulus intensity.

FIG. 5C is an image depicting input/output curve reveals reduced hippocampal functional connectivity in AS maternal Ube3a deficient rats. c) input/output curve comparing slopes of fEPSPs and fiber volley. (WT n=6 slices n=12, AS n=11 slices n=18).

FIG. 6A-B are a series of images depicting AS rats have a deficit in hippocampal CA3-CA1 μLTP. a) Slices were maintained at 30.2° C. and LTP was induced with 2 trains of 100 Hz stimulation for 1 sec separated by 20 sec. All results are graphed as the percentage of potentiation standardized to the baseline recording. Dashed line represents 100% mark of baseline synaptic responses. Data represent mean±SEM. (WT n=10 slices n=26, AS n=8 slices n=20). b) Averaged slopes for last 10 minutes (50-60 min) after HFS (t(10)=85.94, ***p<0.0001).

FIG. 7A-B are a series of images depicting exogenous recombinant human UBE3A protein application recovers CA3-CA1 μLTP deficits in AS rats. a) Slices were maintained at 30.2° C. and LTP was induced with 2 trains of 100 Hz stimulation for 1 sec separated by 20 sec following incubation with exogenous UBE3A protein (30 min). All results are graphed as the percentage of potentiation standardized to the baseline recording. Dashed line represents 100% mark of baseline synaptic responses. Data represent mean±SEM (AS n=8 slices n=20, AS with UBE3A protein n=7 slices n=15). b) Averaged slopes for last 10 minutes (50-60 min) after HFS (t(10)=21.41, ***p<0.0001).

FIG. 8A-B are a series of images depicting input/output curve with exogenous supplementation of UBE3A protein. a) Fiber volley amplitude with increasing stimulus intensity. b) fEPSP slope during input/output recordings with increasing stimulus intensity.

FIG. 8C is an image depicting input/output curve with exogenous supplementation of UBE3A protein. c) input/output curve comparing slopes of fEPSPs and fiber volley. (WT n=10 (6F/4M) slices n=12, AS n=8 (6F/2M) slices n=18, WT with UBE3A protein n=6 (4F/2M) slices=24, AS with UBE3A protein n=7 (4F/3M) slices=17).

FIG. 9A-B are a series of images depicting exogenous recombinant human UBE3A protein injection into AS rat hippocampi recovers hippocampal dependent learning and memory but not after heat inactivation. A) Training for fear conditioning shows no differences between groups. B) Contextual fear conditioning test shows rescue of AS deficit with UBE3A protein injection (F(3, 54)=12.61, P<0.0001).

FIG. 9C is an image depicting exogenous recombinant human UBE3A protein injection into AS rat hippocampi recovers hippocampal dependent learning and memory but not after heat inactivation. C) Cued fear conditioning shows rescue with UBE3A protein injection (F(3,54)=8.29, p=0.0001). AS mock injected (n=13 (7F/6M)); AS UBE3A protein injected (n=12 (5F/7M)), AS HI-UBE3A protein injected (n=13 (6F/7M)) and wild type (n=20 (9F/11M)). No significant differences were found between sexes. Statistics: One Way ANOVA, Tukey post-hoc test,*p<0.05, **p<0.005, ***p=0.0001, ****p<0.0001.

FIG. 10A-B is a series of images depicting generation of a secreted form of UBE3A. A) Diagram of AAV vector constructs. Using the human isoform 1 UBE3A coding sequence STUB was created by attaching a GDNF signal for secretion and a TAT sequence for reuptake to the N-terminus. Both hUBE3A and STUB were cloned into pTR expression vectors with the CBA (or CAG) promoter. B) Western blot using anti-HA antibody of cell culture media collected from HEK293 cells transfected with pTR-hUBE3A-HA or pTR-STUB-HA showed expression and secretion of the STUB-HA protein into the cell culture media.

FIG. 11A-F are a series of images depicting hippocampal expression of UBE3A and STUB from Unilateral Hippocampal injected AS rats verified by immunohistochemistry To examine the ability of various rAAVs to express UBE3A in vivo, AS rats were unilaterally injected into the hippocampus with vectors containing STUB or UBE3A. After allowing 6 weeks for protein expression, tissue was collected and probed for UBE3A. Representative images are shown. A) UBE3A has a strong signal in the injected ipsilateral hemisphere B) There is no staining in the uninjected contralateral hemisphere of UBE3A animals. C) STUB has a diffuse expression in the injected hemisphere D) The uninjected hemisphere from STUB animals have no staining. E) Wild type hippocampus stained for UBE3A. Scale bar=200 μm. F) Quantification of staining represented as percentage area of positive staining. WT=wild type, Co.=contralateral, Ip.=ipsilateral.

FIG. 12A-B are a series of images depicting STUB Retains Function in Treated AS Mice. A) Hippocampal LTP testing demonstrates that STUB recovers the LTP deficit present in AS mice. Data shown as mean and standard error of the mean. B) An average of the final 10 minutes of electrophysiological testing shows a recovery of LTP to wildtype levels. ANOVA results indicate a significant difference between groups (F (2, 15)=267.7). Tukey's multiple comparison test found that the average of EPSP slopes was significantly different between the GFP group and STUB group and the GFP group and the wildtype group. No difference was found between the STUB and wildtype groups. Data shown as mean and standard error of the mean. ****p<0.0001.

FIG. 13A-D are a series of images depicting Hippocampal long-term potentiation in unilaterally injected AS rats is rescued in both contralateral and ipsilateral hemispheres by STUB treatment. A) HPC LTP testing in animals treated by unilateral HPC injection of STUB. (STUB uninjected n=6 slices, 3 animals; STUB injected n=5 slices, 3 animals; PBS n=7 slices, 3 animals) C) HPC LTP testing in animals treated by unilateral HPC injection of UBE3A. (UBE3A uninjected n=7 slices, 4 animals; UBE3A injected n=5 slices, 3 animals; PBS n=7 slices, 3 animals) B and D) Quantification of the final 10 minutes of each experiment. ANOVA results: F (2, 12)=9.442, p=0.0034. Statistical significance as tested by Tukey's test shown as asterisks.

FIG. 14A-B are a series of images depicting electrophysiological deficits are recovered in ICV STUB injected AS rats. A) Hippocampal LTP deficit shows recovery after treatment with STUB via ICV injection with a partial recovery shown in animals treated with an identical injection of UBE3A. B) Quantification of the last 10 minutes of electrophysiology recordings shows a recovery with the STUB treatment and a partial recovery with UBE3A treatment. (wildtype n=10 slices, 4 animals; STUB injected n=11 slices, 4 animals; UBE3A injected n=11 slices, 4 animals, UBE3A injected n=11 slices, 5 animals) ANOVA results: F (3, 16)=88.96, p<0.0001. Statistical significance as tested by Tukey's test shown as asterisks.

FIG. 15A-C are a series of images depicting STUB Improves the Behavioral Deficits Present in the AS Rat. ICV treatment of AS rats with STUB shows an improvement in 3 different behavioral tests. A) ICV UBE3A treatment shows no improvement in motor function as measured by Rotarod. STUB treatments results in an improvement in motor learning on day two to Wildtype levels. The UBE3A group and PBS groups demonstrate a lack of learning on day 2. A reduced latency to fall was found between UBE3A and PBS groups on trials 7 and 8. Repeated measures ANOVA performed (F (21, 434)=1.679 p=0.031). B) The hind limb clasping phenotype is recovered with ICV STUB treatment but not UBE3A treatment. ANOVA results indicate a difference between groups. (F (3, 52)=7.242). Tukey's multiple comparison test found a difference between wildtype and both UBE3A (p=0.017) and PBS (p=0.002). The STUB group had a score equivalent to wildtype. C) ANOVA results indicate a difference between groups. (F (3, 47)=3.121) STUB and UBE3A both result in a partial improvement of associated learning as tested by contextual fear conditioning; however, the magnitude of improvement is greater with STUB treatment. Tukey's multiple comparison test found a difference between PBS and wildtype groups (p=0.0222) but no difference between other groups. *p<0.05; **p<0.01 (hind limb clasp: wildtype n=14, STUB n=13, UBE3A n=15, PBS n=14; Rotarod: wildtype n=13, STUB n=18, UBE3A n=17, PBS n=18; fear conditioning: wildtype n=13, STUB n=13, UBE3A n=12, PBS n=13).

FIG. 16 is an image of the hSUB vector.

FIG. 17A-B is a series of images depicting AS rats have a significant deficit in chemically induced LTD. a) Following baseline measurements, LTD was induced by (S)3,5-DHPG (10 μM) for 10 min and fEPSPs were recorded (average of 6 traces). b) Averaged slopes of fEPSPs for the last 10 min (40-50 min) after LTD induction (WT n=6 slices n=12, AS n=6 slices n=11) t(10)=22.62, ****p<0.0001.

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments may be utilized, and structural changes may be made without departing from the scope of the invention. The following description is not intended to limit the scope of the present description disclosed herein.

As used herein, the singular forms “a,” “an” and “the” include plural referents unless the context clearly dictates otherwise. Thus, for example, reference to “a polypeptide” includes a mixture of two or more polypeptides and the like.

Unless otherwise defined, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are described herein. All publications mentioned herein are incorporated herein by reference in their entirety to disclose and describe the methods and/or materials in connection with which the publications are cited. It is understood that the present disclosure supersedes any disclosure of an incorporated publication to the extent there is a contradiction.

All numerical designations, such as pH, temperature, time, concentration, and molecular weight, including ranges, are approximations which are varied up or down by increments of 1.0 or 0.1, as appropriate. It is to be understood, even if it is not always explicitly stated that all numerical designations are preceded by the term “about”. It is also to be understood, even if it is not always explicitly stated, that the reagents described herein are merely exemplary and that equivalents of such are known in the art and can be substituted for the reagents explicitly stated herein.

As used herein, the term “comprising” is intended to mean that the products, compositions and methods include the referenced components or steps, but not excluding others. “Consisting essentially of” when used to define products, compositions and methods, shall mean excluding other components or steps of any essential significance. Thus, a composition consisting essentially of the recited components would not exclude trace contaminants and pharmaceutically acceptable carriers. “Consisting of” shall mean excluding more than trace elements of other components or steps.

As used in the specification and claims, the singular form “a”, “an” and “the” include plural references unless the context clearly dictates otherwise. For example, the term “a vector” includes a plurality of vectors.

As used herein, “about” means approximately or nearly and in the context of a numerical value or range set forth means±15% of the numerical.

“Adeno-associated virus (AAV) vector” as used herein refers to an adeno-associated virus vector that can be engineered for specific functionality in gene therapy. In some instances, the AAV can be a recombinant adeno-associated virus vector, denoted rAAV.

Any suitable AAV known in the art can be used, including, but not limited to, AAV2, AAV9, AAV5, AAV1 and AAV4.

“Administration” or “administering” is used to describe the process in which compounds of the present invention, alone or in combination with other compounds, are delivered to a patient. The composition may be administered in various ways including injection into the central nervous system including the brain, including but not limited to, intrastriatal, intrahippocampal, ventral tegmental area (VTA) injection, intracerebral, intracerebroventricular, intracerebellar, intramedullary, intranigral, intraventricular, intracisternal, intracranial, intraparenchymal including spinal cord and brain stem; oral; parenteral (referring to intravenous and intraarterial and other appropriate parenteral routes); intrathecal; intramuscular; subcutaneous; rectal; and nasal, among others. Each of these conditions may be readily treated using other administration routes of compounds of the present invention to treat a disease or condition.

“Treatment” or “treating” as used herein refers to any of: the alleviation, amelioration, elimination and/or stabilization of a symptom, as well as delay in progression of a symptom of a particular disorder. For example, “treatment” of a neurodegenerative disease may include any one or more of the following: amelioration and/or elimination of one or more symptoms associated with the neurodegenerative disease, reduction of one or more symptoms of the neurodegenerative disease, stabilization of symptoms of the neurodegenerative disease, and delay in progression of one or more symptoms of the neurodegenerative disease.

“Prevention” or “preventing” as used herein refers to any of: halting the effects of the neurodegenerative disease, reducing the effects of the neurodegenerative disease, reducing the incidence of the neurodegenerative disease, reducing the development of the neurodegenerative disease, delaying the onset of symptoms of the neurodegenerative disease, increasing the time to onset of symptoms of the neurodegenerative disease, and reducing the risk of development of the neurodegenerative disease.

The pharmaceutical compositions of the subject invention can be formulated according to known methods for preparing pharmaceutically useful compositions. Furthermore, as used herein, the phrase “pharmaceutically acceptable carrier” means any of the standard pharmaceutically acceptable carriers. The pharmaceutically acceptable carrier can include diluents, adjuvants, and vehicles, as well as implant carriers, and inert, non-toxic solid or liquid fillers, diluents, or encapsulating material that does not react with the active ingredients of the invention. Examples include, but are not limited to, phosphate buffered saline, physiological saline, water, and emulsions, such as oil/water emulsions. In some embodiments, the pharmaceutically acceptable carrier can be a blood brain permeabilizer including, but not limited to, mannitol. The carrier can be a solvent or dispersing medium containing, for example, ethanol, polyol (for example, glycerol, propylene glycol, liquid polyethylene glycol, and the like), suitable mixtures thereof, and vegetable oils. Formulations are described in a number of sources that are well known and readily available to those skilled in the art. For example, Remington's Pharmaceutical Sciences (Martin E W [1995] Easton Pennsylvania, Mack Publishing Company, 19^{th} ed.) describes formulations which can be used in connection with the subject invention.

As used herein “animal” means a multicellular, eukaryotic organism classified in the kingdom Animalia or Metazoa. The term includes, but is not limited to, mammals. Non-limiting examples include humans, rodents, mammals, aquatic mammals, domestic animals such as dogs and cats, farm animals such as sheep, pigs, cows and horses. Wherein the terms “animal” or the plural “animals” are used, it is contemplated that it also applies to any animals.

As used herein the phrase “conservative substitution” refers to substitution of amino acids with other amino acids having similar properties (e.g., acidic, basic, positively or negatively charged, polar or non-polar). The following six groups each contain amino acids that are conservative substitutions for one another: 1) alanine (A), serine (S), threonine (T); 2) aspartic acid (D), glutamic acid (E); 3) asparagine (N), glutamine (Q); 4) arginine (R), lysine (K); 5) isoleucine (I), leucine (L), methionine (M), valine (V); and 6) phenylalanine (F), tyrosine (Y), tryptophan (W).

As used herein “conservative mutation”, refers to a substitution of a nucleotide for one which results in no alteration in the encoding for an amino acid, i.e., a change to a redundant sequence in the degenerate codons, or a substitution that results in a conservative substitution. An example of codon redundancy is seen in Tables 1 and 2.

TABLE 1
Amino Acids (Category-Based) and Triplet Code and
Redundant Corresponding Encoded Amino Acids
(Functional Group Category-Based)

| Nonpolar, | | | | Polar, | | | |
|---|---|---|---|---|---|---|---|
| aliphatic | | | | uncharged | | | |
| | Gly | G | GGT | | Ser | S | AGT |
| | | | GGC | | | | AGC |
| | | | GGA | | | | TCT |
| | | | GGG | | | | TCC |
| | | | | | | | TCA |
| | | | | | | | TCG |
| | Ala | A | GCT | | Thr | T | ACT |
| | | | GCC | | | | ACC |
| | | | GCA | | | | ACA |
| | | | GCG | | | | ACG |
| | Val | V | GTT | | Cys | C | TGT |
| | | | GTC | | | | TGC |
| | | | GTA | | | | |
| | | | GTG | | | | |
| | Leu | L | TTA | | Pro | P | CCT |
| | | | TTG | | | | CCC |
| | | | CTT | | | | CCA |
| | | | CTC | | | | CCG |
| | | | CTA | | | | |
| | | | CTG | | | | |
| | Met | M | ATG | | Asn | N | AAT |
| | | | | | | | AAC |
| | Ile | I | ATT | | Gln | Q | CAA |
| | | | ATC | | | | CAG |
| | | | ATA | | | | |
| Aromatic | | | | Positive | | | |
| | | | | charge | | | |
| | Phe | F | TTT | | Lys | K | AAA |
| | | | TTC | | | | AAG |
| | Tyr | Y | TAT | | His | H | CAT |
| | | | TAC | | | | CAC |
| | Trp | W | TGG | | Arg | R | CGT |
| | | | | | | | CGC |
| | | | | | | | CGA |
| | | | | | | | CGG |
| | | | | | | | AGA |
| | | | | | | | AGG |
| Negative | | | | OTHER | | | |
| charge | | | | | | | |
| | Asp | D | GAT | | stop | | TTA |
| | | | GAC | | | | TAG |
| | | | | | | | TGA |
| | Glu | E | GAA | | | | |
| | | | GAG | | | | |

TABLE 2
Redundant Triplet Code and Corresponding Encoded Amino Acids.

| | U | | C | | A | | G | |
|---|---|---|---|---|---|---|---|---|
| U | UUU | Phe | UCU | Ser | UAU | Tyr | UGU | Cys |
| | UUC | Phe | UCC | Ser | UAC | Tyr | UGC | Cys |
| | UUA | Leu | UCA | Ser | UAA | END | UGA | END |
| | UUG | Leu | UCG | Ser | UAG | END | UGG | Trp |
| C | CUU | Leu | CCU | Pro | CAU | His | CGU | Arg |
| | CUC | Leu | CCC | Pro | CAC | His | CGC | Arg |
| | CUA | Leu | CCA | Pro | CAA | Gln | CGA | Arg |
| | CUG | Leu | CCG | Pro | CAG | Gln | CGG | Arg |
| A | AUU | Ile | ACU | Thr | AAU | Asn | AGU | Ser |
| | AUC | Ile | ACC | Thr | AAC | Asn | AGC | Ser |
| | AUA | Ile | ACA | Thr | AAA | Lys | AGA | Arg |
| | AUG | Met | ACG | The | AAG | Lys | AGG | Arg |
| G | GUU | Val | GCU | Ala | GAU | Asp | GGU | Gly |
| | GUC | Val | GCC | Ala | GAC | Asp | GGC | Gly |
| | GUA | Val | GCA | Ala | GAA | Glu | GGA | Gly |
| | GUG | Val | GCG | Ala | GAG | Glu | GGG | Gly |

Thus, according to Table 2, conservative mutations to the codon UUA include UUG, CUU, CUC, CUA, and CUG.

As used herein, the term “homologous” means a nucleotide sequence possessing at least 80% sequence identity. preferably at least 90% sequence identity, more preferably at least 95% sequence identity, and even more preferably at least 98% sequence identity to the target sequence. Variations in the nucleotide sequence can be conservative mutations in the nucleotide sequence, i.e. mutations in the triplet code that encode for the same amino acid as seen in the Table 2.

As used herein, the term “therapeutically effective amount” refers to that amount of a therapy (e.g., a therapeutic agent or vector) sufficient to result in the amelioration of Angelman syndrome or other UBE3A-related disorder or one or more symptoms thereof, prevent advancement of Angelman syndrome or other UBE3A-related disorder, or cause regression of Angelman syndrome or other UBE3A-related disorder. In accordance with the present invention, a suitable single dose size is a dose that is capable of preventing or alleviating (reducing or eliminating) a symptom in a patient when administered one or more times over a suitable time period. One of skill in the art can readily determine appropriate single dose sizes for systemic administration based on the size of a mammal and the route of administration.

The dosing of compounds and compositions of the present invention to obtain a therapeutic or prophylactic effect is determined by the circumstances of the patient, as known in the art. The dosing of a patient herein may be accomplished through individual or unit doses of the compounds or compositions herein or by a combined or prepackaged or pre-formulated dose of a compounds or compositions. An average 40 g mouse has a brain weighing 0.416 g, and a 160 g mouse has a brain weighing 1.02 g, a 250 g mouse has a brain weighing 1.802 g. An average 400 g rat has a brain weighing 2 g. An average human brain weighs 1508 g, which can be used to direct the amount of therapeutic needed or useful to accomplish the treatment described herein.

Nonlimiting examples of dosages include, but are not limited to: 5.55×10¹¹ genomes/g brain mass, 5.75×10¹¹ genomes/g brain mass, 5.8×10¹¹ genomes/g brain mass, 5.9×10¹¹ genomes/g brain mass, 6.0×10¹¹ genomes/g brain mass, 6.1×10¹¹ genomes/g brain mass, 6.2×10¹¹ genomes/g brain mass, 6.3×10¹¹ genomes/g brain mass, 6.4×10¹¹ genomes/g brain mass, 6.5×10¹¹ genomes/g brain mass, 6.6×10¹¹ genomes/g brain mass, 6.7×10¹¹ genomes/g brain mass, 6.8×10¹¹ genomes/g brain mass, 6.9×10¹¹ genomes/g brain mass, 7.0×10¹¹ genomes/g brain mass, 7.1×10¹¹ genomes/g brain mass, 7.2×10¹¹ genomes/g brain mass, 7.3×10¹¹ genomes/g brain mass, 7.4×10¹¹ genomes/g brain mass, 7.5×10¹¹ genomes/g brain mass, 7.6×10¹¹ genomes/g brain mass, 7.7×10¹¹ genomes/g brain mass, 7.8×10¹¹ genomes/g brain mass, 7.9×10¹¹ genomes/g brain mass, 8.0×10¹¹ genomes/g brain mass, 8.1×10¹¹ genomes/g brain mass, 8.2×10¹¹ genomes/g brain mass, 8.3×10¹¹ genomes/g brain mass, 8.4×10¹¹ genomes/g brain mass, 8.5×10¹¹ genomes/g brain mass, 8.6×10¹¹ genomes/g brain mass, 8.7×10¹¹ genomes/g brain mass, 8.8×10¹¹ genomes/g brain mass, 8.9×10¹¹ genomes/g brain mass, 9.0×10¹¹ genomes/g brain mass, 9.1×10¹¹ genomes/g brain mass, 9.2×10¹¹ genomes/g brain mass, 9.3×10¹¹ genomes/g brain mass, 9.4×10¹¹ genomes/g brain mass, 9.5×10¹¹ genomes/g brain mass, 9.6×10¹¹ genomes/g brain mass, 9.7×10¹¹ genomes/g brain mass, 9.80×10¹¹ genomes/g brain mass, 1.0×10¹² genomes/g brain mass, 1.1×10¹² genomes/g brain mass, 1.2×10¹² genomes/g brain mass, 1.3×10¹² genomes/g brain mass, 1.4×10¹² genomes/g brain mass, 1.5×10¹² genomes/g brain mass, 1.6×10¹² genomes/g brain mass, 1.7×10¹² genomes/g brain mass, 1.8×10¹² genomes/g brain mass, 1.9×10¹² genomes/g brain mass, 2.0×10¹² genomes/g brain mass, 2.1×10¹² genomes/g brain mass, 2.2×10¹² genomes/g brain mass, 2.3×10¹² genomes/g brain mass, 2.40×10¹² genomes/g brain mass, 2.5×10¹² genomes/g brain mass, 2.6×10¹² genomes/g brain mass, 2.7×10¹² genomes/g brain mass, 2.75×10¹² genomes/g brain mass, 2.8×10¹² genomes/g brain mass, or 2.86×10¹² genomes/g brain mass. Other examples of dosages expressed as viral genomes per milliliter include, but are not limited to, 2.0×10¹³ vg/ml, 2.1×10¹³ vg/ml, 2.2×10¹³ vg/ml, 2.3×10¹³ vg/ml, 2.4×10¹³ vg/ml, 2.5×10¹³ vg/ml, 2.6×10¹³ vg/ml, 2.7×10¹³ vg/ml, 2.8×10¹³ vg/ml, 2.9×10¹³ vg/ml, 3.0×10¹³ vg/ml, 3.1×10¹³ vg/ml, 3.2×10¹³ vg/ml, 3.3×10¹³ vg/ml, 3.4×10¹³ vg/ml, 3.5×10¹³ vg/ml, 3.6×10¹³ vg/ml, 3.7×10¹³ vg/ml, 3.8×10¹³ vg/ml, 3.9×10¹³ vg/ml, or 4.0×10¹³ vg/ml.

The compositions used in the present invention may be administered individually, or in combination with or concurrently with one or more other therapeutics for neurodegenerative disorders, specifically UBE3A deficient disorders.

As used herein “patient” is used to describe an animal, preferably a human, to whom treatment is administered, including prophylactic treatment with the compositions of the present invention.

“Neurodegenerative disorder” or “neurodegenerative disease” or “neurological disorder” as used herein refers to any abnormal physical or mental behavior or experience where the death or dysfunction of neuronal cells is involved in the etiology of the disorder. Further, the term “neurodegenerative disease” as used herein describes “neurodegenerative diseases” which are associated with UBE3A deficiencies. Exemplary neurodegenerative diseases include Angelman's Syndrome, Huntington's disease, Alzheimer's disease, Parkinson's disease, amyotrophic lateral sclerosis, autistic spectrum disorders, epilepsy, multiple sclerosis, Prader-Willi syndrome, Fragile X syndrome, Rett syndrome and Pick's Disease.

“UBE3A deficiency” as used herein refers to a mutation or deletion in the UBE3A gene.

The term “normal” or “control” as used herein refers to a sample or cells or patient which are assessed as not having Angelman syndrome or any other neurodegenerative disease or any other UBE3A deficient neurological disorder.

Generally, a UBE3A vector was formed using a transcription initiation sequence, and a UBE construct disposed downstream of the transcription initiation sequence. The UBE construct is formed of a UBE3A sequence and a secretion sequence. Nonlimiting examples of the UBE3A sequence are SEQ ID No: 1, SEQ ID No: 2, SEQ ID No: 7, a cDNA of SEQ ID No:8, SEQ ID NO: 9, a cDNA of SEQ ID No: 10, or a homologous sequence. Variations of the DNA sequence include conservative mutations in the DNA triplet code, as seen in Tables 1 and 2. In specific variations, the UBE3A sequence is Rattus norvegicus UBE3A, Homo sapiens UBE3A variant 1, or Homo sapiens UBE3A variant 2.

Nonlimiting examples of the secretion sequence are SEQ ID No: 3, SEQ ID No: 5, SEQ ID No: 6, or a homologous sequence, with variations of the DNA sequence that include the aforementioned conservative mutations.

In some variations of the invention, the transcription initiation sequence is a cytomegalovirus chicken-beta actin hybrid promoter, or human ubiquitin c promoter. The invention optionally includes an enhancer sequence. A nonlimiting example of the enhancer sequence is a cytomegalovirus immediate-early enhancer sequence disposed upstream of the transcription initiation sequence. The vector optionally also includes a woodchuck hepatitis post-transcriptional regulatory element. The listed promotors, enhancer sequence and post-transcriptional regulatory element are well known in the art. (Garg S. et al., The hybrid cytomegalovirus enhancer/chicken beta-actin promotor along with woodchuck hepatitis virus posttranscriptional regulatory element enhances the protective efficacy of DNA vaccines, J. Immunol., Jul. 1, 2004; 173(1):550-558; Higashimoto, T. et al., The woodchuck hepatitis virus post-transcriptional regulatory element reduces readthrough transcription from retroviral vectors, September 2007; 14(17):1298-304; Cooper, A. R. et al., Rescue of splicing-mediated intron loss maximizes expression in lentiviral vectors containing the human ubiquitin C promoter, Nucleic Acids Res., January 2015; 43(1):682-90).

In variations, the vector is inserted into a plasmid, such as a recombinant adeno-associated virus serotype 2-based plasmid. In specific variations, the recombinant adeno-associated virus serotype 2-based plasmid lacks DNA integration elements. A nonlimiting example of the recombinant adeno-associated virus serotype 2-based plasmid is a pTR plasmid.

A method of synthesizing the UBE3A vector includes inserting a UBE3A construct into a backbone plasmid having a transcription initiation sequence. The UBE3A construct is formed of a UBE3A sequence and a secretion sequence as described above. For example, UBE3A gene variant 1 was cloned into a recombinant adeno-associated viral vector for expression of the secreted E6-AP protein in the brain and spinal cord of AS patients. A secretion sequence, such as GDNF, is added in frame on the 5′ end of hUBE3A. The UBE construct is optionally inserted by cleaving the backbone plasmid with at least one endonuclease, and the UBE3A construct ligated to the cleaved ends of the backbone plasmid.

The vector was then optionally inserted into an amplification host, possessing an antibiotic resistance gene, and subjected to an antibiotic selection corresponding to the antibiotic resistance gene. The amplification host was then expanded in a medium containing the antibiotic selection and the expanded amplification host collected. The vector was then isolated from the amplification host. In specific variations of the invention, the antibiotic resistance gene is an ampicillin resistance gene, with the corresponding antibiotic selection, ampicillin.

In an embodiment, a UBE3A vector is formed from cDNA cloned from a Homo sapiens UBE3A gene to form the UBE3A, variant 1 gene (SEQ ID No: 2) which is fused to a gene encoding a secretion signaling peptide, such as GDNF, insulin or IgK. In a preferred embodiment, GDNF is used. The construct is inserted into the hSUb vector, under a CMV chicken-beta actin hybrid promoter (preferred) or a human ubiquitin c promoter. Woodchuck hepatitis post-transcriptional regulatory element (WPRE) is present to increase expression levels.

The human UBE3A vector is then transformed into an amplification host such as E. coli using the heat shock method. The transformed E. coli were expanded in broth containing ampicillin to select for the vector and collect large amounts of vector. Therapeutically effective doses of vector can then the administered to a patient as a gene therapy for treating Angelman syndrome or another neurological disorder having UBE3A deficiency. The vector may be administered via injection into the hippocampus or ventricles, in some cases, bilaterally. Dosages of the therapeutic can range between about 5.55×10¹¹ to 2.86×10¹² genomes/g brain mass or between about 2.0×10¹³ vg/ml to 4.0×10¹³ vg/ml.

There are several animal models that recapitulate the phenotype of human patients such as motor dysfunction, inducible seizures, deficit in context dependent learning, electrophysiological impairment (long term potentiation (LTP). These animal models include maternal deficiency models (m−/p+) such as the mouse model (deletion of exon 2) and the rat model previously developed by the inventors (full gene KO). The rat model exhibits a loss of UBE3A protein, motor deficits, cognitive impairments, and electrophysiology deficits. (Dodge et al. 2020).

The following non-limiting examples illustrate exemplary compositions and methods of treatment thereof in accordance with various embodiments of the disclosure. The examples are merely illustrative and are not intended to limit the disclosure in any way.

### Example 1—UBE3A Protein is Present in Cerebrospinal Fluid (CSF) of Wild Type Rats but Absent in AS Rats

The inventors have found that catalytically active UBE3A protein is detectable within cerebrospinal fluid (CSF) of wild type rats but distinctly absent in AS rat CSF. Microdialysis within the rat hippocampus also showed that UBE3A protein is located in the interstitial fluid of wild type rat brains but absent in AS animals. This protein maintains catalytic activity and appears to be regulated in a dynamic activity-dependent manner.

With the generation of the novel AS rat model, the inventors were able to more easily analyze CSF for UBE3A protein due to the larger volumes that can be collected. The inventors were the first to identify UBE3A protein is present in wild type rat CSF and not in AS rat CSF. Alongside western blot analysis, mass spectrometry was used to confirm the presence of UBE3A protein in the CSF. These findings were replicated in wild type and AS mice. To determine if this deficit translated into human AS patients, five human neuro-typical samples were obtained for testing. Utilizing a hemoglobin ELISA, samples were examined for blood contamination to determine if the samples were suitable for further testing. Using human samples which showed no hemoglobin contamination, the presence of UBE3A protein was confirmed in the human samples. Obtaining human AS CSF samples is quite difficult due to the rarity of the disorder, although the inventors were very fortunate to receive three samples with the aid of the Foundation for Angelman Syndrome Therapeutics (FAST). The first sample, demonstrated a loss of UBE3A protein in the CSF by western analysis. Unfortunately, the other two AS samples had significant levels of hemoglobin and showed positive results on western analysis. This made it impossible to draw a definitive conclusion on the presence of UBE3A protein within human AS CSF. Although these results cannot confirm UBE3A protein within the AS human population, they do demonstrate the presence of UBE3A within neurotypical human CSF.

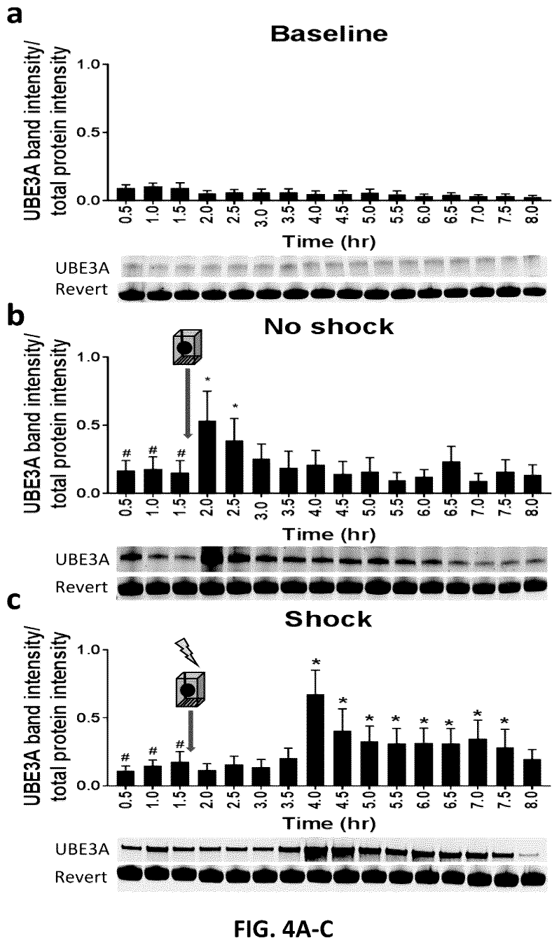

Determining the presence of UBE3A within the CSF suggested that UBE3A may have an as yet unknown extracellular function within the brain. Therefore, the inventors investigated whether UBE3A protein is present in the extracellular space within the rat brain. Hippocampal dependent learning and memory deficits are very prominent in AS making the hippocampus the region of interest. Microdialysis is a technique which allows for analysis of protein concentration changes within the interstitial fluid in an awake, free moving animal. This technique has many advantages with each animal serving as its own control from baseline levels being assessed which means the number of experimental animals needed is much smaller. Samples can be collected from the same animal from many different time points and it can be used in many different brain regions. WT and AS rats were utilized and dialysate was collected every 30 min over an 8 hour period. Samples run on a western blot confirmed the presence of UBE3A protein within the dialysate, which appeared stable over the 8 h of sampling (FIG. 4a).

To further implicate UBE3A in having a role in the extracellular space, the inventors sought to determine if UBE3A maintains its catalytic activity within the interstitial fluid, as well as the CSF. Ubiquitin ligase activity was observed for both the hippocampal dialysate as well as the CSF, and as expected ATP was required for the enzyme activity. It has been shown that ubiquitin, as well as circulating proteasomes, are located within the extracellular space in neuro-typical humans (Sixt & Dahlmann, 2008; Takada, et al., 1997; Wang, et al., 1991), suggesting that there may be other functioning E3 ligases present in the dialysate samples. To determine that the ubiquitination activity is due to the presence of UBE3A and not other E3 ligases, UBE3A immuno-depletion was used to demonstrate that loss of UBE3A protein resulted in ablation of ubiquitin ligase activity. As could be expected, no activity was seen from samples taken from AS rats. Since UBE3A expression is maintained in glial cells due to paternal gene expression within AS animals (although at much lower level than in neurons), it seems logical that lack of extracellular UBE3A in AS rats suggests that the UBE3A secretion is likely from neurons.

Activity-dependent regulation of UBE3A during learning paradigms could indicate one aspect of UBE3A's importance in learning and memory. Greer (2010) previously reported that within primary neuronal culture, following neuronal depolarization, there was a significant increase in both nuclear and cytoplasmic UBE3A levels (Greer, et al., 2010). Conversely, if neuronal activity was chemically blocked, there was a significant decrease in Ube3a mRNA (Greer, et al., 2010). Furthermore, it was demonstrated that UBE3A expression dramatically changes in a time-dependent manner following associative fear conditioned learning, with brain region specific profiles of expression (Filonova, et al., 2014). It was determined that both the maternal and paternal allele follow the same expression pattern within each brain region.

The inventors explored whether extracellular UBE3A protein undergoes a similar regulation by using the same associative fear conditioned learning in conjunction with microdialysis. As a control, animals were placed in the fear conditioning chamber without applying a shock. Interestingly, in the no shock control, the inventors noticed that immediately after exposure to the fear conditioning chamber, there was a significant increase in total UBE3A protein. This immediate increase in extracellular UBE3A rapidly declined within an hour. This was not seen in the group of animals which were maintained in the microdialysis chamber, which showed no change in baseline levels. The inventors associated this increase with the experience of being placed in a novel environment. A previous report demonstrated that exposure to a novel environment leads to a significant increase in UBE3A expression within the first hour of exposure (Greer, et al., 2010).

Interestingly in rats that were exposed to the full fear conditioning paradigm, there was a significant increase in UBE3A expression within the extracellular space at around two hours after the shock. This increase in UBE3A was maintained significantly higher than baseline levels for a longer duration than the increase seen in animals from the no shock group. This suggests that the more adverse learning experience had a different and significant effect on UBE3A protein release. The maintenance of higher levels for longer time may suggest that either an increase in release or reduction in UBE3A clearance from the extracellular space is occurring. UBE3A protein in the extracellular space may have a functional role in consolidation of more long term memory storage since the no shock animals had a limited increase in UBE3A protein. The absence of the immediate increase in UBE3A protein in the shock group, which was observed in the no shock group, is puzzling and certainly requires further investigation. This phenomenon was replicated with 8-9 individual animals in each group, suggesting an important and significant finding. UBE3A regulation may depend on the type of learning, which can be explored using different cognitive tasks in association with microdialysis. Since testing was conducted using only an aversive stimulus as the conditioning for memory formation, the possibility cannot be excluded that the alterations in extracellular UBE3A protein expression are due to the stress and pain, and not necessarily related to the memory formation itself. Thus, further studies of other learning paradigms that do not involve pain/stress would be of critical importance.

Ubiquitin and proteasomes have been reported being located in neuro-typical human serum, plasma, and CSF (Takada, et al., 1997; Wang, et al., 1991). Extracellular ubiquitin has been shown to play a role in many different pathways, such as being an endogenous agonist of the chemokine receptor CXCR4 which leads to inducing calcium ion influx into the cell. Extracellular ubiquitin can cause cell differentiation, as well as mediate cell growth and apoptosis (Sujashvili, 2016). The source of extracellular ubiquitin is currently unknown, albeit reports have determined one mode of secretion via T-lymphocytes inhibiting cytotoxic activity of platelets (Pancre, et al., 1991). The recent identification of ubiquitin and proteasomes having a role extracellularly, makes a case for UBE3A having a role outside of the cell. The only known mechanism of ubiquitination occurs from the ligase pathway implying that ligases are presumably located in the extracellular space. Reports have demonstrated that upon neuronal depolarization in primary neuronal cell culture that there is a decrease in UBE3A membrane localization (Filonova, et al., 2014), perhaps suggesting a release into the extracellular space. UBE3A has also been shown to be associated with endomembranes of the Golgi apparatus, presynaptic vesicles and terminals as well as postsynaptic density (Burette, et al., 2017; Burette, et al., 2018).

It is still unclear how UBE3A protein is secreted as UBE3A does not contain a putative secretion sequence. The association of UBE3A and the Golgi apparatus (Burette, et al., 2017; Burette, et al., 2018; Condon, Ho, Robinson, Hanus, & Ehlers, 2013) may indicate that UBE3A could go through the conventional secretory pathway. However, unconventional mechanisms of protein secretion have been reported, demonstrating that proteins do not have to go through the endoplasmic reticulum and Golgi apparatus to be secreted (Rabouille, Malhotra, & Nickel, 2012). One unconventional pathway involves recruitment of cytoplasmic proteins in vesicular compartments of the endocytic membrane system which fuse with the plasma membrane to release proteins into the extracellular space (Dimou & Nickel, 2018). Interestingly, during microdialysis with the aversive stimulus, the increase in UBE3A protein is delayed and maintained much longer than in the no shock paradigm. This could be due to newly synthesized UBE3A protein having to go through the secretory pathway and ensue transportation to be secreted. This is only speculation although could potentially be an explanation for the delay in extracellular level changes.

The inventors have shown the presence of an E3 ligase in the extracellular space. Demonstrating that extracellular UBE3A is under activity-dependent regulation leads to the idea that synaptic receptors could be a potential target. E3 ligases have tight regulation over learning and memory intracellular components and it is very likely there is a similar function in the extracellular space. For example, UBE3A has been shown to intracellularly target receptors that have major implications in learning and memory such as the SK2 receptor which has a direct effect of NMDA receptors (Khatri & Man, 2019; Sun, et al., 2015). Intracellular ubiquitination by E3 ligases other than UBE3A, have significant impacts on learning and memory, an example being Nedd4 whose role involves the internalization of AMPA receptors which are critical for synaptic plasticity (Lin, et al., 2011). To further understand the functioning of UBE3A outside of the cell, the inventors have conducted electrophysiological studies and application of exogenous UBE3A to hippocampal slices and identified that long-term potentiation deficits in AS slices can be modified with UBE3A protein administration as discussed in Example 2 below.

### RESULTS

### UBE3A is Present in the CSF

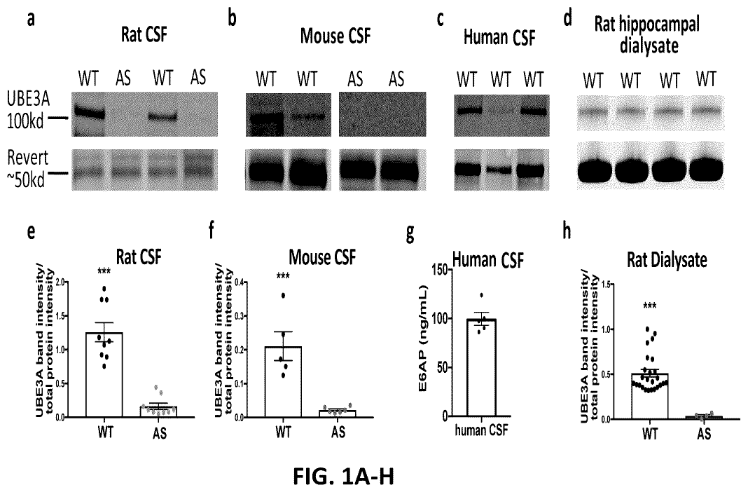

The inventors previously created a novel AS rat model (Dodge, et al., 2020), which has allowed for easier exploration of CSF for analysis due to the larger volumes that can be obtained. In efforts to find a potential biomarker, the inventors identified that wild type rats, but not AS rats, have UBE3A present in their CSF (FIG. 1a). This was further demonstrated in the AS mouse model (FIG. 1b). Western blot analysis of neuro-typical human samples (n=5) also demonstrated the presence of UBE3A in CSF (FIG. 1c). Western analysis using known amounts of recombinant UBE3A as a standard curve (2-80 ng), the amount of UBE3A protein in the human CSF is estimated to be ˜100±7 ng/mL (FIG. 1).

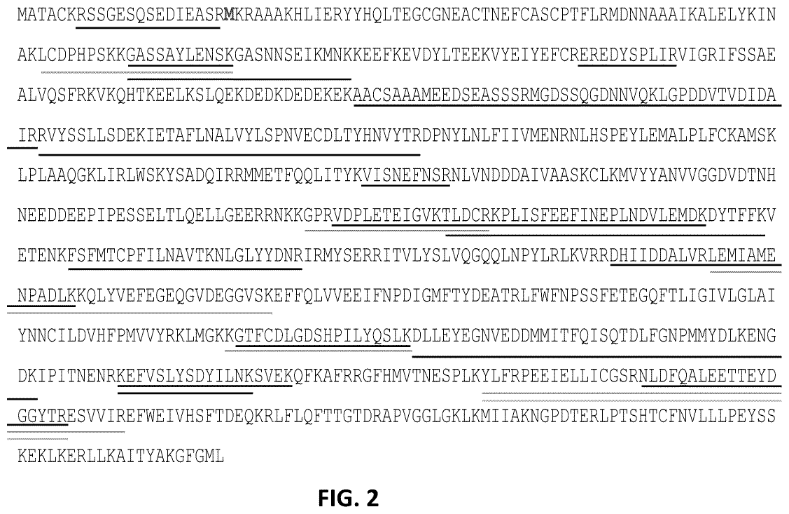

Further confirmation of the presence of UBE3A protein was achieved by using immunoprecipitation followed by mass spectrometry analysis. Through mass spectrometry the inventors identified 22 peptide fragments which belonged to rat UBE3A (aligning to 13 different regions), thus definitely confirming the identity of UBE3A by western analysis. Peptides identified are shown in FIG. 2. One peptide corresponds to a region on isoform 1 that is not present in isoform 3, suggesting that at least some of the protein present is isoform 1. Further work is required to determine if only one specific isoform is present.

It is exceedingly difficult to obtain human AS CSF samples, although 3 AS CSF samples have been obtained to date. The initial sample presented negative by western analysis, however, the latter two samples tested positive by western analysis (data not shown). This was likely due to contamination of blood in the CSF samples as indicated by significant levels of hemoglobin determined by ELISA (>800 ng/mL). This has made it difficult to conclusively confirm the results observed in the AS rat in the human condition. The neuro-typical human samples used for analysis showed no significant contamination of hemoglobin within the CSF.

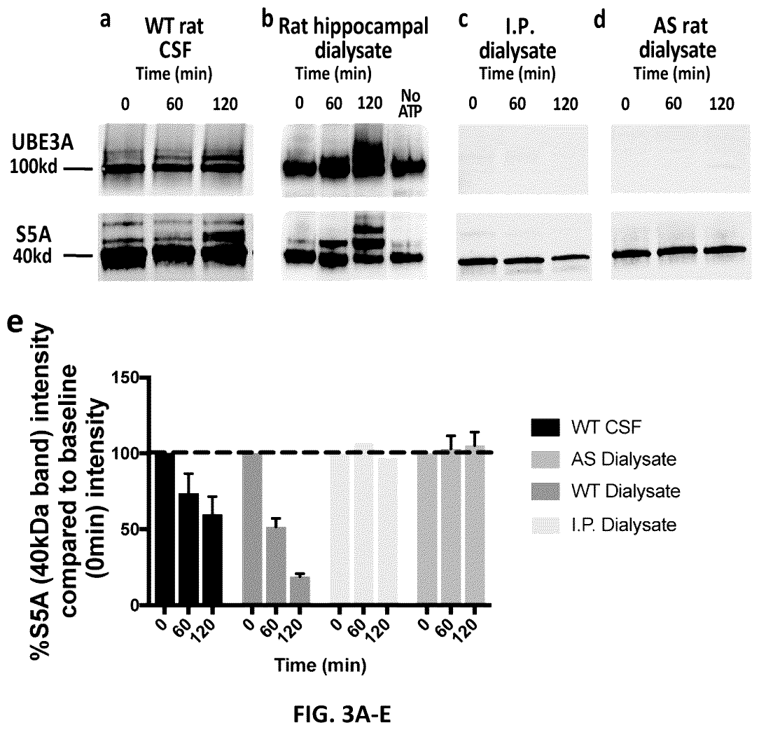

Discovery of UBE3A protein in the CSF lead to the question as to where this protein may originate. Since Hippocampal dependent learning and memory deficits are very prominent in AS, the inventors examined if UBE3A protein is located in the extracellular space of brain regions known to be important in learning and memory. Focusing on the hippocampus, microdialysis was utilized to sample interstitial fluid in awake, freely moving, WT rats. Samples were collected every 30 minutes for 8 h and western blotting was used to demonstrate the presence of UBE3A protein in the extracellular space of the hippocampus (FIG. 1d). Utilizing the AS rats, the inventors confirmed by microdialysis, that UBE3A protein was greatly reduced in the extracellular space (FIG. 3d).

### Activity of Extracellular UBE3A

The inventors sought to determine if UBE3A protein, within the extracellular space and CSF, maintains its catalytic activity for self-ubiquitination as well as ubiquitination of substrates. The inventors utilized a ubiquitination kit from Boston Biochem containing a well-known UBE3A substrate, S5A. Due to the difference in molecular weights of UBE3A (100 kDa) and S5a (50 kDa), the same blot was used to probe for self-ubiquitination, as well as s5a, without concerns of stripping the blot or antibody interactions. As subsequent 8 kDa ubiquitins are added to the proteins, the molecular weights of S5a and UBE3A gradually increase over time. FIG. 3a-b demonstrates that the UBE3A protein present in both the CSF and hippocampal dialysate were active in ubiquitinating both the S5A substrate and UBE3A.

Previous reports demonstrate the presence of ubiquitin, and proteasomes within neuro-typical interstitial fluid which could implicate other functioning E3 ligases within the extracellular space (Sujashvili, 2016)(Sixt & Dahlmann, 2008). To confirm the assay is showing an increase in ubiquitination from UBE3A protein, dialysate samples were immuno-depleted of UBE3A. Microdialysate samples were incubated with anti-UBE3A protein antibody and Sepharose G beads. The remaining immuno-depleted supernatant was ran through the assay. With UBE3A protein immuno-precipitated, UBE3A protein was not detected and the S5A band intensity remained unchanged over time, indicating that the previously observed ubiquitination was due to the presence of UBE3A activity (FIG. 3c). Similarly, assaying microdialysate from AS rats showed no UBE3A protein and no change in the S5A substrate band (FIG. 3d). As expected, removal of ATP from the assay abrogated ligase activity (FIG. 3b). These results demonstrate extracellular UBE3A protein within dialysate as well as in CSF, maintains its catalytic activity, undergoing self-ubiquitination, as well as ubiquitination of substrates.

### Extracellular UBE3A is Under Activity Dependent Regulation

It has previously been demonstrated in WT mice that Ube3a gene expression changes with learning and memory tasks, in particular fear conditioning (Filonova, Trotter, Banko, & Weeber, 2014). To determine if this increased UBE3A expression is associated with a concomitant increase in secretion of UBE3A protein during learning tasks, microdialysis in conjunction with fear conditioning was utilized. A control group was maintained in the microdialysis chamber with no exposure to the fear conditioning chamber. As a second control the inventors placed a group of animals in the fear conditioning chamber without applying the tone or shock, termed “no shock” group. Dialysate was collected every thirty minutes for eight hours following two hours of habituation to the microdialysis chamber. Animals that were subjected to the fear conditioning chamber were initially placed in the microdialysis chamber for 3.5 h with 1.5 h of baseline recordings. In control animals that were maintained in the microdialysis chamber only, there was no change in the basal level of secreted UBE3A protein over the 8 h of sampling (FIG. 4a). The inventors found that following exposure to the fear conditioning chamber in the no shock group, there is an immediate significant increase in UBE3A protein after exposure to the novel chamber (FIG. 4b). However, there was a rapid decline to baseline levels within 60 min. In the shock treatment group where the animals were subjected to the complete fear conditioning paradigm, there was a significant increase at 2.5 h time point following the fear conditioning paradigm which persisted for ˜3.5 hours (FIG. 4c). These data suggest that release of intracellular UBE3A protein is activity dependent. Thus, secreted UBE3A protein and its ubiquitination activity could have a functional role in learning and memory

### Materials and Methods

### Animals:

UBE3A maternal deletion AS rats, described previously (Dodge, et al., 2020). Heterozygous female rats were bred with wild type male rats to produce maternal-deficient AS offspring and age matched wild type litter mate controls. Animals were genotyped as described previously (Dodge, et al., 2020). UBE3A null mutation C57B1/6 AS mice, described previously (Jiang, et al., 1998). Animals were obtained through breeding of heterozygous female mice with WT males to produce maternal-deficient AS offspring and age matched wild type litter mate controls. Animals were housed in a standard 12-hour light/dark cycle and supplied with food and water ad libitum at the University of South Florida, and were housed in groups of two per cage. All procedures were conducted in compliance with the NIH Guidelines for the Care and Use of Laboratory Animals and approved by the Institutional Animal Care and Use Committee of USF (approval number A4100-01).

### Rat and Mouse CSF Collection:

Animals were anesthetized with an intraperitoneal injection of Somnasol (0.1 mL/450 g) and placed into a stereotaxic unit. Their head was immobilized with ear bars and fur was removed from neck, aseptic technique was used to clean the area. A 27×3/4″ gauge butterfly needle (Exel) was placed into the cisterna magnum and CSF was pulled through tubing with a 1 mL syringe (BD). Samples were snap frozen on dry ice and stored in −80° C. until use. Rats wild type n=9 (3M/6F), AS n=9 (5M/4F); mice wild type n=5 (2M/3F), AS n=6 (4M/2F). From rats 50-150 μL of CSF was obtained and for mice 10-20 μL of CSF was collected per animal. For rat samples the inventors performed a hemoglobin ELISA (Eagle Biosciences, RT021-KG1) as per manufacturer's instructions to determine if CSF samples were contaminated with blood. Samples with hemoglobin greater than 500 ng/mL were excluded from analysis (all wild type samples were <200 ng/mL). For western blots 20 μL of rat CSF and 10 μL of mouse CSF was loaded used for analysis. A rat hemoglobin ELISA (Eagle Biosci. Inc, Nashua N H) was used to determine that low levels of blood were present in the samples collected.

### Western Blotting:

CSF (or dialysate) as mixed with an equal amount of loading buffer (2× lamalie buffer, BME) and heated to 95° C. for five minutes. Samples were loaded into an 18 well SDS-PAGE gel (Bio-rad 4-15%). Immediately after transferring the gels onto nitrocellulose transfer membranes (Bio-Rad), blots were incubated in Revert total protein stain (Li-Cor) at room temperature for 5 min. Followed by 2×3 minute incubations in revert wash buffer (33.5 mL CH₃COOH, 150 mL CH₃OH, 316.5 mL ddH2O). The blot was imaged for total protein (Odyssey, 700 nm). After imaging, the blot went through 3×5 min washes in 1×TBST (1× Tris-buffered saline with 0.1% Tween 20 (Sigma-Aldrich)). Then blocked in 1×TBST solution with 5% non-fat dry milk (Lab Scientific) at room temperature (23±2° C.) for 2 hours. The blots were then incubated in primary antibodies, anti-UBE3A (Sigma (E8655) or Millipore (MABS1683), 1:2,000), anti-S5A (Boston Biochem (AF5540), 1:10,000) diluted in 5% non-fat dry milk mixed in Tris-buffered saline with 0.1% Tween 20 and left overnight at 4° C. After incubation blots went into three 10-minute washes in Tris-buffered saline with 0.1% Tween 20. The blots were then incubated with goat anti-rabbit (or anti-mouse) LICOR IRdye IgG (H+L) (800cw, Neta Scientific) at 1:5,000. Membranes then went into three ten minute washes, and then imaged (Odyssey, 800 nm). Protein band density was analyzed using Image Studio Software. Total protein (Revert Li-Cor) was used as the standard control when performing densitometry and data expressed as the ratio of UBE3A band intensity/total protein intensity. Boston Biochem recombinant UBE3A (E3-230) protein was used as a standard to approximate UBE3A protein amounts in samples. Recombinant UBE3A protein (2-80 ng) was loaded onto the same western blot as CSF (20-30 μL) and microdialysate and a standard curve was generated to estimate the amount of protein in each sample.

### Immunoprecipitation:

Sepharose-G beads (25 μL Abcam) were rinsed with 1×PBS and centrifuged (1,000 RPM, 4° C., 2 min). The rinse was repeated three times. Antibody (Sigma a-UBE3A 1:10) was added to rat CSF (25 μL) or hippocampal dialysate (15 μL) and incubated at 4° C. for 3 hours with rocking. Following incubation, Sepharose-G beads were added to the sample/antibody mixture and incubated at 4° C. for 3 h. For in vitro activity assays, the sample was centrifuged and the supernatant collected and used in the in vitro assay. For mass spectrometry analysis the samples were centrifuged and supernatant was removed. The beads were washed with 1×PBS. Beads were then submerged in 50 μl of 30 mM ammonium bicarbonate (pH 8) followed by denaturation at 95° C. for 5 min. After allowing to cool to room temperature (23±2° C.) beads were reduced and alkylated with 45 mM DTT (to a 1/10 solution and incubated at 60° C. for 30 min) and 110 mM IAA (1/10 solution and incubated at room temperature, in the dark for 20 minutes) respectively. 200 ng of trypsin was added to each sample and the final volume brought up to 100 μl with 30 mM ammonium bicarbonate and allowed to digest over night at 37° C. The following morning an additional 200 ng of trypsin was added and allowed to digest for an additional 2 h. Samples were then acidified with a TFA to a final concentration of 1%, put on ice for 15 min, then spun down at 10,000 rpm room temperature for 10 minutes, and the resulting supernatant was cleaned using a Millipore™ ziptip and samples used in mass spectrometry.

### Mass Spectrometry:

Samples were then reduced with a final concentration of 20 mM dithiothreitol, heated at 95° C. for 10 minutes, then cooled before alkylating cysteines with the addition of 40 mM iodoacetamide, final concentration. Samples were then incubated in the dark for 20 minutes at room temperature, followed by the removal of any undissolved matter by centrifugation at 17,000×g for 10 minutes. The clarified supernatant was transferred to a new tube followed by addition of 12% aqueous phosphoric acid at 1:10 for a final concentration of 1.2% phosphoric acid. Six times the volume of S-TRAP protein binding buffer consisting of 90% aqueous methanol, 100 mM Tris, pH 7.1 was then added to the acidified protein and mixed well. The S-Trap micro column (Protifi brand) was placed in a 1.7 mL tube in order to retain flow-through. The sample mixture was then added into the micro column 200 μl at a time, followed by centrifugation of the micro column at 4,000×g for 1 minute, removal of the flow-through, and repeating the process until the entire sample had passed through the S-Trap. Protein bound within the protein-trapping matrix of the spin column was washed with 150 μL S-Trap buffer; centrifugation and removal of the flow through was then repeated for a total of 3 rounds. The S-Trap was then moved to a clean 1.7 mL sample tube for proteolytic digestion where 20 μL of digestion buffer containing 30 mM ammonium bicarbonate with 1 μg. Trypsin/Lys-C protease (Promega) was added to the micro column. To ensure no air bubbles remained between the protease digestion solution and the protein trap, gel loading tips were used. The S-Trap micro column was then capped to limit evaporative loss without forming an air tight seal and incubated in a heat block for 37° C. overnight. After digestion, peptides were eluted first with 40 μL of 50 mM TEAB and centrifuges at 4000×g for 1 min. An additional 40 μl of 0.2% formic acid in LC-MS grade H₂O was added and then centrifuged at 4,000×g for 1 min. Finally, to recover hydrophobic peptides, a final elution of 35 μL of 50% acetonitrile containing 0.2% formic acid was added with a final centrifugation at 5000×g for 5 min. All eluates were collected in the same tube to prevent transfer loss. Eluted peptides were centrifuged under vacuum until dryness and then resuspended in 0.1% formic acid in H₂O. Samples were sonicated 10 minutes in a water bath and centrifuged 17,000×g for 30 min to fully pull down any insoluble particulate before transferring the clarified peptide supernatant into autosampler vials.

### Lc-Ms Analysis:

Peptides were characterized using a Thermo Q-exactive-HF mass spectrometer coupled to a Thermo Easy nLC 1200. Samples separated at 300 nl/min on an Acclaim PEPMAP 100 trap (75 μm, 25 cm, c18 3 μm, 100A) and an Acclaim PEPMAP 100 Column (75 μm, 25 cm, c18, 100A) using a 120 minute gradient with an initial starting condition of 2% B buffer (0.1% formic acid in 90% Acetonitrile) and 98% A buffer (0.1% formic acid in water). Buffer B was increased to 28% over 90 minutes, then up to 40% in an additional 10 minutes. High B (90%) was run for 15 minutes afterwards. The mass spectrometer was outfitted with a Thermo nanospray Flex source with the following parameters: Spray voltage: 2.24, Capillary temperature: 200dC, Funnel RF level=40. Parameters for data acquisition were as follows: for MS data the resolution was 60,000 with an AGC target of 3e6 and a max IT time of 50 ms, the range was set to 400-1600 m/z. MS/MS data was acquired with a resolution of 15,000, an AGC of 1e5, max IT of 50 ms, and the top 30 peaks were picked with an isolation window of 1.6 m/z with a dynamic execution of 25s. Resulting data was searched using Thermo Proteome Discoverer 2.2 software. A fully reviewed Mouse database was downloaded from Uniprot which was used in the Sequest HT search. A full trypsin digestion with a maximum of 2 missed cleavages was selected including a precursor mass tolerance of 10 ppm and a fragment mass tolerance of 0.02 Da. Modifications included oxidation, n-terminal acetylation, and carbamidomethylation. The resulting peptides were then filtered for high confidence and validated with a confidence threshold of 0.01 (Target FDR).

### Ubiquitination Assay:

E6AP Ubiquitin Ligase Kit—S5a Substrate, Boston Biochem (#K-230) was used to test activity and performed as per manufacturers' instructions. Tubes with 46 μL assay volume were created each containing 1× reaction buffer, 1× Mg²⁺-ATP, 1× E1 enzyme, 1× His-S5A substrate, 1× E2 enzyme (UBE2D3), 28 μL of dialysate (or CSF n=10), and 1× ubiquitin. The reaction begins as soon as the ubiquitin is added into the tubes. Assays were then incubated at 37° C. Initial testing of the assay used 9 separate time points over 9.0 h. Upon establishment of the assay, the number of time points was decreased to three time points (over 2 h). The activity was terminated by addition of SDS PAGE sample loading buffer with 1M dithiothreitol (DTT) followed by quickly freezing on dry ice. These samples were then heated to 95° C. for 5 min and run on a western blot (Sigma a-UBE3A 1:2,000, α-S5A 1:1,000 Boston Biochem K-230 kit).

### Human CSF Collection and Hemoglobin ELISA:

Neuro-typical human CSF samples were purchased from Innovative Research Inc. (Novi, MA) (male (41 yrs), and one pooled sample) and Discovery Life Sciences (Huntsville, AL) (female (26yrs) and male (31 and 34yrs)). AS human samples were obtained with the coordinated assistance of the Foundation for Angelman Syndrome Therapeutics (FAST). Hemoglobin levels were analyzed to determine red blood cell contamination using the hemoglobin ELISA kit from Abcam as described by the manufacturer (Cambridge, MA, USA). Samples were excluded if hemoglobin levels were above 200 ng/mL (Neurotypical n=5, AS n=3). Western analysis of human CSF (30 μL) was performed as for the rat CSF samples.

Microdialysis with Associative Fear Conditioning:

The procedure began with a survival stereotaxic surgery on 4-5 month old rats. Animals were given a subcutaneous injection of analgesic (Carpofen, 10 mg/kg) 30 min prior to surgery. The rats were anesthetized with Isoflurane (3% induction, 1.5-2.0% maintenance) and animals placed on a WPI stereotaxic frame. A guide cannula (Amuza) was inserted into their left hippocampus (5.6 mm post bregma, +5.0 lateral, and 3.0 ventral from dura). The rats were singly housed and allowed to recover for two days, to allow a decrease in inflammatory response and recovery of the blood-brain barrier. Following recovery, rats were placed in the universal microdialysis cage (BASi) allowing them to move freely during collection. A 4 mm probe (Amuza) was inserted within the guide cannula at the beginning of the day and allowed to equilibrate for two hours before collecting samples. This timing was chosen from previous studies reporting that this timing allows for stable baseline measurements, reformation of the blood-brain barrier, and responsiveness to cognitive load (McNay, Fries, & Gold, 2000)(McNay, et al., 2010). Sterile filtered artificial cerebral spinal fluid with 2% BSA (Sigma) was perfused through the probe at a rate of 1.5 μL/min. The first group of control animals (termed baseline) (n=7, 3M/4F) were placed in the microdialysis chamber and dialysate samples were collected for 8 hours. For the two test groups (shock and no shock), baseline microdialysis measurements were collected for 1.5 h, immediately prior to testing conditions. The animals selected for fear conditioning (shock group) (n=8, 5M/3F), were placed into a fear conditioning apparatus (Ugo Basile) and allowed to habituate for three minutes. A 95 dB tone was played for 30 sec with a foot shock (1 mA) being applied at the last two seconds of the tone. The animals were allowed to recover in the fear conditioning chamber for 90 sec and then placed back into the microdialysis universal cage for the remainder of the 6.5 hour collection time. For the no shock controls (n=9, 6M/3F), following the 1.5 h dialysate collection for baseline, animals were placed in the fear conditioning apparatus and allowed to explore for 3 min with no tone or shock applied. Following exposure to the chamber the animals were placed back into the microdialysis universal cage for the remainder of the 6.5 hour dialysate collection. Animals were euthanized at the end of sampling.

### Statistics

Data was assessed for outliers, by group, prior to analysis. All values exceeding a minimum criteria of 2 standard deviations from the group mean were removed from subsequent analysis. An alpha of 0.05 was used for all main effects. Results are presented as mean±SEM. For microdialysis comparing averaged baseline measurements to each time point, an independent t-test was used. Statistical analysis was performed using SPSS software.

### Conclusion

UBE3A appears to have diverse and multiple actions with in neurons, with critical functions within the cytoplasm and nucleus (Avagliano Trezza, et al., 2019; Khatri & Man, 2019). The inventors have demonstrated a potential novel role of UBE3A with regard to its presence in the extracellular space and its implications in learning and memory. It has been unclear how the absence of a neuronal “housekeeping” gene, such as UBE3A, can result in the extent and severity of neuronal dysfunction and disruption in memory formation. It is becoming increasingly clear that UBE3A is involved in many pathways with many different functions.

### Example 2—Exogenous UBE3A Protein Supplementation Recovers Deficits in AS Patients

As noted previously, the inventors recently generated a novel Angelman syndrome (AS) rat model with a complete Ube3a gene deletion, that recapitulates the loss of UBE3A protein and shows cognitive and EEG deficits. The predominate AS mouse model was created by an exon 2 null mutation of Ube3a on chromosome 7. The resulting phenotype revealed a disruption of spatial and associative memory formation as well as hippocampal synaptic disruption (Jiang et al., 1998). This AS mouse model is an instrumental tool for understanding altered molecular pathways leading to the severe AS cognitive deficits and evaluating potential therapeutics. Specifically the AS mouse model demonstrates severe deficits in memory formation associated with spatial learning (Morris water maze) and associative fear conditioning as well as impairments in Schaffer collateral long-term potentiation (LTP) and long-term depression (LTD) (Jiang et al., 1998; Pignatelli et al., 2014) Synaptic plasticity changes are mirrored by alterations in both pre- and post-synaptic pathways. However, the mouse models are hampered by notable challenges including, but not limited to, strain influences and phenotypic inconsistency (Born et al., 2017; Huang et al., 2013). Therefore, there has been interest in the generation of new models for AS which could more closely reflect the human AS phenotype.

The inventors recently created a new AS rat model (rUbe3a^{m-/p+) resulting from a full maternal Ube}3a gene deletion (Dodge et al., 2020). The rUbe3a^{m-/p+} rat model displays deficits in learning and memory in behavioral paradigms such as fear conditioning and touchscreen discrimination (Berg et al., 2020; Dodge et al., 2020). The rUbe3a^{m-/p+} rats also showed significantly increased cortical and hippocampal delta EEG power similar to that reported in the AS mouse model and humans AS patients (Born et al., 2021). Analysis of epileptiform activity in juvenile and adult rats showed increased time spent in epileptiform activity and increased duration for behavioral recovery from generalized seizures in AS compared to WT rats (Born et al., 2021).

As described in Example 1, the inventors discovered that UBE3A protein is located in the cerebrospinal fluid (CSF) of WT rats, mice, and neuro-typical humans, but is absent in AS animal models (Dodge et al., 2021). Furthermore, the inventors demonstrated that UBE3A protein is released into the extracellular space of the hippocampus where it maintains its catalytic activity and is controlled by dynamic activity-dependent regulation. This raises numerous possibilities for the mechanism of action for UBE3A; specifically if UBE3A protein has a function in the extracellular space or if it passively secreted for clearance purposes. The inventors hypothesize that extracellular UBE3A protein may play a role in synaptic function, LTP induction and hippocampal-dependent memory formation.

In this Example, the inventors describe the effects of supplementation of exogenous UBE3A protein to hippocampal slices and intrahippocampal injection of AS rats. The inventors found that the rUbe3a^{m-/p+} rat model demonstrates deficits in hippocampal input/output and long-term potentiation (LTP) compared to litter mate controls. Application of exogenous UBE3A protein to hippocampal slices recovers synaptic plasticity deficits observed in LTP. Furthermore, injection of recombinant UBE3A protein into the hippocampus of the AS rat can rescue the associative learning and memory deficits seen in the fear conditioning task. These data suggest that extracellular UBE3A protein may play a role in synaptic function, LTP induction and hippocampal-dependent memory formation.

Input/output curves are a reliable method for quantifying overall synaptic transmission following a single stimulation of varying intensity. This curve nicely represents presynaptic function from measurements of the fiber volley and can correlate to post synaptic activation through the measurement of the slope of the fEPSP. There are inconsistent reports utilizing the Ube3a exon 2 null mutation mouse model as to presence or extent of an input/output deficit (Ciarlone et al., 2016; Ciarlone et al., 2017; Egawa et al., 2012; Jiang et al., 1998; Judson et al., 2016; Moreira-de-Sa et al., 2020; Pignatelli et al., 2014; Rotaru et al., 2018), but all agree there are synaptic deficits in LTP.

Here the inventors show that the rUbe3a^{m-/p+} rats have a significant deficit in the fiber volley and fEPSPs with respect to WT rats. This result suggests that the rUbe3a^{m-/p+} rat CA3-CA1 functional connectivity is lower. This could possibly be due to the reduced number of functional synapses within the Schaffer collaterals or an overall deficit in molecular mechanisms controlling synaptic function. Numerous altered pathways, pertinent to normal cognitive functioning, have been reported in the AS mouse model (E1 Hokayem et al., 2018; Lopez et al., 2017; Sun et al., 2015). This leads the idea that the culmination of many dysfunctional pathways leads to such severe deficits in both pre- and post-synaptic mechanisms. This rUbe3a^{m-/p+} rat model may offer a better model for investigating functional connectivity demonstrating a more prominent deficit in input/output.

LTP is a measure of synaptic plasticity thought to mimic mechanisms underlying memory formation and consolidation (Sah et al., 2008). LTP is broken down into three phases with the early phase heavily relying on kinase activity and the later phases depending on protein synthesis and remodeling/strengthening of synapses. LTP deficits are a hallmark phenotype in the AS null mutation mouse model (Jiang et al., 1998). Here, the inventors demonstrate a significant LTP deficit in the rUbe3a^{m-/p+} rat model in both initiation and maintenance. There are many reports in the AS mouse giving insight into potential mechanisms that could be altered leading to the manifestation of the significant deficit shown in the rUbe3a^{m-/p+} rat (Khatri and Man, 2019; Lopez et al., 2018). A significant report demonstrated, in the AS mouse model, proper calcium/calmodulin-dependent protein kinase II (CaMKII) signaling is required for early-phase NMDA receptor-dependent LTP induction and maintenance. Sites of CaMKII autophosphorylation in the AS mouse are shown to be altered leading to altered CaMKII regulation manifesting as LTP deficits (Weeber et al., 2003). SK2 receptors are regulators of NMDA receptor function and have been reported to be direct targets of E6AP ubiquitination. Deficits in Ube3a leads to increased SK2 levels directly impacting NMDAR activation consequently impairing LTP (Sun et al., 2015). While additional dysfunctional learning and memory pathways have been reported in the AS mouse model, these few examples give insight into how the loss of Ube3a can lead to severely altered LTP.

Extracellular ubiquitin is a relatively new finding and is poorly understood. With the few studies that have been conducted, it is becoming clear that extracellular ubiquitin has major implications in normal functioning. Extracellular ubiquitin has been reported to play roles in modifying cell differentiation and apoptosis, moderating platelet cytotoxicity and most notably receptor internalization and induction of calcium influx (Sixt and Dahlmann, 2008; Sujashvili, 2016). As noted in Example 1 above, UBE3A protein is present within the extracellular space in both CSF and hippocampal interstitial fluid in the rat. Extracellular UBE3A protein maintained its catalytic activity towards both itself, as well as a well-known substrate, S5A. Furthermore, extracellular UBE3A protein was shown to be under activity-dependent regulation following fear conditioning. Interestingly in rats that were exposed to the fear conditioning paradigm, there was a significant and sustained increase in the release of UBE3A protein into the extracellular space. UBE3A protein in the extracellular space may have a functional role in consolidation of more long term memory storage, as animals that did not receive a shock had a limited increase in UBE3A protein (Dodge et al., 2021). To further investigate if UBE3A protein plays a role in learning and memory from the interstitial fluid, in this Example, the inventors applied exogenous UBE3A protein to hippocampal slices just prior to LTP induction and observed a significant improvement of the LTP deficits in the AS rats. More surprisingly, intra-hippocampal injection of UBE3A protein could rescue contextual fear conditioning deficits. This is a noteworthy finding demonstrating that extracellular UBE3A protein has the ability to correct synaptic plasticity deficits from the extracellular space, suggesting a novel function for UBE3A protein.

Interestingly, administration of UBE3A protein to WT hippocampal slices appeared to reduce the amplitude of the LTP. This may suggest a dose dependent effect for extracellular UBE3A protein. An examination of a dose response in AS hippocampal slices may inform more on this. Increased UBE3A protein is known to cause a related neurodevelopmental disorder Dup15q. Dup15q is caused by a duplication of a portion of 15q11.2-13.1 chromosome which results in clinical symptoms similar to those observed in AS but typically lack the severe ataxia seen in AS (DiStefano et al., 2020; LaSalle et al., 2015). This duplication results in a duplication of the UBE3A gene, however, the pathogenic role of increased UBE3A levels in Dup15q syndrome has not been definitively proven and other genes in this chromosomal region could be contributing. It has been demonstrated that increased gene dosage of Ube3a results in glutamatergic synaptic transmission suppression as a result of reduced presynaptic activity and postsynaptic action potential coupling (Smith et al., 2011). A decrease is observed in the presynaptic response in wild type slices incubated with the exogenous UBE3A protein. Further exploration of extracellular UBE3A protein in Dup15q mouse models may contribute to the understanding of UBE3A protein's involvement in LTP in both of these diseases.

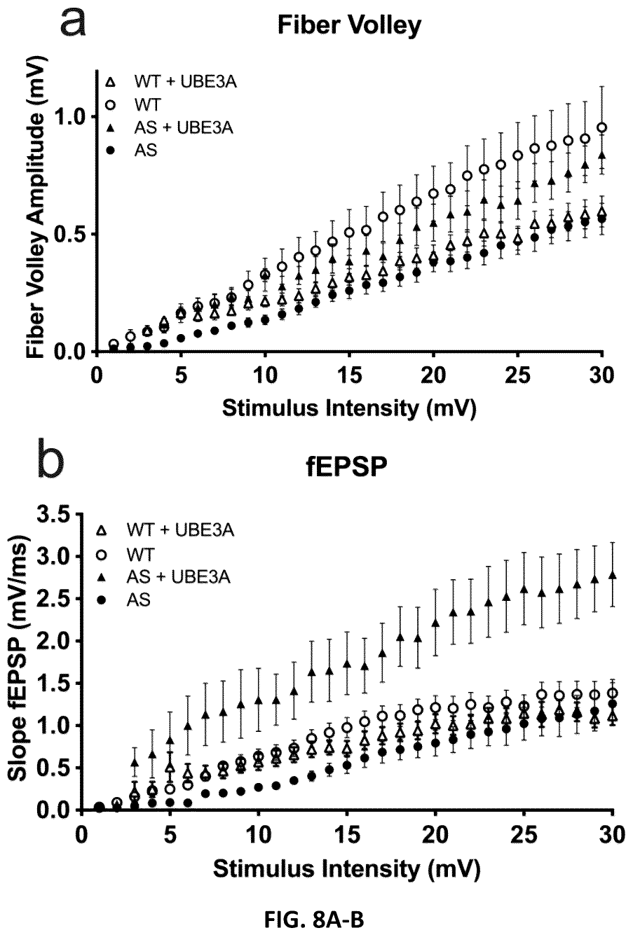

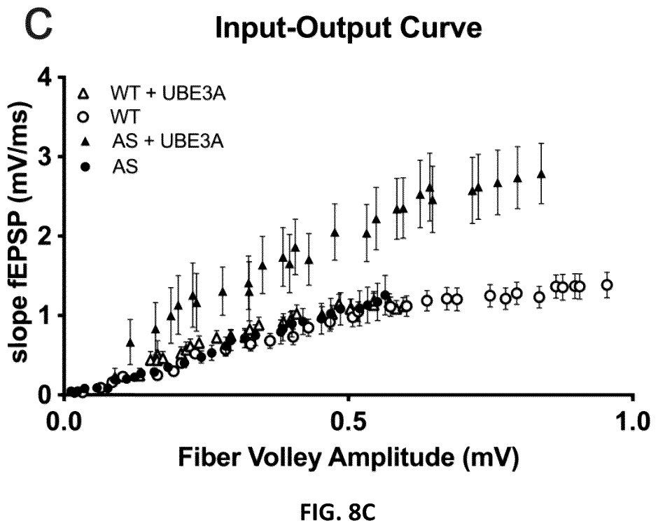

Demonstrating the recovery of LTP deficits through exogenous UBE3A protein application raises numerous questions. It is unclear how UBE3A protein is released into the extracellular space let alone how it is interacting with receptors or other extracellular proteins to modulate learning and memory. Given the previous observation of activity-dependent regulation of extracellular UBE3A protein (Dodge et al., 2021), UBE3A protein may be ubiquitinating synaptic receptors to alter their efficacy or activation. Of course one could speculate that exogenous administration of UBE3A protein to hippocampal slices could affect intracellular proteins if the protein is actively taken up, but it is more likely to affect proteins extracellularly in the short incubation time of 30 min. The data suggests that extracellular UBE3A protein is affecting both presynaptic and post synaptic responses in the rUbe3a^{m-/p+} hippocampal slices, with restoration of fiber volley and enhancement of fEPSP (FIG. 8).

### Results

### Rat Ube3a^{m-/p+} Deficits in Input/Output Curve

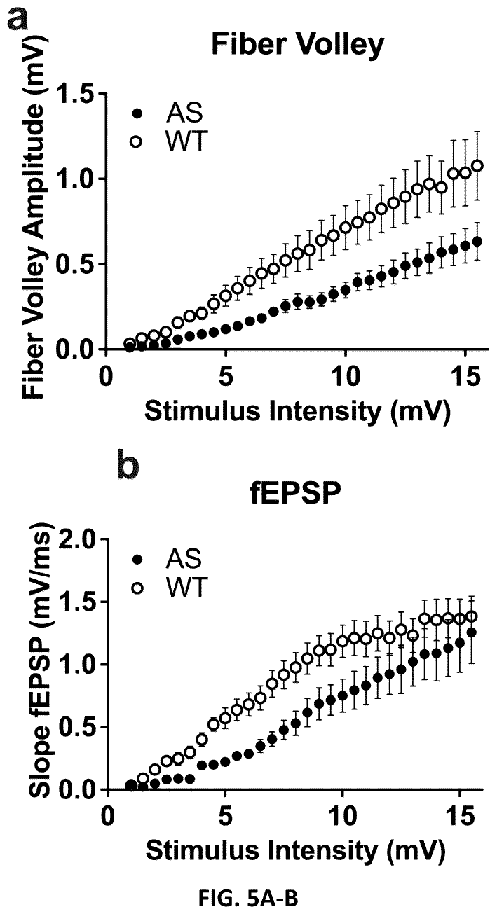

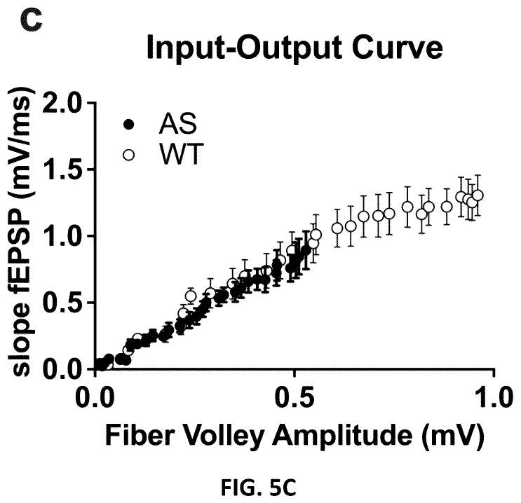

Input/output curves were determined prior to LTP induction and were measured from the slope of field excitatory post synaptic potentials (fEPSPs) elicited by stimuli of graded intensities from 0 to 15 mV at 0.5 mV increments. The fEPSPs are responses that arise as a manifestation of depolarization in the CA1 pyramidal neurons. The fiber volley is an indication of the pre-synaptic action potential arriving at the recording area. The stimulating electrode was placed in the CA3 Schaffer collaterals of the hippocampus while the recording electrode was placed in the CA1 stratum pyramidale. FIG. 5a shows the fiber volley with a significant reduction with increasing stimulus in the rUbe3a^{m-/p+} rats compared to WT littermates. FIG. 5b demonstrates an equivalent reduction in the fEPSPs with increasing stimulus. Collectively, there is a significant difference in the input/output curve (Figure Sc) in the hippocampus of the rUbe3a^{m-/p+} rats compared to the WT.

### Rat Ube3a^{m-/p+} Hippocampal LTP Deficits

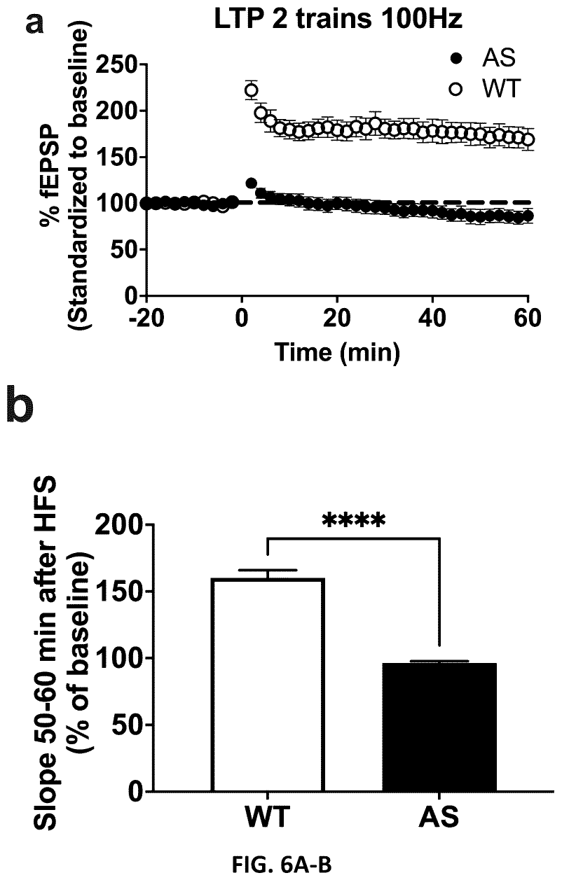

During input/output, the maximum response recorded was used to determine the stimulus intensity for the remainder of the experiment. Stimulus intensity was set to elicit approximately 50% of the maximum response recorded in input/output. This technique will allow the potential equivalent percentage of LTP despite the reduced rUbe3a^{m-/p+} rat input/output shown in FIG. 5. Baseline synaptic transmission was recorded for 20 min prior to tetanus to ensure a stable response. Following a stable baseline recording, the inventors next measured changes in fEPSP amplitude over time after high frequency stimulation (HFS, 2 trains of 100 Hz stimulation for 1 sec separated by 20 sec) to evaluate LTP at Schaffer collateral synapses. FIG. 6a demonstrates that the mean values of fEPSP slopes are significantly decreased in AS rats compared to WT littermates. FIG. 6b is an average of the last 10 min (50-60 min) after HFS demonstrating a significant deficit in the AS rat compared to WT littermates.

Extracellular UBE3A Protein Rescues rUbe3a^{m-/p+} LTP Deficits

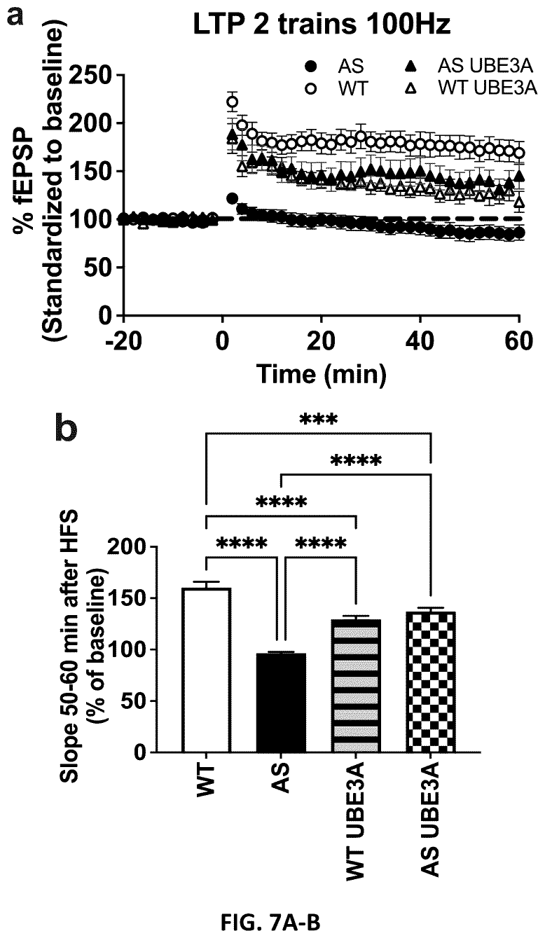

The inventors have previously demonstrated that UBE3A protein is present in the extracellular space and can be detected and quantified using microdialysis (Example 1). Furthermore, the release of UBE3A protein into the extracellular space was altered in a learning dependent manner, suggesting that extracellular UBE3A protein may play a role in synaptic plasticity and memory consolidation or is released in response to neuronal activation. Therefore, the inventors explored if supplementation of extracellular UBE3A protein to AS hippocampal slices could have beneficial effects and rescue of LTP deficits. UBE3A protein (80 nM; Boston Biochem (E3-230-050)) was incubated with hippocampal slices from rUbe3a^{m-/p+} rats for 30 min prior to initiating LTP recordings. It was found that preincubation with UBE3A protein was able to rescue rUbe3a^{m-/p+} slices to the same LTP response observed in control littermate rats (FIG. 7). This corresponded to a rescue of fiber volley indicating an improvement in presynaptic function (FIG. 8A). There also appears to be an enhanced post synaptic response (increased fEPSP) (FIG. 8B). This resulted in an increase in input/output (FIG. 8C). Interestingly, addition of UBE3A to WT hippocampal slices reduced the LTP by ˜20% (FIG. 7). This was associated with a decrease in presynaptic activity (decreased fiber volley, FIG. 8A), with no significant change in fEPSP (FIG. 8B). This resulted in a reduction in the input/out curve (FIG. 8C).

### Extracellular UBE3A Protein Rescues Fear Conditioned Learning and Memory Deficits

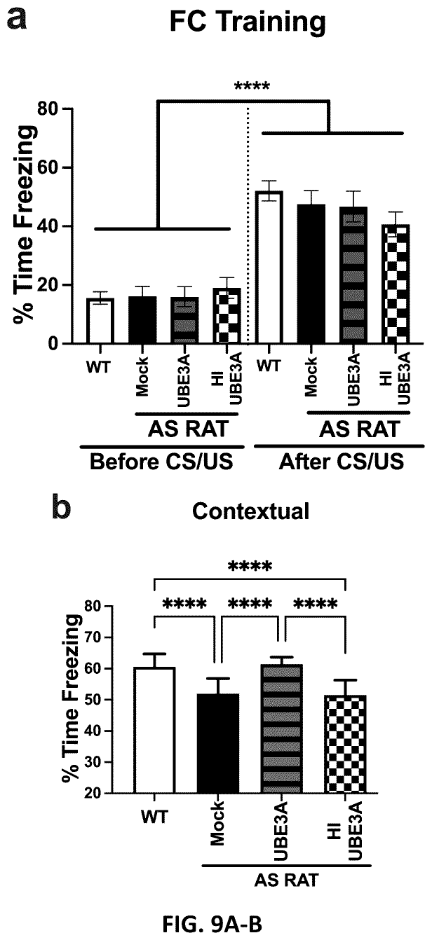

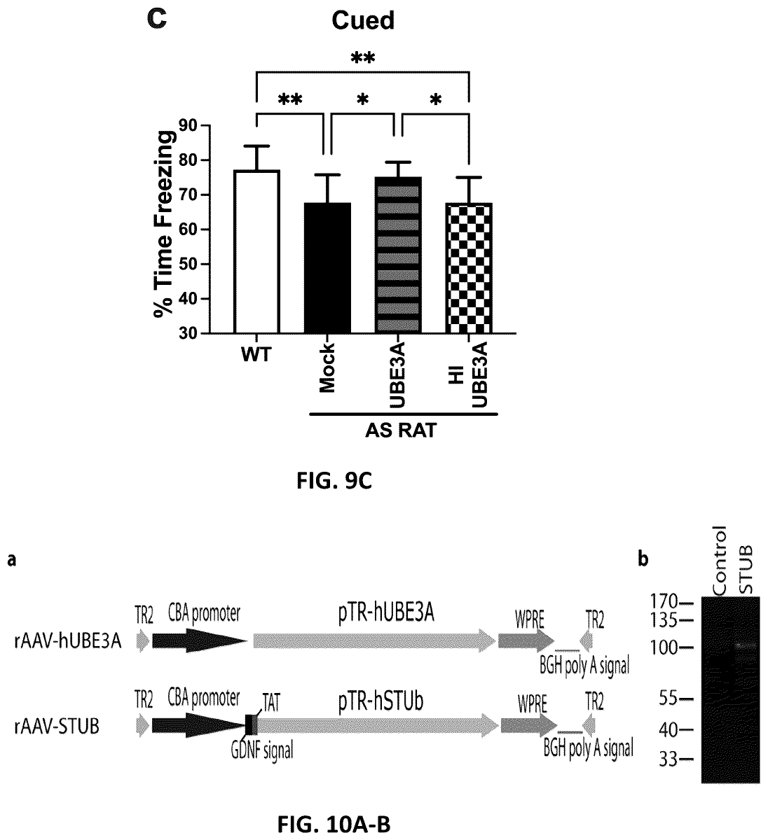

To assess if exogenous UBE3A protein can have an effect in vivo, rUbe3a^{m-/p+} rats were tested with hippocampal injections of UBE3A protein. rUbe3a^{m-/p+} rats were bilaterally injected with either PBS (Mock), UBE3A protein or heat inactivated UBE3A protein (HI-UBE3A) and allowed to recover overnight before training in fear conditioning, Heat inactivation was achieved by incubating UBE3A protein for 5 min at 95° C. A one foot-shock paradigm followed by a 72 h post-training contextual and cued test were utilized for fear condition. A control group of wild type rats was used as a reference. No significant differences were observed during fear conditioning training in any of the groups (FIG. 9A). However, as expected at 72 h post-training, the AS-Mock injected rats had deficits in both contextual and cued fear conditioning compared to WT animals (FIG. 9B&C), which was consistent with what the inventors have reported previously (Dodge et al., 2020). Surprisingly, AS-UBE3A protein injected animals showed a rescue in both contextual and cued fear conditioning compared to AS-Mock injected animals, with AS-UBE3A protein rats performing similar to WT rats (FIG. 9B&C). This effect was abrogated when the protein was heat inactivated prior to injections, suggesting that the enzymatic activity of UBE3A protein is required for this functional recovery.

### Materials and Methods

### Animals:

UBE3A maternal deletion AS rats (rUbe3a^{m-/p+}), described previously (Dodge et al., 2020). Animals were housed in a standard 12-hour light/dark cycle and supplied with food and water ad libitum at the University of South Florida, and were housed in groups of two per cage. All procedures were conducted in compliance with the NIH Guidelines for the Care and Use of Laboratory Animals and approved by the Institutional Animal Care and Use Committee of USF (approval number A4100-01).

### Extracellular Field Recordings:

Hippocampal slices were prepared from 4-5-month-old Ube3a maternal deficient rats and their wild-type littermates. Upon euthanasia by rapid decapitation, brains were rapidly removed and placed in ice-cold oxygenated (constant perfusion 95% O₂/5% CO₂) artificial cerebral spinal fluid (ACSF) containing (125 mM NaCl, 2.5 mM KCl, 26 mM NaHCO₃, 1.25 mM NaH₂PO₄, 25 mM glucose, 1 mM MgCl₂, 2 mM CaCl₂)). Brain slices (400 μm) were prepared on a vibratome (Thermo Scientific Microm HM 650V). Hippocampi were carefully dissected and allowed to equilibrate to room temperature (23±2° C.) in oxygenated ACSF for approximately 10 min. Slices were then transferred to nylon mesh recording chamber (temperature at 30.2° C., flow rate 1 mL/min) and allowed to equilibrate for a minimum of 2 hours. Stimulating electrodes (WPI, TM33CCINS) were placed in CA3 Schaffer collaterals and field excitatory post synaptic potential (fEPSPs) were recorded from the CA1 stratum radiatum via glass microelectrodes filled with ACSF (resistance 1-4 mΩ). Hippocampal area CA1 fEPSPs were recorded using Axon Digidata 1322A interface (Molecular Devices) data acquisition hardware operated by Axon pClamp 10.0 software. Signals were amplified with differential amplifier (A-M systems) filtered at 1 kHz and digitized at 10 kHz. Input/output was determined by stimulating slices from 0 to 15 mV at 0.5 mV increments (WT n=6 slices n=12, AS n=11 slices n=18). For all experiments, baseline stimulus intensity was set to elicit ˜50% of the maximum fEPSP response as determined from the input/output curve. LTP was induced by HFS which consisted of 2 trains of 100 Hz stimulation for 1 sec separated by 20 sec.

Extracellular field recordings data analysis: Data were analyzed using ClampFit 10.7 software, every 6 sweeps were averaged. Data were normalized to the averaged value of the initial slope of the fEPSP from the 20 min baseline recording. All data are represented mean±SEM (WT n=10 slices n=26, AS n=8 slices n=20). Independent t-test between genotypes (SPSS and GraphPad) were performed on the last 10 min average following induction method. Statistical significance was set at p<0.05.

### Intracranial Injections:

Immediately prior to surgery, rats were weighed and anesthetized with isoflurane. Surgery was performed using a World Precision Instruments stereotaxic apparatus. Nocita was used as a localized analgesia (50-100 μL at 13 mg/mL). The cranium was exposed using an incision through the skin along the midsagittal plane, and 2 holes were drilled through the cranium using a dental drill bit. Using a Hamilton microsyringe, injections of 3 μL of sterile phosphate buffered saline (PBS) or 3 μL of 80 nM recombinant human UBE3A protein (isoform 1) in PBS (Boston Biochem, E3-230) were dispensed bilaterally into the hippocampus (coordinates from bregma: lateral ±4.5 mm; anteroposterior −6.0 mm; vertical −5.0 mm) using the convection enhanced delivery method described previously (Carty et al., 2010). The concentration of 80 nM was estimated from a comparison of known concentrations of recombinant UBE3A protein to samples of rat hippocampal microdialysate using western blot analysis and anti-UBE3A antibody. The incision was cleaned and closed with surgical sutures. Animals were allowed to recover for 20-24 h before testing in fear conditioning. AS mock injected (n=11 (6F/5M)); AS UBE3A injected (n=12 (5F/7M) and wild type (n=14 (6F/8M) were 3-4 months of age. For heat inactivated human UBE3A protein, the protein was incubated at 95° C. for 5 mins.

### Fear Conditioning:

Fear conditioning testing was performed on the rUbe3a^{m-/p+} rats as described previously (Dodge et al., 2020). Briefly, the first day of training consisted of the rats exploring a 25 cm square sound attenuation chamber with a wire grid floor (Stoelting) for 3 mins prior to the presentation of a 1,000 Hz, 95 dB tone for 30 s and a mild foot shock (1 mA) during the last 2 s of the tone. Freezing was recorded as a measure of fear and was designated as a lack of movement for 1 consecutive sec by Ethovision XT soft-ware (Noldus). The second phase consisted of contextual and cued conditioning, which took place 72 h post-training. AS mock injected (n=13 (7F/6M)); AS UBE3A protein injected (n=12 (5F/7M)), AS HI-UBE3A protein injected (n=13 (6F/7M)) and wild type (n=20 (9F/11M)). Data was assessed for outliers, by group, prior to analysis using Grubbs outlier test (Alpha=0.05). One AS-Mock injected animal was identified and removed from analysis. A one-way ANOVA followed by a Tukey's post hoc test was used to analyze training, cued fear conditioning and contextual fear conditioning. An alpha of 0.05 was used for all main effects. Results are presented as mean±SEM. Statistical analysis was performed using GraphPad Prism software.

### Conclusion

The rUbe3a^{m-/p+} rat is a promising model that will complement the existing mouse model for the study of AS, as well as potential therapeutic interventions. The LTP deficits observed are consistent with the AS mouse model and presumably contribute significantly to the deficits in cognition that were observed previously (Berg et al., 2020; Dodge et al., 2020). Extracellular UBE3A protein expands the understanding of the role of UBE3A protein on LTP. UBE3A protein has previously been identified in both the cytoplasm and nucleus, with different isoforms showing different distributions (Burette et al., 2017; Burette et al., 2018; Sirois et al., 2020; Zampeta et al., 2020). UBE3A protein may have different functional activities within different cellular compartments within neurons. Understanding of the molecular mechanism that extracellular UBE3A protein has in LTP and cognition can lead to new approaches for therapeutic treatment for AS

### Example 3—Secreted Human UBE3A Improves Recovery of Behavioral and Electrophysical Deficits in AS Patients

For treatment purposes AS is considered a monogenic disorder, which is evidenced by AS patients with disease causing point mutations in UBE3A. This suggests gene replacement therapy as a promising avenue of treatment for this monogenic disorder. Investigations into gene therapy-based treatments for neurological disorders have been increasing for several years, especially for treatment of monogenic disorders. The recent FDA approval of gene therapy approaches for RPE65 mutation-associated retinal dystrophy and type I spinal muscular atrophy supports the progression for other monogenic disorders like AS. This makes UBE3A a clear target for a disease-modifying treatment. The inventors have previously shown recovery of deficits in the mouse model of AS using recombinant adeno-associated viral vectors (rAAV) expressing mouse Ube3a [Daily et al. 2011]. While there is a clear potential for rAAV-mediated gene therapy, the primary challenge is getting rAAV to transduce a majority of UBE3A-deficient neurons, while minimizing high titer and the number of injections. The inventors examined rAAV vector diffusion and transduction limitations in order to develop effective rAAV-based central nervous system (CNS) therapies and developed a novel secreted form of UBE3A which was combined with intracerebroventricular (ICV) injections in a rat model of AS.

As noted previously, the inventors recently developed a new AS rat model which was generated using CRISPR technology resulting in a full deletion of the rat Ube3a gene. This rat model displays the typical loss of Ube3a expression within neurons, disruption in general motor coordination, gait alterations, increased cortical and hippocampal delta EEG power, as well as sociability and memory deficits [Born et al. 2021; Berg et al. 2020; Dodge et al. 2020]. These studies were the first to identify the presence of extracellular UBE3A protein in the rat. While the protein is present in the brain of wild type (WT) animals, extracellular UBE3A in the AS rat was absent [Dodge et al. 2021]. The levels of extracellular UBE3A appeared to be regulated in an activity-dependent manner, with increases in UBE3A in the fear conditioning task [Dodge et al. 2021]. These data strongly suggested that UBE3A may have an important role extracellularly for learning and memory. As such, release of UBE3A protein from transduced cells may be advantageous in a gene therapy approach.

In light of the foregoing, the inventors explored a combination of two approaches to facilitate the distribution of UBE3A throughout the CNS. ICV injections, which can give increased vector distribution, were combined with a novel secreted human UBE3A construct created by the inventors and termed STUB (Secreted TAT UBE3A). STUB was designed with a secretion signal along with a cell penetrating peptide sequence. This allows the vector to create cellular “protein factories” from transduced cells in which the protein will be created and secreted. The cell-penetrating peptide sequence allows neighboring non-transduced cells to take up the protein. In addition to demonstrating effective use of the human UBE3A gene in a rAAV as an AS treatment, the data also show that STUB, via ICV injection, has a greater effect compared to the native non-secreted UBE3A vector in rescuing behavioral and electrophysiological deficits in the Ube3a deletion rat model of AS [Dodge et al. 2020].

### Results

### Generation of the STUB Construct

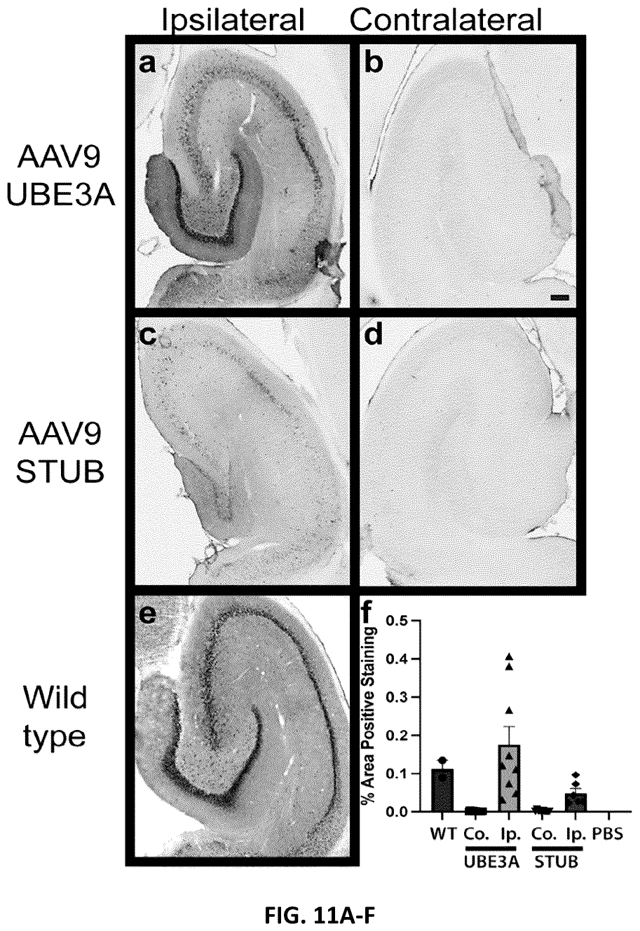

To optimize the efficiency of rAAV-based gene therapy for the CNS, the inventors designed a secreted form of hUBE3A termed STUB (Secreted TAT UBE3A) (FIG. 10a). This construct was created by cloning a secretion signal and a cell penetrating peptide sequence onto the N-terminus of hUBE3A into an rAAV plasmid. The STUB construct was first tested for expression in mammalian cells via cell culture using HEK293 cells and compared to unmodified human UBE3A. Due to low sensitivity and nonspecific bands when using an anti-UBE3A antibody with the culture media, an HA tag was added to the C-terminus of each construct for this experiment only. The STUB construct with HA was designated as STUB-HA. Secretion of STUB-HA into the cell culture media (with no detection seen by the unmodified UBE3A-HA) was observed as expected, indicating that the secretion signal peptide was functional (FIG. 10b). Expression of STUB protein generated by rAAV was validated using the AS rat model and IHC staining (FIG. 11). UBE3A staining was found in the injected hemisphere of the HPC but not the contralateral hemisphere. The AAV-UBE3A staining appeared stronger than the AAV-STUB (FIGS. 11a & c) even though the titer for the UBE3A was slightly lower. The most likely explanation is due to secretion. From the inventors' experience with other proteins that have been secreted, what appears to be lower expression is observed but is likely just due to a decrease in staining because of a more diffuse presence of the protein in the extracellular space. [Carty et al. 2013]

### Functional Activity of STUB

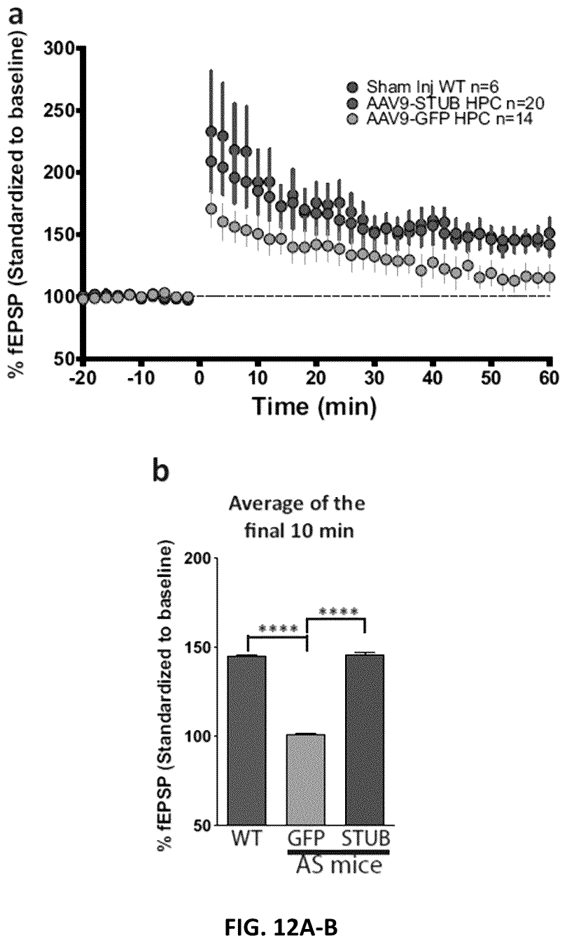

After confirmation of vector expression, the inventors wanted to ensure that STUB would generate active protein after the addition of the modifications. First, STUB was tested for its ability to recover the hippocampal LTP deficit present in AS mice. AS mice were injected bilaterally into the hippocampus with either rAAV-STUB or a rAAV-GFP control vector and hippocampal electrophysiology was performed as described previously [Daily 2011]. A complete recovery of the hippocampal LTP deficit in the AS mice injected with rAAV STUB was observed compared to control injected animals (FIG. 12). This rescue was similar to that observed previously with the mouse Ube3a gene in AAV [Daily 2011]. Further, it demonstrates that the modifications to hUBE3A to generate STUB did not alter its functional ability to rescue this deficit in animal models.

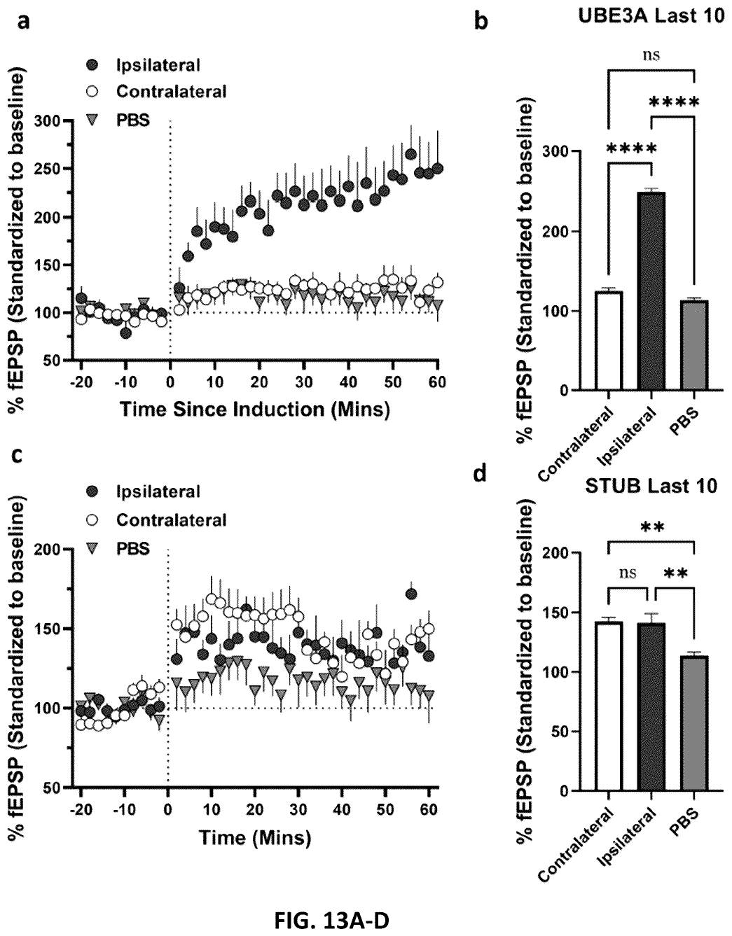

The inventors next wanted to confirm potential secretion and further characterize functional activity of STUB in vivo. Given that hippocampal structures are connected across hemispheres by the hippocampal commissural fibers [Shinohara et al. 2012], delivery of STUB to the ipsilateral hippocampus could have potential benefits on the contralateral side due to secretion of STUB. This effect should not be observed with gene delivery of the native hUBE3A because limited release of protein into the contralateral hippocampus would be expected. Ube3a^{m-/p+} rats were injected unilaterally into the hippocampus with either PBS, rAAV-hUBE3A or rAAV-STUB. Rats recovered for at least 8 weeks to allow for gene expression and then LTP was induced in both the ipsilateral and contralateral hippocampus. The AS rats showed a significant LTP deficit and that expression of either hUBE3A or STUB was able to recover this deficit in the ipsilateral hippocampus (FIG. 13). More importantly, recovery of LTP deficits in the contralateral hippocampus for rAAV-STUB injected animals was shown, whereas non-secreted hUBE3A injections failed to recover deficits in the contralateral hippocampus (FIG. 13). These data suggest that the STUB construct produced a functional UBE3A protein, and based on the ability of rAAV vectors to take advantage of axonal transportation this protein was likely being secreted from cells in the injected hippocampus into the contralateral hippocampus. [Shinohara et al. 2012; Wang et al. 2021; Green et al. 2016; Castle et al., Molecular Therapy 2014; Castle et al., Human Gene Therapy 2014]

### Effects of ICV Gene Replacement Therapy Injections on Hippocampal LTP

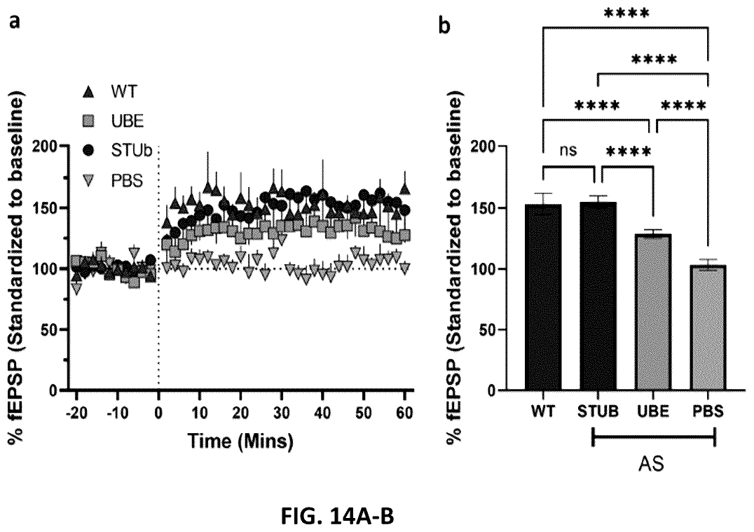

In examining the potential recovery of the LTP deficits in AS rats using ICV injections of rAAV-STUB and rAAV-hUBE3A, the inventors observed a significant improvement in hippocampal LTP in the rAAV-hUBE3A injected group compared to the control injected AS animals (FIG. 14). This result showed that the human UBE3A gene can recover LTP in the AS rat in a similar manner to what was observed previously using rAAV with the mouse Ube3a gene in the AS mouse [Daily 2011], although it did not completely achieve the level of potentiation of wild type animals (FIG. 14). Interestingly, the administration of rAAV-STUB was able to recover LTP to a level indistinguishable from WT animals (FIG. 14), suggesting that the STUB construct is more effective for LTP recovery when delivered ICV than the hUBE3A vector.

Behavioral Testing of Animals Treated with ICV STUB

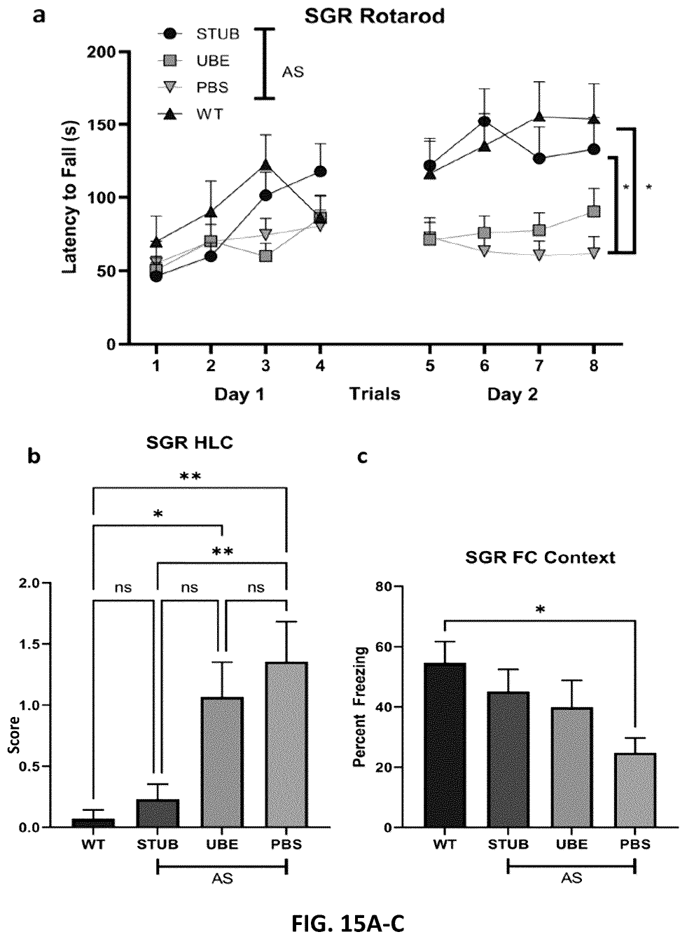

To determine if the effects seen in the recovery of electrophysiological deficits translated to a functional recovery, behavioral tests were performed on Ube3a^{m-/p+} rats ICV injected with rAAV-STUB, rAAV-hUBE3A, or PBS. After 8 weeks to allow for gene expression, animals were tested on the accelerating rotating cylinder (rotarod) test to determine effects on locomotor abilities. By the end of the second day of testing, STUB treated animals showed a latency to fall comparable to WT levels, whereas hUBE3A treated animals were indistinguishable from the PBS treated control group (FIG. 15a). Animals were also examined for hind limb clasping. Although there was a trend for a recovery, the rAAV-hUBE3A group was not significantly different from the PBS group (FIG. 15b). On the other hand, the rAAV-STUB group showed significant improvement and a recovery to WT levels. Both tasks suggest an improvement in motor function with the rAAV-STUB treatment but not with the rAAV-hUBE3A treatment. To examine the effects of ICV treatment on associative learning and memory deficit in the Ube3a^{m-/p+} animals, fear conditioning testing was performed. Results showed a partial recovery in the freezing behavior of the STUB and UBE3A groups above the PBS control group but not completely to wildtype levels (FIG. 15c), suggesting an improvement in learning and memory in the treated animals.

The inventors have demonstrated an improved AS gene therapy using two rodent models of AS through their innovative secreted UBE3A construct delivered via rAAV. This novel secreted form of human UBE3A variant 1 showed expression both in cell culture and in vivo before functional testing was performed. After adding modifications allowing for the secretion and reuptake of the protein, the inventors first sought to verify that these changes did not affect its treatment potential in the AS mouse. The initial functional examination in vivo showed that the STUB construct did not lose the ability to recover the LTP deficit in the AS mouse model, indicating that it was suitable for further study. More importantly, when a unilateral hippocampal injection of STUB was performed, LTP was improved in both the ipsilateral and contralateral hemispheres. This supports the notion that STUB was able to secrete to the contralateral hemisphere and is thus acting in a manner consistent with its design. The inventors do not believe that the improvement seen on the contralateral side is due to anterograde or retrograde transport of the rAAV, which has previously been reported, because the non-secreted hUBE3A failed to show improvements in the contralateral hippocampus. [Wang et al, 2021; Green et al. 2016; Castle et al., Molecular Therapy 2014; Castle et al., Human Gene Therapy 2014]

A second interesting aspect is the level of expression on the contralateral side. By immunohistochemistry, there was little increase in the levels of detectable UBE3A protein in the contralateral hippocampus, which might suggest that a detectable increase in the level of UBE3A is not needed to achieve a therapeutic effect. This may suggest that a low level of transduction could be sufficient to have significant effects in vivo. Further studies exploring the levels of secreted hUBE3A protein using microdialysis could help determine the minimally required levels of UBE3A protein needed for therapeutic improvements. It is important to point out that the recovery seen could be due to an undetermined mechanism such as simply increasing overall synaptic function which results in an increase in LTP.

One major roadblock of translating gene therapy from animal models into the patient, is addressing the significantly larger volume of the human brain alongside restrictions in the amount of vector that can be introduced to patients. In addition to the engineering of STUB, the inventors increased distribution through ICV injections. The brain's ability to transport molecules through cerebrospinal fluid throughout the brain makes targeting the ventricles a good strategy for gene replacement therapy. Several groups have had success in using ICV injections to enhance rAAV-based treatments. [Janson et al. 2014; Wolf et al. 2011; Belur et al. 2021]. CSF administration of rAAV vectors is also currently being explored in clinical trials for other neurological disorders (e.g., Study NCT03634007). The inventors show a similar increase in distribution using ICV injections with the use of rAAV-STUB and rAAV-hUBE3A. Both STUB and hUBE3A vectors showed significant improvements in hippocampal LTP suggesting that this delivery method could be advantageous for a therapeutic delivery route. The STUB vector also showed additional improvements over the hUBE3A vector, with improved recovery of behavioral deficits in the Ube3a^{m-/p+} rat model. This rescue suggests that secretion of hUBE3A adds an additional enhancement to the vector design above native hUBE3A.

One potential explanation for the improved function of STUB is an increase in extracellular UBE3A protein levels. The inventors' recent discovery of extracellular UBE3A protein suggests the possibility of an important extracellular function of UBE3A protein and suggested that release of UBE3A protein from cells could have potential effects on neighboring cells. [Dodge et al. 2021] Assuming this is the case, it could be unnecessary to transduce every cell to recover UBE3A function in the CNS. In fact, the results show that by treating one hemisphere with STUB, recovery of hippocampal LTP is found both ipsilaterally and contralaterally. The inventors offer two mutually non-exclusive explanations for the results demonstrating STUB as superior to non-secreted UBE3A in ICV injections: 1) STUB increases spread of the UBE3A protein and with the TAT sequence allows uptake by neighboring cells resulting in an increase in the number of cells effected by the therapy; and 2) STUB increases the extracellular pool of UBE3A which is important for improved synaptic function. It is likely that UBE3A has important functions in many compartments of the neurons. However, if UBE3A has a critical extracellular function, then STUB has a greater potential to increase the levels of extracellular UBE3A protein. Additionally, this would also benefit neighboring non-transduced cells. Further studies exploring a purely secreted UBE3A may help reveal if it is primarily one phenomenon over the other or a synergetic effect of the two.

It is important to emphasize that adult rats were used in this study. The animals were injected at 3 months of age, an age representing young adulthood [Sengupta 2013]. Previous work with an Cre-dependent inducible Ube3a AS mouse suggested that there are distinct developmental windows in which Ube3a restoration can restore AS deficits [Silva-Santos et al. 2015]. In that study they reinstated Ube3a at 4 distinct ages, embryonic, juvenile (3 weeks), adolescent (6 weeks) and adult (14 weeks) and then tested the animals at 10, 16, 22 and 28 weeks of age, respectively. LTP was rescued at all ages. Motor deficits were rescued in embryonic and juvenile treated animals with some improvement in adolescent animals but no improvements in adult animals. Interestingly, anxiety, repetitive behavior, and seizures were only rescued when Ube3a was reinstated during early development thus suggesting that there may be a time window for which an effective therapy could be given to patients, and that adult AS patients may have limited recovery of most aspects associated with this disease. This may explain the improvement in learning and memory but not complete recovery in fear conditioning task in this study. Earlier intervention with these vectors could give a much greater rescue of the AS phenotype and should be explored in future studies.

The inventors do not see rescue of the motor deficits in the adult AS rat with the injections of rAAV-hUBE3A, a finding that is consistent with the conditional mouse study as well as the previous rAAV delivery of mouse Ube3a. However, surprisingly a rescue of the motor deficits in adult rats with the injection of rAAV-STUB was shown. This is a highly significant finding as no other therapies have shown motor rescue in adult animals with the exception of the recent exciting work by Adhikari et al. (2021) demonstrating a rescue using a lentiviral transduced stem cell transplant [Adhikari et al. 2021]. Whether this motor rescue in the STUB but not the UBE3A injected animals is due to a significant improvement in the number of cells receiving the therapeutic or due to an increase in the extracellular UBE3A remains to be seen. Understanding the molecular mechanism for this rescue would certainly advance the understanding of UBE3A biology and function in neurons.

### Materials and Methods

### Animals

Ube3a deletion rats were bred and maintained as described previously [Dodge et al. 2020]. 129-Ube3a^{tm1Alb}/J mice [Jiang et al. 1998] were bred and maintained as described previously [Daily et al. 2011]. AS mice were used for initial characterization of the electrophysiological testing before moving to the rat model for more in depth testing. Male and female animals were housed in a standard 12-hr light/dark cycle and supplied with food and water ad libitum at the University of South Florida and were group-housed. The investigators were blind to treatments for all behavior testing. All procedures were conducted in compliance with the NIH Guidelines for the Care and Use of Laboratory Animals and approved by the Institutional Animal Care and Use Committee of USF (approval number A4100-01).

### Construct Generation and Vector Production

The human UBE3A gene variant 1 (GenBank: BC002582.2) (SEQ ID NO: 2) was cloned into the rAAV expression vector pTR12.1-MCSW. This vector has AAV2 terminal repeats, CBA/CAG promoter, WPRE and BGH poly signal (FIG. 10a). Starting with the hUBE3A construct, a secretion signal—(ATGAAGTTATGGGATGTCGTGGCTGTCTGCCTGGTGCTGCTCCACACCGCG TCCGCC) (SEQ ID NO: 3) and a cell-penetrating peptide sequence—(TAT, TACGGCAGAAAGAAGAGGAGGCAGAGAAGGAGA) (SEQ ID NO:4) were added in frame on the 5′ end of hUBE3A (FIG. 10a). For the in vitro experiment shown in FIG. 10b an HA tag was added to the vector in order to easily detect the protein. For the remaining work, the HA-tag free vector was used. In vitro testing was performed by transfecting HEK293 cells with the STUB construct using lipofectamine and incubating for 72 h before media was collected for further testing via western blot. Viral particles were generated as previously described [Daily et al. 2011; Burger et al. 2016]. Briefly, HEK293 cells were co-transfected with either the pTR-hUBE3A plasmid or the pTR-hSTUB plasmid, pXX6 helper plasmid and pAAV9 serotype plasmid using polyethyleneimine transfection. Virus was harvested from cells and purified using an iodixanol gradient and concentrated using centrifugal filtration. Viral titer was determined with a dot blot assay as described previously [Burger et a; 2016]. Vectors are described as vector genomes (vg)/mL.

### Western Blotting:

Samples were heated at 95° C. for 5 minutes before running on an SDS-PAGE gel with Laemmli sample buffer containing β-mercaptoethanol. Protein was transferred to a nitrocellulose membrane using the Trans-Blot Turbo system (Bio-Rad) and REVERT stain (Li-Cor Biosciences) was used to quantify total protein before blocking with Intercept T20 blocking buffer (Li-Cor Biosciences). Blots were incubated overnight at 4° C. with gentle rocking. For UBE3A detection the inventors used anti-UBE3A primary antibody solution (Sigma-Aldrich, SAB1404508) at 1:1000 dilution. For anti-HA detection the inventors used the Roche antibody clone 3F10 at 1:1000 dilution. Blots were incubated in secondary antibody (Li-Cor Biosciences, IgG-800CW) at 1:10,000 for 2 hr at room temperature. Detection was performed using the Odyssey CLx scanner (Li-Cor Biosciences). Analysis was performed using LI-COR Image Studio. Data are shown as the ratio of UBE3A band intensity/total protein intensity (REVERT).

### Immunohistochemical (IHC):

To test for expression of the newly generated STUB construct in vivo IHC staining was performed. Animals were anesthetized with a lethal dose of ketamine/xylazine then perfused with PBS before removal of the brain and submersion of the brain in cold 4% PFA. Tissue was fixed overnight before being incubated in 30% sucrose for 3 days. Sections were obtained with a microtome. Tissue sections were processed using the free floating IHC procedure with nickel and diaminobenzidine. Endogenous peroxidases were quenched with 0.3% hydrogen peroxide for 30 min. Blocking solution (5% horse serum, 0.25% Triton-X100 in PBS) was applied for one hour. Tissue sections were then incubated overnight with anti-UBE3A antibody at a 1:3000 dilution (Sigma-Aldrich, SAB1404508). After washing with PBS, secondary antibody (horse anti-mouse IgG(H+L), peroxidase, Vector Labs) was applied for one hour at room temperature. Color was developed using diaminobenzidine and nickel chloride with hydrogen peroxide. Tissue was mounted on glass slides before dehydration and cover slipping using DPX. Slides were scanned with the Zeiss Axioscan and analyzed using NearCyte. Data is shown as percent area positive DAB staining of the hippocampus normalized to the AS control group. One way ANOVA used to analyze data followed by Tukey's post-hoc test.

### Animal Injection:

Animals were anesthetized using isoflurane and positioned in a World Precision Instruments stereotactic surgery apparatus. An incision was made on the sagittal surface of the skull. AS mice were injected into the hippocampus (HPC) (X±2.7, Y-2.7, Z-3.0 from bregma) with 2 mL of either rAAV9-GFP (n=4: M=1, F=3) or rAAV9-STUB (n=7: M=3, F=4) at 1.0×10¹³ vg/ml. Wild type littermate controls were injected with 2 mL per site with sterile PBS (n=3M). For rats, the coordinates (X=±L 5, Y=−0.5, Z=−4.3 from bregma) were used for bilateral ICV injections and (X=4.6, Y=−6.0, Z=−5.0 from bregma) for unilateral intrahippocampal (IHPC) injections into the right hippocampus. A 10 mL volume for ICV and 2 mL volume for IHPC injections (per injection site) were applied at the coordinates above using convection enhanced delivery method [Nash et al. 2016; Carty et al. 2013]. Vectors were injected into AS rats at the following concentrations: hippocampal STUB, 3.4×10¹³ vg/ml (n=8: M=4, F=4); hippocampal UBE3A, 2×10¹³ (SD+/−0.56×10¹³) vg/ml (n=8: M=4, F=4); ICV STUB, 3.0×10¹³ (SD+/−0.58×10¹³) vg/ml (n=17:M=12, F=5); ICV UBE3A, 2×10¹³ vg/ml (n=15: M=10, F=5).

### Motor Deficit Testing:

Motor deficits were tested in rats using the hind limb clasping test (AS STUB n=13:M=10, F=3; AS UBE3A n=15: M=10, F=5; WT=11: M=6, F=5; AS PBS n=14, M=9, F=5) along with the accelerating rotarod (AS STUB n=17:M=12, F=5; AS UBE3A n=15: M=10, F=5; WT=14; M=8, F=6; AS PBS n=14, M=9, F=5). Testing was performed as previously reported [Dodge et al. 2020]. Hind limb clasping was performed by suspending the animal by the tail for 30 seconds and observing the hind limbs. The animal behavior was recorded and later scored by a researcher blind to animal treatment. The clasping phenotype was measured according to the commonly used 0-3 scale with 0 corresponding with no clasping, 1 with one withdrawn limb, 2 with both limbs withdrawn, and 3 with clasping together of the hind paws. Rotarod was performed by placing the animals onto an accelerating rotating cylinder (4-40 rpm, Ugo Basile) for up to 5 minutes and the latency to fall recorded. Testing was performed for 4 trials each day over two consecutive days with 30 minutes between each trial.

### Testing of Associative Learning:

Associative learning was tested using contextual fear conditioning as previously reported [Dodge et al. 2020] (AS STUB n=11:M=8, F=3; AS UBE3A n=12: M=8, F=4; WT=13: M=8, F=5; AS PBS n=13, M=8, F=5). For conditioning, animals were placed in a sound attenuation chamber and allowed to acclimate for 2 minutes. The conditioned stimulus tone (95 dB, 5000 Hz) played for 30 seconds with the animals receiving a 1 mA foot shock during the last two seconds. Animals then remained in the chamber for 3 minutes while freezing behavior (lack of movement for two consecutive seconds) was recorded. For the contextual testing, animals were placed back into the chamber 72 h after conditioning and freezing behavior was recorded. Freezing behavior was analyzed through tracking software (Ethovision XT).

### Electrophysiological Testing:

Mouse hippocampal long-term potentiation (LTP) was performed as described previously [Daily et al. 2011] (AS STUB injected n=20 slices, 7 animals; AS GFP injected n=14 slices, 3 animals; WT n=6 slices, 3 animals). Rat hippocampal LTP testing was performed on both IHPC and ICV cohorts (IHPC: AS STUB injected hemisphere n=5 slices, 3 animals: M=2, F=1; AS STUB uninjected hemisphere n=6 slices, 3 animals: M=2, F=1; AS UBE3A injected hemisphere n=5 slices, 3 animals: M=2, F=1; AS UBE3A uninjected hemisphere n=7 slices, 4 animals: M=2, F=2; AS PBS n=7 slices 4 animals: M=2, F=2. ICV: AS STUB n=11 slices, 5 animals: M=3, F=2; AS UBE3A n=11 slices, 5 animals: M=4, F=1; WT n=8 slices, 4 animals: M=2, F=2; AS PBS n=11 slices 4 animals: M=3, F=2). LTP was tested as previously reported with minor modifications [Weeber et al. 2003]. Briefly, the brain was quickly removed and immediately submerged in 95:5 02: CO2 saturated ice cold cutting solution. Sections were obtained using a vibrating microtome. Hippocampi were dissected and allowed to rest in a solution of 50:50 cutting solution: artificial cerebrospinal fluid (ACSF). Slices were transferred to the recording chamber and allowed to recover for 1 h 30 m. Stimulation was applied to Schaffer collaterals using a concentric bipolar electrode (WPI) and extracellular field recordings were obtained from neurons of the CA1. To induce LTP, theta burst stimulation, consisting of five trains of four pulses at 100 Hz with an inter-burst interval of 20 seconds, was applied at an intensity that resulted in ˜50% maximal response as obtained from an input/output curve. Data were analyzed using ClampFit 10.7 software. For analysis, six sweeps over two minutes were averaged, and data were normalized to the averaged value of the initial slope of the fEPSP from the 20-minute baseline recording. Data are presented as two minute average timepoints. All data are represented mean±SEM.

### Statistical Analysis:

Graphs were created using GraphPad Prism with data presented as mean with standard error of the mean. Comparison between two groups was done by t-test. For multiple groups, data was analyzed by one-way ANOVA followed by Tukey's multiple comparison test or mixed-effects ANOVA and Fisher's LSD. P value of 0.05 was chosen as the cutoff for significance. Significance was represented as follows: *p<0.05, **p<0.01, ***p<0.001, and ****p<0.0001.

### Conclusion

The work presented here in the AS rat (summarized in Table 3) builds upon the inventors' previous work in the AS mouse using a gene replacement approach and the recent observations of extracellular UBE3A [Daily et al. 2011; Dodge et al. 2021]. Gene replacement therapy with ICV injection of hUBE3A variant 1 resulted in no recovery of motor deficits, partial recovery of the cognitive deficit, and partial recovery of the LTP deficit. This suggests that ICV injection with WT hUBE3A may not be as effective as a direct hippocampal injection on cognitive function, however this may be due to a dosing issue. In contrast, protein replacement therapy with ICV injection of secreted hUBE3A variant 1 resulted in full recover of motor deficits, possible recovery of cognitive deficit, and full recovery of LTP deficit. This suggests that ICV injection with secreted hUBE3A (STUb) is an improved therapeutic intervention for AS.

Through the addition of secretion and cell penetrating peptides, this novel STUB construct can increase the effect of treatment beyond locally transduced neurons by supplying replacement of UBE3A to cells that were not transduced with the viral administration. Behavioral rescue with STUB to greater extent than standard AAV-mediated gene replacement raises an interesting premise for the treatment of specific learning and memory phenotypes observed in the Ube3a deficiency rat model. Either the greater distribution offered with STUB or the presence of extracellular UBE3A protein is responsible for this observation. The rescue of contralateral LTP following STUB treatment would suggest the latter explanation is the more likely.

TABLE 3
Comparison of AAV9-UBE3A and AAV9-STUB
phenotypic rescue in the AS rat

| Test/outcome measure | | UBE3A | STUB |
|---|---|---|---|
| HPC injection | Ipsilateral HPC LTP | Improved | Improved |
| | Contralateral HPC LTP | No improvement | Improved |
| ICV injection, | | Partial | Rescue |
| HPC LTP | | rescue | |
| Rotarod | | No rescue | Rescue |
| Hindlimb clasp | | No rescue | Rescue |
| Contextual fear | | Partial | Partial |
| conditioning | | rescue | rescue |

### Example 4—hSUB vector

The inventors have identified a functional role for extracellular E6AP in the previous examples. Briefly, dysregulation of synaptic plasticity underlies a large number of neurodegenerative disorders. Long-term synaptic plasticity is considered the neural basis of learning and memory process and LTP as well as LTD are the major forms of enduring synaptic strength changes of the central nervous system (Malenka 2004). The AS rat has deficits in both LTP and LTD. FIG. 5a shows the fiber volley with a significant reduction with increasing stimulus in the rUbe3a^{m-/p+} rats compared to WT littermates. FIG. 5b demonstrates an equivalent reduction in the fEPSPs with increasing stimulus. Collectively, there is a significant difference in the input/output curve (FIG. 5c) in the hippocampus of the r Ube3a^{m-/p+} rats compared to the WT. This translates into a reduction in LTP in the AS rat, with the mean values of fEPSP slopes showing a significant decrease compared to WT littermates (FIG. 6a). FIG. 6b is an average of the last 10 min (50-60 min) after high frequency stimulation (HFS) demonstrating a significant deficit in the AS rat compared to WT littermates.

The inventors also determined that the AS rat has a significant deficit in LTD (FIG. 17). Following a stable baseline measurement, slices were bathed in 10 μM of (S)-3,5-DHPG for 10 min. Following a 10 min washout of the DHPG, fEPSPs recordings were continued for 40 min. WT controls show expected synaptic depression stabilizing at approximately 20 minutes post DHPG application. In contrast, there is no detectable depression in AS rat slices for the duration of the experiment indicating a significant DHPG-induced LTD deficit in the AS rats compared to the WT rats (FIG. 17).

As shown in Example 1, the presence of extracellular E6AP was regulated in an activity dependent manner. In Example 2, the inventors determined whether supplementation of extracellular E6AP to AS hippocampal slices could have beneficial effects and rescue of LTP deficits. E6AP protein (80 nM) was incubated with hippocampal slices from rUbe3a^{m-/p+} rats for 30 min prior to initiating LTP recordings. Preincubation with E6AP protein was able to rescue rUbe3a^{m-/p+} slices (FIG. 7). This corresponded to a rescue of fiber volley indicating an improvement in presynaptic function (FIG. 8A). There also appears to be an enhanced post synaptic response (increased fEPSP) (FIG. 8B). This resulted in an increase in input/output (FIG. 8C). Interestingly, addition of E6AP to WT hippocampal slices reduced the LTP by ˜20% (FIG. 7). This was associated with a decrease in presynaptic activity (decreased fiber volley, FIG. 8A), with no significant change in fEPSP (FIG. 8B). This resulted in a reduction in the input/out curve (FIG. 8C). This suggests that there may be a concentration dependent effect, with high concentrations of E6AP having negative effects on LTP. This may indicate a possible mechanism for cognitive deficits seen in Dup15 syndrome and suggest that care may be required in AS therapies that try to increase the amount of E6AP expression.

To assess if exogenous E6AP can have an effect in vivo the inventors tested rUbe3a^{m-/p+} rats with hippocampal injections of E6AP. rUbe3a^{m-/p+} rats were bilaterally injected with either 3 μL of PBS (AS-Mock) or 3 μL of E6AP (AS-E6AP, 80 nM) and allowed to recover overnight before training in fear conditioning. A one foot-shock paradigm followed by a 72 h post-training contextual and cued test were utilized for this portion. A control group of wild type rats was used as a reference. No significant differences were observed during fear conditioning training in any of the groups. However, as expected at 72 h post-training, the AS-Mock injected rats had deficits in both cued and contextual fear conditioning compared to WT animals (FIG. 9A&B), which was consistent with what the inventors have reported previously (Dodge et al. 2020). Surprisingly, AS-E6AP injected animals showed a rescue in contextual fear conditioning compared to AS-Mock injected animals, with AS-E6AP rats performing similar to WT rats (FIG. 9A). Hippocampal injection of E6AP did not rescue the cued deficit as might be expected because this part of the test is more amygdala dependent (FIG. 9B).

The inventors have been active in the development of gene therapy approaches for AS. Using rAAV the inventors had initially showed that mouse Ube3a gene delivery could show a rescue of some of the AS mouse phenotype (Daily et al. 2011). This included a partial recovery of LTP deficits and rescue of Morris water maze task. The inventors have further explored the gene therapy approach, developing a vector with a human E6AP fused to a secretion signal peptide and a cell penetrating peptide (CPP). This construct was termed STUb. The concept was to develop a factory of cells in the brain that could secrete E6AP protein and have it taken up by neighboring cells with the CPP. ICV injection of equal doses of rAAV-hUBE3A and rAAV-hSTUb compared to control injected AS rats demonstrated recovery of LTP (FIG. 14). Interestingly, the hUBE3A delivery again partially recovered the LTP deficit, however, the STUb was able to fully recover the LTP back to wild type levels (FIG. 14). Initial studies failed to observe a rescue in motor deficits in AS mice with the hippocampal administration of AAV-Ube3a (Daily et al., PloS, 2017). Therefore, the inventors examined if STUb and/or hUBE3A delivery by ICV injection could potentially have benefits to motor deficits. As shown in FIG. 15, STUb but not hUBE3A has significant benefits in motor performance. The inventors previously demonstrated that AS rats have a significant hind-limb clasping phenotype (Dodge et al. 2020), this was rescued with STUb but not hUBE3A delivery (FIG. 15b). In the accelerating rotarod the inventors also saw significant improvement with STUb but not hUBE3A delivery (FIG. 15a). The inventors believe this represents the ability of the secreted STUb to become available for a greater number of neurons throughout the brain, potentially in the cerebellum, striatum and/or motor cortex. Overall, the data suggests that a secreted version of UBE3A can offer a more beneficial therapeutic for AS treatment.

Given the preceding examples and the results obtained therein, the inventors developed a secreted only E6AP that could have a significant therapeutic benefit to AS patients. This secreted E6AP may work better than the current STUB design due to its ability to maintain its presence outside the cell and thus diffuse to greater distances and cover more of the brain.

A Homo sapiens UBE3A gene was formed from H sapiens UBE3A variant 1:
(SEQ ID No: 2)
ACAGTATGAC ATCTGATGCT GGAGGGTCGC ACTTTCACAA
ATGAGTCAGC TGGTACATGG GGTTATCATC AATTTTTAGC TCTTCTGTCT
GGGAGATACA AGTTTGGAAG CAATCTTGGG GTACTTACCC ACAAGGCTGG
TGGAGACCAG ATCAGGAGAA CCTCAGTCTG ACGACATTGA AGCTAGCCGA
ATGAAGCGAG CAGCTGCAAA GCATCTAATA GAACGCTACT ACCACCAGTT
AACTGAGGGC TGTGGAAATG AAGCCTGCAC GAATGAGTTT TGTGCTTCCT
GTCCAACTTT TCTTCGTATG GATAATAATG CAGCAGCTAT TAAAGCCCTC
GAGCTTTATA AGATTAATGC AAAACTCTGT GATCCTCATC CCTCCAAGAA
AGGAGCAAGC TCAGCTTACC TTGAGAACTC GAAAGGTGCC CCCAACAACT
CCTGCTCTGA GATAAAAATG AACAAGAAAG GCGCTAGAAT TGATTTTAAA
GATGTGACTT ACTTAACAGA AGAGAAGGTA TATGAAATTC TTGAATTATG
TAGAGAAAGA GAGGATTATT CCCCTTTAAT CCGTGTTATT GGAAGAGTTT
TTTCTAGTGC TGAGGCATTG GTACAGAGCT TCCGGAAAGT TAAACAACAC
ACCAAGGAAG AACTGAAATC TCTTCAAGCA AAAGATGAAG
ACAAAGATGA GGATGAAAAG GAAAAAGCTG CATGTTCTGC TGCTGCTATG
GAAGAAGACT CAGAAGCATC TTCCTCAAGG ATAGGTGATA GCTCACAGGG
AGACAACAAT TTGCAAAAAT TAGGCCCTGA TGATGTGTCT GTGGATATTG
ATGCCATTAG AAGGGTCTAC ACCAGATTGC TCTCTAATGA AAAAATTGAA
ACTGCCTTTC TCAATGCACT TGTATATTTG TCACCTAACG TGGAATGTGA
CTTGACGTAT CACAATGTAT ACTCTCGAGA TCCTAATTAT CTGAATTTGT
TCATTATCGT AATGGAGAAT AGAAATCTCC ACAGTCCTGA ATATCTGGAA
ATGGCTTTGC CATTATTTTG CAAAGCGATG AGCAAGCTAC CCCTTGCAGC
CCAAGGAAAA CTGATCAGAC TGTGGTCTAA ATACAATGCA GACCAGATTC
GGAGAATGAT GGAGACATTT CAGCAACTTA TTACTTATAA AGTCATAAGC
AATGAATTTA ACAGTCGAAA TCTAGTGAAT GATGATGATG CCATTGTTGC
TGCTTCGAAG TGCTTGAAAA TGGTTTACTA TGCAAATGTA GTGGGAGGGG
AAGTGGACAC AAATCACAAT GAAGAAGATG ATGAAGAGCC
CATCCCTGAG TCCAGCGAGC TGACACTTCA GGAACTTTTG GGAGAAGAAA
GAAGAAACAA GAAAGGTCCT CGAGTGGACC CCCTGGAAAC TGAACTTGGT
GTTAAAACCC TGGATTGTCG AAAACCACTT ATCCCTTTTG AAGAGTTTAT
TAATGAACCA CTGAATGAGG TTCTAGAAAT GGATAAAGAT TATACTTTTT
TCAAAGTAGA AACAGAGAAC AAATTCTCTT TTATGACATG TCCCTTTATA
TTGAATGCTG TCACAAAGAA TTTGGGATTA TATTATGACA ATAGAATTCG
CATGTACAGT GAACGAAGAA TCACTGTTCT CTACAGCTTA GTTCAAGGAC
AGCAGTTGAA TCCATATTTG AGACTCAAAG TTAGACGTGA CCATATCATA
GATGATGCAC TTGTCCGGCT AGAGATGATC GCTATGGAAA ATCCTGCAGA
CTTGAAGAAG CAGTTGTATG TGGAATTTGA AGGAGAACAA GGAGTTGATG
AGGGAGGTGT TTCCAAAGAA TTTTTTCAGC TGGTTGTGGA GGAAATCTTC
AATCCAGATA TTGGTATGTT CACATACGAT GAATCTACAA AATTGTTTTG
GTTTAATCCA TCTTCTTTTG AAACTGAGGG TCAGTTTACT CTGATTGGCA
TAGTACTGGG TCTGGCTATT TACAATAACT GTATACTGGA TGTACATTTT
CCCATGGTTG TCTACAGGAA GCTAATGGGG AAAAAAGGAA CTTTTCGTGA
CTTGGGAGAC TCTCACCCAG TTCTATATCA GAGTTTAAAA GATTTATTGG
AGTATGAAGG GAATGTGGAA GATGACATGA TGATCACTTT CCAGATATCA
CAGACAGATC TTTTTGGTAA CCCAATGATG TATGATCTAA AGGAAAATGG
TGATAAAATT CCAATTACAA ATGAAAACAG GAAGGAATTT GTCAATCTTT
ATTCTGACTA CATTCTCAAT AAATCAGTAG AAAAACAGTT CAAGGCTTTT
CGGAGAGGTT TTCATATGGT GACCAATGAA TCTCCCTTAA AGTACTTATT
CAGACCAGAA GAAATTGAAT TGCTTATATG TGGAAGCCGG AATCTAGATT
TCCAAGCACT AGAAGAAACT ACAGAATATG ACGGTGGCTA TACCAGGGAC
TCTGTTCTGA TTAGGGAGTT CTGGGAAATC GTTCATTCAT TTACAGATGA
ACAGAAAAGA CTCTTCTTGC AGTTTACAAC GGGCACAGAC AGAGCACCTG
TGGGAGGACT AGGAAAATTA AAGATGATTA TAGCCAAAAA
TGGCCCAGAC ACAGAAAGGT TACCTACATC TCATACTTGC TTTAATGTGC
TTTTACTTCC GGAATACTCA AGCAAAGAAA AACTTAAAGA GAGATTGTTG
AAGGCCATCA CGTATGCCAA AGGATTTGGC ATGCTGTAAA ACAAAACAAA
ACAAAAT (BC002582.2);

The cDNA was subcloned and sequenced. The UBE3A, variant 1 gene (SEQ ID No: 2) was fused to one of three genes encoding a secretion signaling peptide, based on GDNF;

(SEQ ID No: 3)
ATGAAGTTATGGGATGTCGTGGCTGTCTGCCTGGTGCTGCTCCACACC
GCGTCCGCC (XM_017009337.2),
from insulin;
(SEQ ID No: 5)
ATGGCCCTGTGGATGCGCCTCCTGCCCCTGCTGGCGCTGCTGGCCCTCT
GGGGACCTGACCCAGCCGCAGCC (AH002844.2),
or
from IgK;
(SEQ ID No: 6)
ATGGAGACAGACACACTCCTGCTATGGGTACTGCTGCTCTGGGTTCCA
GGTTCCACTGGT (NG_000834.1).

The construct was inserted into the hSUb vector, under a CMV chicken-beta actin hybrid promoter or human ubiquitin c promoter. Woodchuck hepatitis post-transcriptional regulatory element (WPRE) is present to increase expression levels.

Other sequences of Homo sapiens UBE3A include UBE3A version 1 and variant 2, seen below;

UBE3A version 1 cDNA (AH005553.1);
(SEQ ID No: 7)
GGAGTAGTTT ACTGAGCCAC TAATCTAAAG TTTAATACTG
TGAGTGAATA CCAGTGAGTA CCTTTGTTAA TGTGGATAAC CAATACTTGG
CTATAGGAAG TTTTTTAGTT GTGTGTTTTA TNACACGTAT TTGACTTTGT
GAATAATTAT GGCTTATAAT GGCTTGTCTG TTGGTATCTA TGTATAGCGT
TTACAGTTTC CTTTAAAAAA CATGCATTGA GTTTTTTAAT AGTCCAACCC
TTAAAATAAA TGTGTTGTAT GGCCACCTGA TCTGACCACT TTCTTTCATG
TTGACATCTT TAATTTTAAA ACTGTTTTAT TTAGTGCTTA AATCTTGTTN
ACAAAATTGT CTTCCTAAGT AATATGTCTA CCTTTTTTTT TGGAATATGG
AATATTTTGC TAACTGTTTC TCAATTGCAT TTTACAGATC AGGAGAACCT
CAGTCTGACG ACATTGAAGC TAGCCGAATG TAAGTGTAAC TTGGTTGAGA
CTGTGGTTCT TATTTTGAGT TGCCCTAGAC TGCTTTAAAT TACGTCACAT
TATTTGGAAA TAATTTCTGG TTAAAAGAAA GGAATCATTT AGCAGTAAAT
GGGAGATAGG AACATACCTA CTTTTTTTCC TATCAGATAA CTCTAAACCT
CGGTAACAGT TTACTAGGTT TCTACTACTA GATAGATAAA TGCACACGCC
TAAATTCTTA GTCTTTTTGC TTCCCTGGTA GCAGTTGTAG GGAAATAGGG
AGGTTGAGGA AAGAGTTTAA CAGTCTCAAC GCCTACCATA TTTAAGGCAT
CAAGTACTAT GTTATAGATA CAGAGATGCG TAATAATTAG TTTTCACCCT
ACAGAAATTT ATATTATACT CAAGAGTGAA AGATGCAGAA GCAAATAATT
TCAGTCACTG AGGTAGAATG GTATCCAAAA TACAATAGTA ACATGAAGGA
GTACTGGAGT ACCAGGTATG CAATAGGAAT CTAGTGTAGA TGGCAGGGAA
GTAAGAGTGG CCAGGAAATG CTAAGTTCAG TCTTGAAATG TGACTGGGAA
TCAGGCAGCT ATCAACTATA AGTCAAATGT TTACAAGCTG TTAAAAATGA
AATACTGATT ATGTAAAAGA AAACCGGATT GATGCTTTAA ATAGACTCAT
TTTCNTAATG CTAATTTTTA AAATGATAGA ATCCTACAAN TCTTAGCTGT
AAACCTTGTG ATTTTTCAGC TGTTGTACTA AACAACTTAA GCACATATAC
CATCAGACAA GCCCCCNTCC CCCCTTTTAA ACCAAAGGAA TGTATACTCT
GTTAATACAG TCAGTAAGCA TTGACATTCT TTATCATAAT ATCCTAGAAA
ATATTTATTA ACTATTTCAC TAGTCAGGAG TTGTGGTAAA TAGTGCATCT
CCATTTTCTA CTTCTCATCT TCATACACAG GTTAATCACT TCAGTGCTTG
ACTAACTTTT GCCTTGATGA TATGTTGAGC TTTGTACTTG AGAGCTGTAC
TAATCACTGT GCTTATTGTT TGAATGTTTG GTACAGGAAG CGAGCAGCTG
CAAAGCATCT AATAGAACGC TACTACCACC AGTTAACTGA GGGCTGTGGA
AATGAAGCCT GCACGAATGA GTTTTGTGCT TCCTGTCCAA CTTTTCTTCG
TATGGATAAT AATGCAGCAG CTATTAAAGC CCTCGAGCTT TATAAGATTA
ATGCAAAACT CTGTGATCCT CATCCCTCCA AGAAAGGAGC AAGCTCAGCT
TACCTTGAGA ACTCGAAAGG TGCCCCCAAC AACTCCTGCT CTGAGATAAA
AATGAACAAG AAAGGCGCTA GAATTGATTT TAAAGGTAAG ATGTTTTATT
TTCAATTGAG AATTGTTGCC TGAAAACCAT GTGGGAGATT TAAATGTATT
AGTTTTTATT TGTTTTTTCT TCTGTGACAT AAAGACATTT TGATATCGTA
GAACCAATTT TTTATTGTGG TAACGGACAG GAATAATAAC TACATTTTAC
AGGTCTAATC ATTGCTAATT AGAAGCAGAT CATATGCCAA AAGTTCATTT
GTTAATAGAT TGATTTGAAC TTTTTAAAAT TCTTAGGAAA AATGTATTAA
GTGGTAGTGA ATCTCCAAAA CTATTTAAGA GCTGTATTAT GATTAATCAG
TACATGACAT ATTGGTTCAT ATTTATAATT AAAGCTATAC ATTAATAGAT
ATCTTGATTA TAAAGAAAGT TTAAACTCAT GATCTTATTA AGAGTTATAC
ATTGTTGAAA GAATGTAAAA GCATGGGTGA GGTCATTGGT ATAGGTAGGT
AGTTCATTGA AAAAAATAGG TAAGCATTAA ATTTTGTTTG CTGAATCTAA
GTATTAGATA CTTTAAGAGT TGTATATCAT AAATGATATT GAGCCTAGAA
TGTTTGGCTG TTTTACTTTT AGAACTTTTT GCAACAGAGT AAACATACAT
ATTATGAAAA TAAATGTTCT CTTTTTTCCT CTGATTTTCT AGATGTGACT
TACTTAACAG AAGAGAAGGT ATATGAAATT CTTGAATTAT GTAGAGAAAG
AGAGGATTAT TCCCCTTTAA TCCGTGTTAT TGGAAGAGTT TTTTCTAGTG
CTGAGGCATT GGTACAGAGC TTCCGGAAAG TTAAACAACA CACCAAGGAA
GAACTGAAAT CTCTTCAAGC AAAAGATGAA GACAAAGATG
AAGATGAAAA GGAAAAAGCT GCATGTTCTG CTGCTGCTAT GGAAGAAGAC
TCAGAAGCAT CTTCCTCAAG GATAGGTGAT AGCTCACAGG GAGACAACAA
TTTGCAAAAA TTAGGCCCTG ATGATGTGTC TGTGGATATT GATGCCATTA
GAAGGGTCTA CACCAGATTG CTCTCTAATG AAAAAATTGA AACTGCCTTT
CTCAATGCAC TTGTATATTT GTCACCTAAC GTGGAATGTG ACTTGACGTA
TCACAATGTA TACTCTCGAG ATCCTAATTA TCTGAATTTG TTCATTATCG
TAATGGAGAA TAGAAATCTC CACAGTCCTG AATATCTGGA AATGGCTTTG
CCATTATTTT GCAAAGCGAT GAGCAAGCTA CCCCTTGCAG CCCAAGGAAA
ACTGATCAGA CTGTGGTCTA AATACAATGC AGACCAGATT CGGAGAATGA
TGGAGACATT TCAGCAACTT ATTACTTATA AAGTCATAAG CAATGAATTT
AACAGTCGAA ATCTAGTGAA TGATGATGAT GCCATTGTTG CTGCTTCGAA
GTGCTTGAAA ATGGTTTACT ATGCAAATGT AGTGGGAGGG GAAGTGGACA
CAAATCACAA TGAAGAAGAT GATGAAGAGC CCATCCCTGA
GTCCAGCGAG CTGACACTTC AGGAACTTTT GGGAGAAGAA
AGAAGAAACA AGAAAGGTCC TCGAGTGGAC CCCCTGGAAA
CTGAACTTGG TGTTAAAACC CTGGATTGTC GAAAACCACT TATCCCTTTT
GAAGAGTTTA TTAATGAACC ACTGAATGAG GTTCTAGAAA TGGATAAAGA
TTATACTTTT TTCAAAGTAG AAACAGAGAA CAAATTCTCT TTTATGACAT
GTCCCTTTAT ATTGAATGCT GTCACAAAGA ATTTGGGATT ATATTATGAC
AATAGAATTC GCATGTACAG TGAACGAAGA ATCACTGTTC TCTACAGCTT
AGTTCAAGGA CAGCAGTTGA ATCCATATTT GAGACTCAAA GTTAGACGTG
ACCATATCAT AGATGATGCA CTTGTCCGGG TAAGTTGGGC TGCTAGATTA
AAAACCTAAT AATGGGGATA TCATGATACA GTTCAGTGAA TTCATTTTAA
AAGTGACTGA AAAAAATGAT ACCATATAGC ATAGGAACAC ATGGACATTT
CTGATCTTAT ATAAGTATTA TACTTTTGTT GTTCCTGTGC AAGTTTATAG
ATGTGTTCTA CAAAGTATCG GTTGTATTAT ATAATGGTCA TGCTATCTTT
GAAAAAGAAT GGGTTTTCTA AATCTTGAAA ACTAAATCCA AAGTTTCTTT
CATTCAGAAG AGAATAGAGT GTTGGACAAA GACCAGAACA
AGAGAAATGT GGAGATACCC AATAATAAGT GTGGATGTGC AGTCTTGAAC
TGGGAGTAAT GGTACAGTAA AACCATACCA TAAAATTATA GGTAGTGTCC
AAAAAATTCC ATCGTGTAAA ATTCAGAGTT GCATTATTGT GGACTTGAAG
AAGCAGTTGT ATGTGGGACG GTATCGATAA GCTTGATATC GAATTCCTGC
AGCCCGGGGG ATCCACTAGT GTGGTAATTA ATACTAAGTC TTACTGTGAG
AGACCATAAA CTGCTTTAGT ATTCAGTGTA TTTTTCTTAA TTGAAATATT
TAACTTATGA CTTAGTAGAT ACTAAGACTT AACCCTTGAG TTTCTATTCT
AATAAAGGAC TACTAATGAA CAATTTTGAG GTTAGACCTC TACTCCATTG
TTTTTGCTGA AATGATTTAG CTGCTTTTCC ATGTCCTGTG TAGTCCAGAC
TTAACACACA AGTAATAAAA TCTTAATTAA TTGTATGTTA ATTTCATAAC
AAATCAGTAA AGTTAGCTTT TTACTATGCT AGTGTCTGTT TTGTGTCTGT
CTTTTTGATT ATCTTTAAGA CTGAATCTTT GTCTTCACTG GCTTTTTATC
AGTTTGCTTT CTGTTTCCAT TTACATACAA AAAGTCAAAA ATTTGTATTT
GTTTCCTAAT CCTACTCCTT GTTTTTATTT TGTTTTTTTC CTGATACTAG
CAATCATCTT CTTTTCATGT TTATCTTTTC AATCACTAGC TAGAGATGAT
CGCTATGGAA AATCCTGCAG ACTTGAAGAA GCAGTIGTAT GTGGAATTTG
AAGGAGAACA AGGAGTTGAT GAGGGAGGTG TTTCCAAAGA ATTTTTTCAG
CTGGTTGTGG AGGAAATCTT CAATCCAGAT ATTGGTAAAT ACATTAGTAA
TGTGATTATG GTGTCGTATC ATCTTTTGAG TTAGTTATTT GTTTATCTTA
CTTTGTAAAT ATTTTCAGCT ATGAAGAGCA GCAAAAGAAG GATTTGGTAT
GGATTACCCA GAATCACACA TCATGACTGA ATTTGTAGGT TTTAGGAACT
GATTTGTATC ACTAATTTAT TCAAATTCTT TTATTTCTTA GAAGGAATAT
TCTAATGAAG GAAATTATCT CTTTGGTAAA CTGAATTGAA AGCACTTTAG
AATGGTATAT TGGAACAGTT GGAGGGATTT CTTTGCTTTT TGTTGTCTAA
AACCATCATC AAACTCACGG TTTTCCTGAC CTGTGAACTT CAAAGAACAA
TGGTTTGAAG AGTATTGAGA GACTGTCTCA CAAGTATGTC ATGCTCAAAG
TTCAGAAACA CTAGCTGATA TCACATTAAT TAGGTTTATT TGCTATAAGA
TTTCTTGGGG CTTAATATAN GTAGTGTTCC CCCAAACTTT TTGAACTCCA
GAACTCTTTT CTGCCCTAAC AGTAGCTACT CAGGAGCTGA GGCAGGAGAA
TTGTTTGAAC CTAGGAGGCA GAGGTTGCAG TGAGCTGAGA TCGTGCCACT
CCAGCCCACC CCTGGGTAAC AGAGCGAGAC TCCATCTCAA AGAAAAAAAT
GAAAAATTGT TTTCAAAAAT AGTACGTGTG GTACAGATAT AAGTAATTAT
ATTTTTATAA ATGAAACACT TTGGAAATGT AGCCATTTTT TGTTTTTTTA
TGTTTATTTT TCAGCTATGG GTGGATAAAG CATGAATATA ACTTTTCTTA
TGTGTTAGTA GAAAATTAGA AAGCTTGAAT TTAATTAACG TATTTTTCTA
CCCGATGCCA CCAAATTACT TACTACTTTA TTCCTTTGGC TTCATAAAAT
TACATATCAC CATTCACCCC AATTTATAGC AGATATATGT GGACATTGTT
TTCTCAAGTG CTAATATAAT AGAAATCAAT GTTGCATGCC TAATTACATA
TATTTTAAAT GTTTTATATG CATAATTATT TTAAGTTTAT ATTTGTATTA
TTCATCAGTC CTTAATAAAA TACAAAAGTA ATGTATTTTT AAAAATCATT
TCTTATAGGT ATGTTCACAT ACGATGAATC TACAAAATTG TTTTGGTTTA
ATCCATCTTC TTTTGAAACT GAGGGTCAGT TTACTCTGAT TGGCATAGTA
CTGGGTCTGG CTATTTACAA TAACTGTATA CTGGATGTAC ATTTTCCCAT
GGTTGTCTAC AGGAAGCTAA TGGGGAAAAA AGGAACTTTT CGTGACTTGG
GAGACTCTCA CCCAGTAAGT TCTTTGTCAT TTTTTTAATT CAGTCTCTTA
GATTTTATTT AAATGCAAAA ATTTAATTTA TGTCAAAATT TTAAAGTTTT
TGTTTAGAAT CTTTGTTGAT ACTCTTATCA ATAAGATAAA AATGTTTTAA
TCTGACCGAA GTACCAGAAA CACTTAAAAA CTCAAAGGGG GACATTTTTA
TATATTGCTG TCAGCACGAA GCTTTCGTAA GATTGATTTC ATAGAGAAGT
GTTTCTAAAC ATTTTGTTTG TGTTTTAGTG AAATCTTAAG AGATAGGTAA
AAATCAGAGT AGCCCTGGCT AAGGGTCTTG GTAGTTACAA CGAGTGTGCC
TGCTCCTACC ACCCCCACCC CCACCTTGAG ACACCACAGA ATTTCTCATA
GAGCACAGTG TGAATTCTAT TGCTAAATTG GTGGTATGGG GTTTCTCAGC
AGAGAATGGG ACATCACAGT GACTGACAAT CTTTCTTTTA TAGGTTGGAA
ACTATTTGGG GGACTGGAGG GATACTGTCT ACACTTTTTA CAATTTTTAT
TGATAAGATT TTTGTTGTCT TCTAAGAAGA GTGATATAAA TTATTTGTTG
TATTTTGTAG TTCTATGGTG GCCTCAATTT ACCATTTCTG GTTGCTAGGT
TCTATATCAG AGTTTAAAAG ATTTATTGGA GTATGAAGGG AATGTGGAAG
ATGACATGAT GATCACTTTC CAGATATCAC AGACAGATCT TTTTGGTAAC
CCAATGATGT ATGATCTAAA GGAAAATGGT GATAAAATTC CAATTACAAA
TGAAAACAGG AAGGTAATAA ATGTTTTTAT GTCACATTTT GTCTCTTCAT
TAACACTTTC AAAGCATGTA TGCTTATAAT TTTTAAAGAA GTATCTAATA
TAGTCTGTAC AAAAAAAAAA CAAGTAACTA AGTTTATGTA AATGCTAGAG
TCCACTTTTC TAAATCTTGG ATATAAGTTG GTATGAAAGC ACACAGTTGG
GCACTAAAGC CCCTTTTAGA GAAAGAGGAC ATGAAGCAGG
AGATAGTTAA TAGCTAAGTG TGGTTGTAGT ATAAAGCAAG AAGCAGGGTG
TTTCTTGTAT TAAGCTGTAA GCAGGAACCT CATGATTAAG GTCTTTATCA
CAGAACAAAT AAAAATTACA TTTAATTTAC ACATGTATAT CCTGTTTGTG
ATAAAAATAC ATTTCTGAAA AGTATACTTT ACGTCAGATT TGGGTTCTAT
TGACTAAAAT GTGTTCATCG GGAATGGGAA TAACCCAGAA CATAACAAGC
AAAAAATTAT GACAAATATA TAGTATACCT TTAAGAAACA TGTTTATATT
GATATAATTT TTTGATTAAA TATTATACAC ACTAAGGGTA CAANGCACAT
TTTCCTTTTA TGANTTNGAT ACAGTAGTTT ATGTGTCAGT CAGATACTTC
CACATTTTTG CTGAACTGGA TACAGTAAGC AGCTTACCAA ATATTCTATG
GTAGAAAACT NGGACTTCCT GGTTTGCTTA AATCAAATAT ATTGTACTCT
CTTAAAACGG TTGGCATTTA TAAATAGATG GATACATGGT TTAAATGTGT
CTGTTNACAT ACCTAGTTGA GAGAACCTAA AGAATTTTCT GCGTCTCCAG
CATTTATATT CAGTTCTGTT TAATACATTA TCGAAATTGA CATTTATAAG
TATGACAGTT TTGTGTATAT GGCCTTTTCA TAGCTTAATA TTGGCTGTAA
CAGAGAATTG TGAAATTGTA AGAAGTAGTT TTCTTTGTAG GTGTAAAATT
GAATTTTTAA GAATATTCTT GACAGTTTTA TGTATATGGC CTTTTCATAG
CTTAATATTG GCTATAACAG AGAATTGTGA AATTGTTAAG AAGTAGGTGT
AAAATTGAAT TTTTAAGAAT ATTCTTGAAT GTTTTTTTCT TGGAAAAATT
AAAAAGCTAT GCAGCCCAAT AACTTGTGTT TTGTTTGCAT AGCATATTAT
AAGAAGTTCT TGTGATTAAT GTTTTCTACA GGAATTTGTC AATCTTTATT
CTGACTACAT TCTCAATAAA TCAGTAGAAA AACAGTTCAA GGCTTTTCGG
AGAGGTTTTC ATATGGTGAC CAATGAATCT CCCTTAAAGT ACTTATTCAG
ACCAGAAGAA ATTGAATTGC TTATATGTGG AAGCCGGGTA
AGAAAGCAGG TGTCTGCAAA AAGTCATGTA TCGATTTATT GTTTGTAATG
ATACAGTAGT ATAGCAGATA ACTAAGACAT ATTTTCTTGA ATTTGCAGAA
TCTAGATTTC CAAGCACTAG AAGAAACTAC AGAATATGAC GGTGGCTATA
CCAGGGACTC TGTTCTGATT AGGTGAGGTA CTTAGTTCTT CAGAGGAAGA
TTTGATTCAC CAAAGGGGTG TGTGATTTTG CTTCAGACCT TTATCTCTAG
GTACTAATTC CCAAATAAGC AAACTCACAA ATTGTCATCT ATATACTTAG
ATTTGTATTT GTAATATAAT CACCATTTTT CAGAGCTAAT CTTGTGATTT
ATTTCATGAA TGAAGTGTTG TTATATATAA GTCTCATGTA ATCTCCTGCA
TTTGGCGTAT GGATTATCTA GTATTCCTCA CTGGTTAGAG TATGCTTACT
GCTGGTTAGA AGATAATTAA AATAAGGCTA CCATGTCTGC AATTTTTCCT
TTCTTTTGAA CTCTGCATTT GTGAACTGTT ACATGGCTTC CCAGGATCAA
GCACTTTTTG AGTGAAATGG TAGTCTTTTA TTTAATTCTT AAGATAATAT
GTCCAGATAC ATACTAGTAT TTCCATTTTA CACCCTAAAA AACTAAGCCC
TGAATTCTCA CAGAAAGATG TAGAGGTTCC CAGTTCTATC TGCTTTTAAA
CAAATGCCCT TACTACTCTA CTGTCTACTT CTGTGTACTA CATCATCGTA
TGTAGTTGTT TGCATTTGGG CCAGTTGGTT GGGGCAGGGG TCTTTTTTTC
TTTTGTCCCT TAATCTGTAT CACTTTTTCC TCCCAAAGTT GAGTTAAAGG
ATGAGTAGAC CAGGAGAATA AAGGAGAAAG GATAAATAAA
ATATATACCC AAAGGCACCT GGAGTTAATT TTTCCAAATA TTCATTTCAG
TCTTTTTCAA TTCATAGGAT TTTGTCTTTT GCTCATTACT GACTGCATAA
TGTGATTATA CCATAGTTTA AATAGTCACT TCCTGTTACT ACACACTTGG
GTTTTCTCAA TTTTTTACTA TTGTAGTACT AATATTTTAC TATATTGTAA
TCTAATCCAA ATTTTTACGT ATTCAGAGCT GTTCAGGATA AATTTGCTTG
GAAATTTTTA AATCACCAGA AGTGATACTA TCCTGATAAT TAACTTCCAA
GTTGTCTCTT AATATAGTTT TAATGCAAAT CATAAGCTTA TGTTAGTACC
AGTCATAATG AATGCCAAAC TGAAACCAGT ATTGTATTTT TTCTCATTAG
GGAGTTCTGG GAAATCGTTC ATTCATTTAC AGATGAACAG AAAAGACTCT
TCTTGCAGTT TACAACGGGC ACAGACAGAG CACCTGTGGG AGGACTAGGA
AAATTAAAGA TGATTATAGC CAAAAATGGC CCAGACACAG
AAAGGTAGGT AATTATTAAC TTGTGACTGT ATACCTACCG AAAACCTTGC
ATTCCTCGTC ACATACATAT GAACTGTCTT TATAGTTTCT GAGCACATTC
GTGATTTTAT ATACAAATCC CCAAATCATA TTAGACAATT GAGAAAATAC
TTTGCTGTCA TTGTGTGAGG AAACTTTTAA GAAATTGCCC TAGTTAAAAA
TTATTATGGG GCTCACATTG GTTTGGAATC AAATTAGTGT GATTCATTTA
CTTTTTTGAT TCCCAGCTTG TTAATTGAAA GCCATATAAC ATGATCATCT
ATTTAGAATG GTTACATTGA GGCTCGGAAG ATTATCATTT GATTGTGCTA
GAATCCTGTT ATCAAATCAT TTTCTTAGTC ATATTGCCAG CAGTGTTTCT
AATAAGCATT TAAGAGCACA CACTTTGCAG TCTTGTAAAA CAGGTTTGAG
TATTTTCTCC ACCTTAGAGG AAGTTACTTG ACTTCTCAGT GACCTAACCT
CTAAAGTGCA TTTACTGATG TCCTCTCTGT GGTTTTGTTG TGGAAAGATT
TAGTTAAATG AACTGTAAGA ATTCAGTACC TAAAATGGTA TCTGTTATGT
AGTAAAAACT CAATGGATAC AGTATCTTAT CATCGTCACT AGCTTTGAGT
AATTTATAGG ATAAAGGCAA CTTGGTAGTT ACACAACAAA AAGTTTATGA
TTTGCATTAA TGTATAGTTT GCATTGCAGA CCGTCTCAAC TATATACAAT
CTAAAAATAG GAGCATTTAA TTCTAAGTGT ATTTCCCATG ACTTACAGTT
TTCCTGTTTT TTTCCCCTTT TCTCTATTTA GGTTACCTAC ATCTCATACT
TGCTTTAATG TGCTTTTACT TCCGGAATAC TCAAGCAAAG AAAAACTTAA
AGAGAGATTG TTGAAGGCCA TCACGTATGC CAAAGGATTT GGCATGCTGT
AAAACAAAAC AAAACAAAAT AAAACAAAAA AAAGGAAGGA
AAAAAAAAGA AAAAATTTAA AAAATTTTAA AAATATAACG
AGGGATAAAT TTT (AH005553.1), which encodes for;
(SEQ ID No: 8)
MKRAAAKHLIERYYHQLTEGCGNEACTNEFCASCPTFLRMDNNAAAIKA
LELYKINAKLCDPHPSKKGASSAYLENSKGAPNNSCSEIKMNKKGARIDFKDVT
YLTEEKVYEILELCREREDYSPLIRVIGRVFSSAEALVQSFRKVKQHTKEELKSL
QAKDEDKDEDEKEKAACSAAAMEEDSEASSSRIGDSSQGDNNLQKLGPDDVS
VDIDAIRRVYTRLLSNEKIETAFLNALVYLSPNVECDLTYHNVYSRDPNYLNLFI
IVMENRNLHSPEYLEMALPLFCKAMSKLPLAAQGKLIRLWSKYNADQIRRMME
TFQQLITYKVISNEFNSRNLVNDDDAIVAASKCLKMVYYANVVGGEVDTNHNE
EDDEEPIPESSELTLQELLGEERRNKKGPRVDPLETELGVKTLDCRKPLIPFEEFI
NEPLNEVLEMDKDYTFFKVETENKFSFMTCPFILNAVTKNLGLYYDNRIRMYSE
RRITVLYSLVQGQQLNPYLRLKVRRDHIIDDALVRLEMIAMENPADLKKQLYV
EFEGEQGVDEGGVSKEFFQLVVEEIFNPDIGMFTYDESTKLFWFNPSSFETEGQF
TLIGIVLGLAIYNNCILDVHFPMVVYRKLMGKKGTFRDLGDSHPVLYQSLKDLL
EYEGNVEDDMMITFQISQTDLFGNPMMYDLKENGDKIPITNENRKEFVNLYSD
YILNKSVEKQFKAFRRGFHMVTNESPLKYLFRPEEIELLICGSRNLDFQALEETT
EYDGGYTRDSVLIREFWEIVHSFTDEQKRLFLQFTTGTDRAPVGGLGKLKMIIA
KNGPDTERLPTSHTCFNVLLLPEYSSKEKLKERLLKAITYAKGFGML (NP_570853.1).
H sapiens UBE3A variant 2;
(SEQ ID No: 9)
AGCCAGTCCT CCCGTCTTGC GCCGCGGCCG CGAGATCCGT
GTGTCTCCCA AGATGGTGGC GCTGGGCTCG GGGTGACTAC AGGAGACGAC
GGGGCCTTTT CCCTTCGCCA GGACCCGACA CACCAGGCTT CGCTCGCTCG
CGCACCCCTC CGCCGCGTAG CCATCCGCCA GCGCGGGCGC CCGCCATCCG
CCGCCTACTT ACGCTTCACC TCTGCCGACC CGGCGCGCTC GGCTGCGGGC
GGCGGCGCCT CCTTCGGCTC CTCCTCGGAA TAGCTCGCGG CCTGTAGCCC
CTGGCAGGAG GGCCCCTCAG CCCCCCGGTG TGGACAGGCA GCGGCGGCTG
GCGACGAACG CCGGGATTTC GGCGGCCCCG GCGCTCCCTT TCCCGGCCTC
GTTTTCCGGA TAAGGAAGCG CGGGTCCCGC ATGAGCCCCG GCGGTGGCGG
CAGCGAAAGA GAACGAGGCG GTGGCGGGCG GAGGCGGCGG
GCGAGGGCGA CTACGACCAG TGAGGCGGCC GCCGCAGCCC
AGGCGCGGGG GCGACGACAG GTTAAAAATC TGTAAGAGCC TGATTTTAGA
ATTCACCAGC TCCTCAGAAG TTTGGCGAAA TATGAGTTAT TAAGCCTACG
CTCAGATCAA GGTAGCAGCT AGACTGGTGT GACAACCTGT TTTTAATCAG
TGACTCAAAG CTGTGATCAC CCTGATGTCA CCGAATGGCC ACAGCTTGTA
AAAGAGAGTT ACAGTGGAGG TAAAAGGAGT GGCTTGCAGG
ATGGAGAAGC TGCACCAGTG TTATTGGAAA TCAGGAGAAC CTCAGTCTGA
CGACATTGAA GCTAGCCGAA TGAAGCGAGC AGCTGCAAAG CATCTAATAG
AACGCTACTA CCACCAGTTA ACTGAGGGCT GTGGAAATGA AGCCTGCACG
AATGAGTTTT GTGCTTCCTG TCCAACTTTT CTTCGTATGG ATAATAATGC
AGCAGCTATT AAAGCCCTCG AGCTTTATAA GATTAATGCA AAACTCTGTG
ATCCTCATCC CTCCAAGAAA GGAGCAAGCT CAGCTTACCT TGAGAACTCG
AAAGGTGCCC CCAACAACTC CTGCTCTGAG ATAAAAATGA
ACAAGAAAGG CGCTAGAATT GATTTTAAAG ATGTGACTTA CTTAACAGAA
GAGAAGGTAT ATGAAATTCT TGAATTATGT AGAGAAAGAG AGGATTATTC
CCCTTTAATC CGTGTTATTG GAAGAGTTTT TTCTAGTGCT GAGGCATTGG
TACAGAGCTT CCGGAAAGTT AAACAACACA CCAAGGAAGA ACTGAAATCT
CTTCAAGCAA AAGATGAAGA CAAAGATGAA GATGAAAAGG
AAAAAGCTGC ATGTTCTGCT GCTGCTATGG AAGAAGACTC AGAAGCATCT
TCCTCAAGGA TAGGTGATAG CTCACAGGGA GACAACAATT TGCAAAAATT
AGGCCCTGAT GATGTGTCTG TGGATATTGA TGCCATTAGA AGGGTCTACA
CCAGATTGCT CTCTAATGAA AAAATTGAAA CTGCCTTTCT CAATGCACTT
GTATATTTGT CACCTAACGT GGAATGTGAC TTGACGTATC ACAATGTATA
CTCTCGAGAT CCTAATTATC TGAATTTGTT CATTATCGTA ATGGAGAATA
GAAATCTCCA CAGTCCTGAA TATCTGGAAA TGGCTTTGCC ATTATTTTGC
AAAGCGATGA GCAAGCTACC CCTTGCAGCC CAAGGAAAAC TGATCAGACT
GTGGTCTAAA TACAATGCAG ACCAGATTCG GAGAATGATG GAGACATTTC
AGCAACTTAT TACTTATAAA GTCATAAGCA ATGAATTTAA CAGTCGAAAT
CTAGTGAATG ATGATGATGC CATTGTTGCT GCTTCGAAGT GCTTGAAAAT
GGTTTACTAT GCAAATGTAG TGGGAGGGGA AGTGGACACA AATCACAATG
AAGAAGATGA TGAAGAGCCC ATCCCTGAGT CCAGCGAGCT GACACTTCAG
GAACTTTTGG GAGAAGAAAG AAGAAACAAG AAAGGTCCTC
GAGTGGACCC CCTGGAAACT GAACTTGGTG TTAAAACCCT GGATTGTCGA
AAACCACTTA TCCCTTTTGA AGAGTTTATT AATGAACCAC TGAATGAGGT
TCTAGAAATG GATAAAGATT ATACTTTTTT CAAAGTAGAA ACAGAGAACA
AATTCTCTTT TATGACATGT CCCTTTATAT TGAATGCTGT CACAAAGAAT
TTGGGATTAT ATTATGACAA TAGAATTCGC ATGTACAGTG AACGAAGAAT
CACTGTTCTC TACAGCTTAG TTCAAGGACA GCAGTTGAAT CCATATTTGA
GACTCAAAGT TAGACGTGAC CATATCATAG ATGATGCACT TGTCCGGCTA
GAGATGATCG CTATGGAAAA TCCTGCAGAC TTGAAGAAGC AGTTGTATGT
GGAATTTGAA GGAGAACAAG GAGTTGATGA GGGAGGTGTT
TCCAAAGAAT TTTTTCAGCT GGTTGTGGAG GAAATCTTCA ATCCAGATAT
TGGTATGTTC ACATACGATG AATCTACAAA ATTGTTTTGG TTTAATCCAT
CTTCTTTTGA AACTGAGGGT CAGTTTACTC TGATTGGCAT AGTACTGGGT
CTGGCTATTT ACAATAACTG TATACTGGAT GTACATTTTC CCATGGTTGT
CTACAGGAAG CTAATGGGGA AAAAAGGAAC TTTTCGTGAC TTGGGAGACT
CTCACCCAGT TCTATATCAG AGTTTAAAAG ATTTATTGGA GTATGAAGGG
AATGTGGAAG ATGACATGAT GATCACTTTC CAGATATCAC AGACAGATCT
TTTTGGTAAC CCAATGATGT ATGATCTAAA GGAAAATGGT GATAAAATTC
CAATTACAAA TGAAAACAGG AAGGAATTTG TCAATCTTTA TTCTGACTAC
ATTCTCAATA AATCAGTAGA AAAACAGTTC AAGGCTTTTC GGAGAGGTTT
TCATATGGTG ACCAATGAAT CTCCCTTAAA GTACTTATTC AGACCAGAAG
AAATTGAATT GCTTATATGT GGAAGCCGGA ATCTAGATTT CCAAGCACTA
GAAGAAACTA CAGAATATGA CGGTGGCTAT ACCAGGGACT CTGTTCTGAT
TAGGGAGTTC TGGGAAATCG TTCATTCATT TACAGATGAA CAGAAAAGAC
TCTTCTTGCA GTTTACAACG GGCACAGACA GAGCACCTGT GGGAGGACTA
GGAAAATTAA AGATGATTAT AGCCAAAAAT GGCCCAGACA
CAGAAAGGTT ACCTACATCT CATACTTGCT TTAATGTGCT TTTACTTCCG
GAATACTCAA GCAAAGAAAA ACTTAAAGAG AGATTGTTGA
AGGCCATCAC GTATGCCAAA GGATTTGGCA TGCTGTAAAA CAAAACAAAA
CAAAATAAAA CAAAAAAAAG GAAGGAAAAA AAAAGAAAAA
ATTTAAAAAA TTTTAAAAAT ATAACGAGGG ATAAATTTTT GGTGGTGATA
GTGTCCCAGT ACAAAAAGGC TGTAAGATAG TCAACCACAG TAGTCACCTA
TGTCTGTGCC TCCCTTCTTT ATTGGGGACA TGTGGGCTGG AACAGCAGAT
TTCAGCTACA TATATGAACA AATCCTTTAT TATTATTATA ATTATTTTTT
TGCGTGAAAG TGTTACATAT TCTTTCACTT GTATGTACAG AGAGGTTTTT
CTGAATATTT ATTTTAAGGG TTAAATCACT TTTGCTTGTG TTTATTACTG
CTTGAGGTTG AGCCTTTTGA GTATTTAAAA AATATATACC AACAGAACTA
CTCTCCCAAG GAAAATATTG CCACCATTTG TAGACCACGT AACCTTCAAG
TATGTGCTAC TTTTTTGTCC CTGTATCTAA CTCAAATCAG GAACTGTATT
TTTTTTAATG ATTTGCTTTT GAAACTTGAA GTCTTGAAAA CAGTGTGATG
CAATTACTGC TGTTCTAGCC CCCAAAGAGT TTTCTGTGCA AAATCTTGAG
AATCAATCAA TAAAGAAAGA TGGAAGGAAG GGAGAAATTG
GAATGTTTTA ACTGCAGCCC TCAGAACTTT AGTAACAGCA CAACAAATTA
AAAACAAAAA CAACTCATGC CACAGTATGT CGTCTTCATG TGTCTTGCAA
TGAACTGTTT CAGTAGCCAA TCCTCTTTCT TAGTATATGA AAGGACAGGG
ATTTTTGTTC TTGTTGTTCT CGTTGTTGTT TTAAGTTTAC TGGGGAAAGT
GCATTTGGCC AAATGAAATG GTAGTCAAGC CTATTGCAAC AAAGTTAGGA
AGTTTGTTGT TTGTTTATTA TAAACAAAAA GCATGTGAAA GTGCACTTAA
GATAGAGTTT TTATTAATTA CTTACTTATT ACCTAGATTT TAAATAGACA
ATCCAAAGTC TCCCCTTCGT GTTGCCATCA TCTTGTTGAA TCAGCCATTT
TATCGAGGCA CGTGATCAGT GTTGCAACAT AATGAAAAAG ATGGCTACTG
TGCCTTGTGT TACTTAATCA TACAGTAAGC TGACCTGGAA ATGAATGAAA
CTATTACTCC TAAGAATTAC ATTGTATAGC CCCACAGATT AAATTTAATT
AATTAATTCA AAACATGTTA AACGTTACTT TCATGTACTA TGGAAAAGTA
CAAGTAGGTT TACATTACTG ATTTCCAGAA GTAAGTAGTT TCCCCTTTCC
TAGTCTTCTG TGTATGTGAT GTTGTTAATT TCTTTTATTG CATTATAAAA
TAAAAGGATT ATGTATTTTT AACTAAGGTG AGACATTGAT ATATCCTTTT
GCTACAAGCT ATAGCTAATG TGCTGAGCTT GTGCCTTGGT GATTGATTGA
TTGATTGACT GATTGTTTTA ACTGATTACT GTAGATCAAC CTGATGATTT
GTTTGTTTGA AATTGGCAGG AAAAATGCAG CTTTCAAATC ATTGGGGGGA
GAAAAAGGAT GTCTTTCAGG ATTATTTTAA TTAATTTTTT TCATAATTGA
GACAGAACTG TTTGTTATGT ACCATAATGC TAAATAAAAC TGTGGCACTT
TTCACCATAA TTTAATTTAG TGGAAAAAGA AGACAATGCT TTCCATATTG
TGATAAGGTA ACATGGGGTT TTTCTGGGCC AGCCTTTAGA ACACTGTTAG
GGTACATACG CTACCTTGAT GAAAGGGACC TTCGTGCAAC TGTAGTCATC
TTAAAGGCTT CTCATCCACT GTGCTTCTTA ATGTGTAATT AAAGTGAGGA
GAAATTAAAT ACTCTGAGGG CGTTTTATAT AATAAATTCG TGAAGA
(NM_000462.4), which encodes the protein:
(SEQ ID No: 10)
MEKLHQCYWK SGEPQSDDIE ASRMKRAAAK HLIERYYHQL
TEGCGNEACT NEFCASCPTF LRMDNNAAAI KALELYKINA KLCDPHPSKK
GASSAYLENS KGAPNNSCSE IKMNKKGARI DFKDVTYLTE EKVYEILELC
REREDYSPLI RVIGRVFSSA EALVQSFRKV KQHTKEELKS LQAKDEDKDE
DEKEKAACSA AAMEEDSEAS SSRIGDSSQG DNNLQKLGPD DVSVDIDAIR
RVYTRLLSNE KIETAFLNAL VYLSPNVECD LTYHNVYSRD PNYLNLFIIV
MENRNLHSPE YLEMALPLFC KAMSKLPLAA QGKLIRLWSK YNADQIRRMM
ETFQQLITYK VISNEFNSRN LVNDDDAIVA ASKCLKMVYY ANVVGGEVDT
NHNEEDDEEP IPESSELTLQ ELLGEERRNK KGPRVDPLET ELGVKTLDCR
KPLIPFEEFI NEPLNEVLEM DKDYTFFKVE TENKFSFMTC PFILNAVTKN
LGLYYDNRIR MYSERRITVL YSLVQGQQLN PYLRLKVRRD HIIDDALVRL
EMIAMENPAD LKKQLYVEFE GEQGVDEGGV SKEFFQLVVE EIFNPDIGMF
TYDESTKLFW FNPSSFETEG QFTLIGIVLG LAIYNNCILD VHFPMVVYRK
LMGKKGTFRD LGDSHPVLYQ SLKDLLEYEG NVEDDMMITF QISQTDLFGN
PMMYDLKENG DKIPITNENR KEFVNLYSDY ILNKSVEKQF KAFRRGFHMV
TNESPLKYLF RPEEIELLIC GSRNLDFQAL EETTEYDGGY TRDSVLIREF
WEIVHSFTDE QKRLFLQFTT GTDRAPVGGL GKLKMIIAKN GPDTERLPTS
HTCFNVLLLP EYSSKEKLKE RLLKAITYAK GFGML (NP_000453.2).

### Example 5—In Vivo Protein Replacement Therapy Using hSUb with Rat (Prophetic)

A similar protocol to that shown in Example 3 is conducted with hSUb to determine if motor and behavioral deficits in AS rats can be improved.

### Materials and Methods

### Animals

Ube3a deletion rats are bred and maintained as described previously [Dodge et al. 2020]. 129-Ube3a^{tm1Alb}/J mice [Jiang et al. 1998] are bred and maintained as described previously [Daily et al. 2011]. AS mice are used for initial characterization of the electrophysiological testing before moving to the rat model for more in depth testing. Male and female animals are housed in a standard 12-hr light/dark cycle and supplied with food and water ad libitum at the University of South Florida and are group-housed. The investigators are blind to treatments for all behavior testing. All procedures are conducted in compliance with the NIH Guidelines for the Care and Use of Laboratory Animals and approved by the Institutional Animal Care and Use Committee of USF (approval number A4100-01).

### Construct Generation and Vector Production

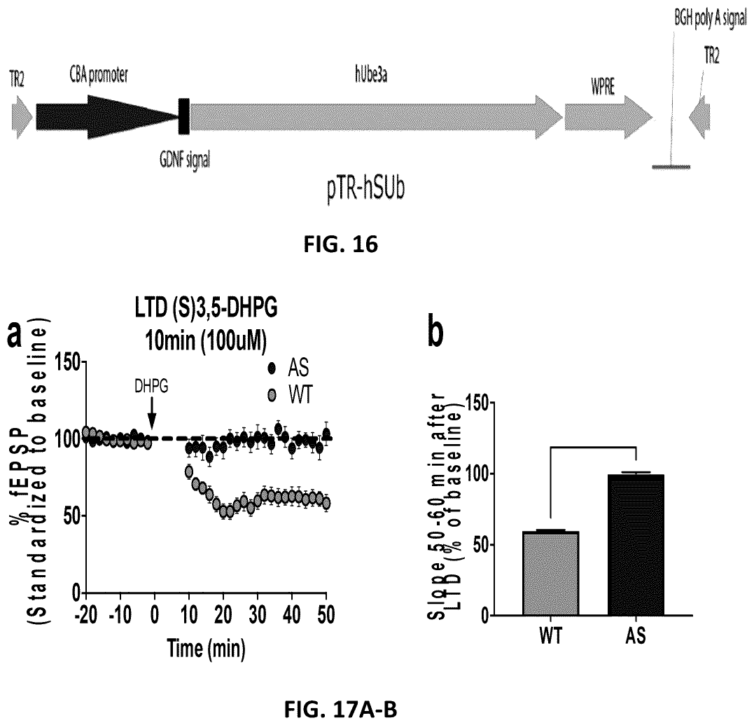

The human UBE3A gene variant 1 (GenBank: BC002582.2) (SEQ ID NO: 2) is cloned into the rAAV expression vector pTR12.1-MCSW. This vector has AAV2 terminal repeats, CBA/CAG promoter, WPRE and BGH poly signal. Starting with the hUBE3A construct, a secretion sequence (ATGAAGTTATGGGATGTCGTGGCTGTCTGCCTGGTGCTGCTCCACACCGCG TCCGCC) (SEQ ID NO: 3) is added in frame on the 5′ end of hUBE3A (FIG. 16). For in vitro testing, an HA tag is added to the vector in order to easily detect the protein. For the remaining work, the HA-tag free vector is used. In vitro testing is performed by transfecting HEK293 cells with the SUB construct using lipofectamine and incubating for 72 h before media is collected for further testing via western blot. Viral particles are generated as previously described [Daily et al. 2011; Burger et al. 2016]. Briefly, HEK293 cells are co-transfected with either the pTR-hUBE3A plasmid or the pTR-hSUB plasmid, pXX6 helper plasmid and pAAV9 serotype plasmid using polyethyleneimine transfection. Virus is harvested from cells and purified using an iodixanol gradient and concentrated using centrifugal filtration. Viral titer is determined with a dot blot assay as described previously [Burger et a; 2016]. Vectors are described as vector genomes (vg)/mL.

### Western Blotting:

Samples are heated at 95° C. for 5 minutes before running on an SDS-PAGE gel with Laemmli sample buffer containing β-mercaptoethanol. Protein is transferred to a nitrocellulose membrane using the Trans-Blot Turbo system (Bio-Rad) and REVERT stain (Li-Cor Biosciences) is used to quantify total protein before blocking with Intercept T20 blocking buffer (Li-Cor Biosciences). Blots are incubated overnight at 4° C. with gentle rocking. For UBE3A detection, an anti-UBE3A primary antibody solution (Sigma-Aldrich, SAB1404508) at 1:1000 dilution is used. For anti-HA detection, the Roche antibody clone 3F10 at 1:1000 dilution is used. Blots are incubated in secondary antibody (Li-Cor Biosciences, IgG-800CW) at 1:10,000 for 2 hr at room temperature. Detection is performed using the Odyssey CLx scanner (Li-Cor Biosciences). Analysis is performed using LI-COR Image Studio. Data are shown as the ratio of UBE3A band intensity/total protein intensity (REVERT).

### Immunohistochemical (IHC):

To test for expression of the newly generated hSUB construct in vivo IHC staining is performed. Animals are anesthetized with a lethal dose of ketamine/xylazine then are perfused with PBS before removal of the brain and submersion of the brain in cold 4% PFA. Tissue is fixed overnight before being incubated in 30% sucrose for 3 days. Sections are obtained with a microtome. Tissue sections are processed using the free floating IHC procedure with nickel and diaminobenzidine. Endogenous peroxidases are quenched with 0.3% hydrogen peroxide for 30 min. Blocking solution (5% horse serum, 0.25% Triton-X100 in PBS) is applied for one hour. Tissue sections are then incubated overnight with anti-UBE3A antibody at a 1:3000 dilution (Sigma-Aldrich, SAB1404508). After washing with PBS, secondary antibody (horse anti-mouse IgG(H+L), peroxidase, Vector Labs) is applied for one hour at room temperature. Color is developed using diaminobenzidine and nickel chloride with hydrogen peroxide. Tissue is mounted on glass slides before dehydration and cover slipping using DPX. Slides are scanned with the Zeiss Axioscan and analyzed using NearCyte. Data is shown as percent area positive DAB staining of the hippocampus normalized to the AS control group. One way ANOVA used to analyze data followed by Tukey's post-hoc test.

### Animal Injection:

Animals are anesthetized using isoflurane and positioned in a World Precision Instruments stereotactic surgery apparatus. An incision is made on the sagittal surface of the skull. AS mice are injected into the hippocampus (HPC) (X±2.7, Y-2.7, Z-3.0 from bregma) with 2 mL of either rAAV9-GFP or rAAV9-SUB at 1.0×10¹³ vg/ml. Wild type littermate controls are injected with 2 mL per site with sterile PBS (n=3M). For rats, the coordinates (X=±1.5, Y=−0.5, Z=−4.3 from bregma) are used for bilateral ICV injections and (X=4.6, Y=−6.0, Z=−5.0 from bregma) for unilateral intrahippocampal (IHPC) injections into the right hippocampus. A 10 mL volume for ICV and 2 mL volume for IHPC injections (per injection site) are applied at the coordinates above using convection enhanced delivery method [Nash et al. 2016; Carty et al. 2013]. Vectors are injected into AS rats at the following concentrations: hippocampal SUB, 3.4×10¹³ vg/ml; hippocampal UBE3A, 2×10¹³ (SD+/−0.56×10¹³) vg/ml; ICV SUB, 3.0×10¹³ (SD+/−0.58×10¹³) vg/ml; ICV UBE3A, 2×10¹³ vg/ml.

### Motor Deficit Testing:

Motor deficits are tested in rats using the hind limb clasping test along with the accelerating rotarod. Testing is performed as previously reported [Dodge et al. 2020]. Hind limb clasping is performed by suspending the animal by the tail for 30 seconds and observing the hind limbs. The animal behavior is recorded and later scored by a researcher blind to animal treatment. The clasping phenotype is measured according to the commonly used 0-3 scale with 0 corresponding with no clasping, 1 with one withdrawn limb, 2 with both limbs withdrawn, and 3 with clasping together of the hind paws. Rotarod is performed by placing the animals onto an accelerating rotating cylinder (4-40 rpm, Ugo Basile) for up to 5 minutes and the latency to fall recorded. Testing is performed for 4 trials each day over two consecutive days with 30 minutes between each trial.

### Testing of Associative Learning:

Associative learning is tested using contextual fear conditioning as previously reported [Dodge et al. 2020]. For conditioning, animals are placed in a sound attenuation chamber and allowed to acclimate for 2 minutes. The conditioned stimulus tone (95 dB, 5000 Hz) plays for 30 seconds with the animals receiving a 1 mA foot shock during the last two seconds. Animals then remain in the chamber for 3 minutes while freezing behavior (lack of movement for two consecutive seconds) is recorded. For the contextual testing, animals are placed back into the chamber 72 h after conditioning and freezing behavior is recorded. Freezing behavior is analyzed through tracking software (Ethovision XT).

### Electrophysiological Testing:

Mouse hippocampal long-term potentiation (LTP) is performed as described previously [Daily et al. 2011]. Rat hippocampal LTP testing is performed on both IHPC and ICV cohorts. LTP is tested as previously reported with minor modifications [Weeber et al. 2003]. Briefly, the brain is quickly removed and immediately submerged in 95:5 O₂: CO2 saturated ice cold cutting solution. Sections are obtained using a vibrating microtome. Hippocampi are dissected and allowed to rest in a solution of 50:50 cutting solution: artificial cerebrospinal fluid (ACSF). Slices are transferred to the recording chamber and allowed to recover for 1 h 30 m. Stimulation is applied to Schaffer collaterals using a concentric bipolar electrode (WPI) and extracellular field recordings are obtained from neurons of the CA1. To induce LTP, theta burst stimulation, consisting of five trains of four pulses at 100 Hz with an inter-burst interval of 20 seconds, is applied at an intensity that resulted in ˜50% maximal response as obtained from an input/output curve. Data are analyzed using ClampFit 10.7 software. For analysis, six sweeps over two minutes are averaged, and data are normalized to the averaged value of the initial slope of the fEPSP from the 20-minute baseline recording. Data are presented as two minute average timepoints. All data are represented mean±SEM.

### Statistical Analysis:

Graphs are created using GraphPad Prism with data presented as mean with standard error of the mean. Comparison between two groups is done by t-test. For multiple groups, data is analyzed by one-way ANOVA followed by Tukey's multiple comparison test or mixed-effects ANOVA and Fisher's LSD. P value of 0.05 is chosen as the cutoff for significance. Significance is represented as follows: *p<0.05, **p<0.01, ***p<0.001, and ****p<0.0001.

AS rats exhibit rescue of the motor deficits with the injection of rAAV-SUB. The animals also exhibit rescue of behavioral functions.

### Example 6—Human Protein Replacement Therapy with hSUb (Prophetic)

A human child presents with severe developmental delay that becomes apparent around the age of 10 months. The child later presents with absent speech, hypotonia, ataxia and microcephaly. The child moves with a jerky, puppet like gait and displays an unusually happy demeanor that is accompanied by laughing spells. The child has dysmorphic facial features characterized by a prominent chin, an unusually wide smile and deep-set eyes. The child is diagnosed with Angelman's Syndrome.

The child is treated with a therapeutically effective amount of hSUB UBE3A vector which is injected bilaterally into the left and right hippocampal hemispheres of the brain. In some cases, the vector may be injected unilaterally into one hippocampal hemisphere. Alternatively, or in addition to the hippocampal injections, the vector may be injected into the cerebral ventricles, Improvement is seen in the symptoms after treatment with a decrease in seizures, increased muscle tone, increased coordination of muscle movement and improvement in speech.

The hSUB vector is formed from cDNA cloned from a Homo sapiens UBE3A gene. The UBE3A, variant 1 gene (SEQ ID No: 2) is fused to a gene encoding a secretion signaling peptide, in this case GDNF, although insulin or IgK may also be used. The construct is inserted into the hSUb vector, under a CMV chicken-beta actin hybrid promoter or human ubiquitin c promoter. Woodchuck hepatitis post-transcriptional regulatory element (WPRE) is present to increase expression levels.

The human hSUB UBE3A vector is then transformed into E. coli using a heat shock method. The transformed E. coli were expanded in broth containing ampicillin to select for the vector and collect large amounts of vector.

In the preceding specification, all documents, acts, or information disclosed does not constitute an admission that the document, act, or information of any combination thereof was publicly available, known to the public, part of the general knowledge in the art, or was known to be relevant to solve any problem at the time of priority.

The disclosures of all publications cited above are expressly incorporated herein by reference, each in its entirety, to the same extent as if each were incorporated by reference individually.

While there has been described and illustrated specific embodiments of a method of treating UBE3A deficiencies, it will be apparent to those skilled in the art that variations and modifications are possible without deviating from the broad spirit and principle of the present invention. It is also to be understood that the following claims are intended to cover all of the generic and specific features of the invention herein described, and all statements of the scope of the invention which, as a matter of language, might be said to fall therebetween.

What is claimed is:

1. A therapeutically effective amount of a secreted ubiquitin protein ligase E3A (UBE3A) adeno-associated viral (AAV) vector for improving motor function, associative learning, or memory deficits associated with a UBE3A deficiency disease, the vector comprising:
a transcription initiation sequence;
a UBE3A sequence disposed downstream of the transcription initiation sequence, wherein the UBE3A sequence is a cDNA of SEQ ID NO: 1, SEQ ID NO: 2, SEQ ID NO: 7, a cDNA of SEQ ID NO: 8, SEQ ID NO: 9, a cDNA of SEQ ID NO: 10 or a homologous sequence; and
a secretion sequence disposed downstream of the transcription initiation sequence and upstream from the UBE3A sequence;
the amount of the vector being effective at expressing a secreted E6-associated protein (E6-AP) and rescuing E6-AP function in a subject with the UBE3A deficiency disease to improve motor function, associative learning or memory deficits in the subject.

2. The vector of claim 1, wherein the secretion sequence is GDNF, insulin or IgK.

3. The vector of claim 1, wherein the transcription initiation sequence is a cytomegalovirus chicken-beta actin hybrid promoter, or human ubiquitin c promoter.

4. The vector of claim 3, further comprising a cytomegalovirus immediate-early enhancer sequence disposed upstream of the transcription initiation sequence.

5. The vector of claim 4, further comprising a woodchuck hepatitis post-transcriptional regulatory element.

6. A method of improving motor function in a patient having a ubiquitin protein ligase E3A (UBE3A) deficiency disease comprising:
administering to the patient a therapeutically effective amount of a secreted UBE3A (SUB) adeno-associated viral (AAV) vector, the vector comprising:
a transcription initiation sequence;
a UBE3A sequence disposed downstream of the transcription initiation sequence, wherein the UBE3A sequence is a cDNA of SEQ ID NO: 1, SEQ ID NO: 2, SEQ ID NO: 7, a cDNA of SEQ ID NO: 8, SEQ ID NO: 9, a cDNA of SEQ ID NO: 10 or a homologous sequence; and
a secretion sequence disposed downstream of the transcription initiation sequence and upstream from the UBE3A sequence.
wherein the vector is administered via intracerebroventricular (ICV) injection, hippocampal injection, or a combination thereof to improve the motor function of the patient.

7. The method of claim 6, wherein the vector further comprises a cell uptake sequence disposed downstream of the transcription initiation sequence and between the secretion sequence and the UBE3A sequence to form a Secreted TAT UBE3A (STUB) vector.

8. The method of claim 7, wherein the cell uptake sequence is SEQ ID NO: 4 or a homologous sequence.

9. The method of claim 6, wherein the secretion sequence is SEQ ID NO: 3, SEQ ID NO: 5, SEQ ID NO: 6, or a homologous sequence.

10. The method of claim 6, wherein the transcription initiation sequence is a cytomegalovirus chicken-beta actin hybrid promoter, or human ubiquitin c promoter.

11. The method of claim 6, wherein the vector further comprises a cytomegalovirus immediate-early enhancer sequence disposed upstream of the transcription initiation sequence.

12. The method of claim 6, wherein the vector further comprises a woodchuck hepatitis post-transcriptional regulatory element.

13. The method of claim 6, wherein the UBE3A deficiency disease is selected from the group consisting of Angelman syndrome, Prader-Willi syndrome, and Huntington's disease.

14. A method of improving associative learning and memory deficits in a patient having a ubiquitin protein ligase E3A (UBE3A) deficiency disease comprising:
administering to the patient a therapeutically effective amount of an adeno-associated viral (AAV) vector containing a ubiquitin protein ligase E3A (UBE3A) gene;
wherein the vector is administered via intracerebroventricular (ICV) injection, hippocampal injection, or a combination thereof to increase the extracellular ubiquitin in the patient.

15. The method of claim 14, wherein the vector is a Secreted UBE3A (SUB) vector, the SUB vector comprising:
a transcription initiation sequence;
a UBE3A sequence disposed downstream of the transcription initiation sequence, wherein the UBE3A sequence is a cDNA of SEQ ID NO: 1, SEQ ID NO: 2, SEQ ID NO: 7, a cDNA of SEQ ID NO: 8, SEQ ID NO: 9, a cDNA of SEQ ID NO: 10 or a homologous sequence; and
a secretion sequence disposed downstream of the transcription initiation sequence and upstream from the UBE3A sequence.

16. The method of claim 15, wherein the vector further comprises a cell uptake sequence disposed downstream of the transcription initiation sequence and between the secretion sequence and the UBE3A sequence to form a Secreted TAT UBE3A (STUB) vector.

17. The method of claim 16, wherein the cell uptake sequence is SEQ ID NO: 4 or a homologous sequence.

18. The method of claim 15, wherein the secretion sequence is SEQ ID NO: 3, SEQ ID NO: 5, SEQ ID NO: 6, or a homologous sequence.

19. The method of claim 15, wherein the vector further comprises a cytomegalovirus immediate-early enhancer sequence disposed upstream of the transcription initiation sequence.

20. The method of claim 14, wherein the UBE3A deficiency disease is selected from the group consisting of Angelman syndrome, Prader-Willi syndrome, and Huntington's disease.
